(19) United States
(12) Patent Application Publication
(10) Pub. No.: US 2018/0065917 A1
(43) Pub. Date: Mar. 8, 2018

(54) IMMUNE CHECKPOINT INHIBITORS, COMPOSITIONS AND METHODS THEREOF

(71) Applicant: Polaris Pharmaceuticals, Inc., San Diego, CA (US)

(72) Inventors: Stephen E. WEBBER, San Diego, CA (US); Robert J. ALMASSY, Vista, CA (US)

(73) Assignee: Polaris Pharmaceuticals, Inc., Kitakyushu-shi (JP)

(21) Appl. No.: 15/691,891

(22) Filed: Aug. 31, 2017

Related U.S. Application Data
(60) Provisional application No. 62/382,842, filed on Sep. 2, 2016.

(51) Int. Cl. C07C 217/58; C07D 211/60; C07D 213/69; C07C 233/36; C07D 207/16; C07C 225/06; C07D 401/14; C07C 311/03

(52) U.S. Cl. CPC C07C 217/58; C07D 211/60; C07D 213/69; C07C 311/03; C07D 207/16; C07C 225/06; C07D 401/14; C07C 233/36

(57) ABSTRACT

The present invention provides synthesis, pharmaceutically acceptable formulations and uses of compounds in accordance with Formula (I), or a stereoisomer, a tautomer or a pharmaceutically acceptable salt thereof.

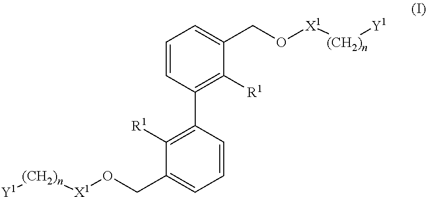

For Formula (I) compounds R¹, R², X¹, Y¹ and n are as defined in the specification. The inventive Formula (I) compounds are inhibitors of the PD-1/PD-L1 protein/protein binding or functional interaction and find utility in any number of therapeutic applications, including but not limited to treatment of proliferative disorders such as cancer and infectious diseases.

### CROSS-REFERENCE TO RELATED APPLICATION

The present application claims the benefit of U.S. Provisional Application No. 62/382,842, filed Sep. 2, 2016, which is herein incorporated by reference in its entirety.

### BACKGROUND

Programmed death-ligand 1 (PD-L1) also known as cluster of differentiation 274 (CD274) or B7 homolog 1 (B7-H1) is a 40 kDa type 1 transmembrane protein. PD-L1 is expressed on a variety of cell types, including placenta, vascular endothelium, pancreatic islet cells, muscle, hepatocytes, epithelium, and mesenchymal stem cells, as well as on B cells, T cells, dendritic cells, macrophages, and mast cells. Interaction of PD-L1 with programmed death receptor (PD-1; also known as CD279) expressed on T cells (particularly on activated T cells) results in reduced T-cell proliferation and cytokine expression to help control local inflammatory responses and maintain self-tolerance. Tumor cells have co-opted the PD-1/PD-L1 regulatory mechanism to avoid immunologic surveillance, facilitating cancer growth.

Blocking the PD-1/PD-L1 interaction would restore the function of chronically exhausted tumor-specific T cells and decrease tumor-induced immune suppression.

General examples of PD-L1 antagonists or inhibitors include an antibody or antigen-binding fragment or small molecule that specifically binds to PD-L1 and reduces one or more of its immune-suppressive activities, for example, its binding to the PD-1 receptor. Specific examples of PD-L1 antagonists include the antibodies atezolizumab (MPDL3280A), avelumab (MSB0010718C), and durvalumab (MEDI4736), and antigen-binding fragments thereof.

However, there remains a significant need for compounds that effectively and specifically inhibit the interaction between PD-1 and PD-L1 in a clinical setting, as well as for associated compositions and methods. The present invention satisfies this need and provides further related advantages.

### SUMMARY

The present invention generally relates to compounds useful as inhibitors of the functional interaction between PD-L1 and its receptors programmed cell death protein 1 (PD-1) and CD80 (B7-1). Such compounds may either completely disrupt binding of PD-L1 to PD-1 and/or CD80 or block or attenuate signal transduction through these receptors. Inhibitors of the interaction between PD-L1 and PD-1 are useful in the treatment of cancers and infectious diseases.

In one embodiment the invention is directed to compounds according to Formula (I):

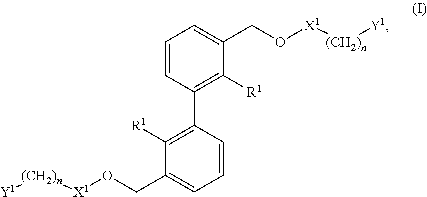

wherein
X¹ is aryl or heteroaryl,
Y¹ is NH(R²), N(R²)(R³) or heterocyclyl,
R¹ is H, CN, halogen, (C₁-C₈)alkyl or (C₁-C₈)haloalkyl,
R² and R³ are independently H, (C₁-C₈)alkyl, C(O)alkyl, C(O)CO₂H, or (SO₂)alkyl, or R² and R³ combine together with the nitrogen atom to form a heterocyclyl,
R⁴ is H, OH or (C₁-C₈)alkyl, and
n is an integer 0, 1 or 2,

wherein any alkyl, aryl, heterocyclyl or heteroaryl is optionally substituted with 1, 2 or 3 groups selected from OH, CN, NO₂, halogen, (C₁-C₃)alkyl, (C₁-C₃)haloalkyl, (C₃-C₆)cycloalkyl, alkenyl, alkynyl, O—(C₁-C₃)alkyl, O—(C₁-C₃)haloalkyl, O-(alkylene)aryl, O-(alkylene)heteroaryl, S—(C₁-C₈)alkyl, S—(C₁-C₈)haloalkyl, (SO₂)alkyl, C(O)OR⁴, C(O)NR⁴R⁴, C(O)NR⁴OR⁴, C(O)NR⁴(alkylene)OH, C(O)NR⁴(alkylene)N(R⁴)₂, C(O)NR⁴(SO₂)R⁴, NR⁴R⁴, NR⁴(CO)OR⁴ or NH(CO)R⁴, wherein the two R⁴'s of C(O)NR⁴R⁴, NR⁴R⁴ or C(O)NR⁴OR⁴ can optionally combine together with the nitrogen atom to form a heterocyclyl,

or a stereoisomer, a tautomer or a pharmaceutically acceptable salt thereof.

In one embodiment X¹ is aryl.

In another embodiment X¹ is aryl optionally substituted by OH, CN, halogen, (C₁-C₃)alkyl, (C₁-C₃)haloalkyl, alkenyl, alkynyl, O—(C₁-C₃)alkyl, O—(C₁-C₃)haloalkyl, S—(C₁-C₈)alkyl or S—(C₁-C₈)haloalkyl.

In another embodiment X¹ is aryl optionally substituted by halogen, (C₁-C₃)alkyl or (C₁-C₃)haloalkyl.

In one embodiment Y¹ is NH(R²) or N(R²)(R³).

In one embodiment R¹ is (C₁-C₈)alkyl or halogen.

In another embodiment R¹ is CH₃ or Cl.

In one embodiment R² and R³ are independently H or (C₁-C₈)alkyl or R² and R³ combine together with the nitrogen atom to form a heterocyclyl.

In another embodiment R² and R³ are independently H or (C₁-C₈)alkyl, wherein (C₁-C₈)alkyl is optionally substituted by OH or C(O)OH.

In another embodiment R² and R³ combine together with the nitrogen atom to form a heterocyclyl, optionally substituted by C(O)OH.

In one embodiment “n” is 1.

In one embodiment the compounds of the invention are
(2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(piperidine-2-carboxylic acid),
(2R,2′R)-1,1′-(((((2,2′-Dimethyl-[1,1′-biphenyl]-3,3′-diyl)bis(methylene))bis(oxy))bis(3-bromo-4,1-phenylene))bis(methylene))bis(piperidine-2-carboxylic acid),
N,N′-(((((((2,2′-Dimethyl-[1,1′-biphenyl]-3,3′-diyl)bis(methylene))bis(oxy))bis(2-methoxypyridine-6,3-diyl))bis(methylene))bis(azanediyl))bis(ethane-2, 1-diyl))diacetamide,
Dimethyl 1,1′-(((((2,2′-dimethyl-[1,1′-biphenyl]-3,3′-diyl)bis(methylene))bis(oxy))bis(3-bromo-4,1-phenylene))bis(methylene))(2S,2′S)-bis(piperidine-2-carboxylate),
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(ethan-1-ol)
N,N′-(((((((2,2′-Dimethyl-[1,1′-biphenyl]-3,3′-diyl)bis(methylene))bis(oxy))bis(3-bromo-4,1-phenylene))bis(methylene))bis(azanediyl))bis(ethane-2,1-diyl))diacetamide,
(2S,2′S,4S,4′S)-1,1′-(((((2,2′-Dimethyl-[1,1′-biphenyl]-3,3′-diyl)bis(methylene))bis(oxy))bis(3-bromo-4,1-phenylene))bis(methylene))bis(4-hydroxypyrrolidine-2-carboxylic acid),
(2S,2′S)-1,1′-(((([1,1′-Biphenyl]-3,3′-diylbis(methylene))bis(oxy))bis(3-bromo-4,1-phenylene))bis(methylene))bis(piperidine-2-carboxylic acid),
(2S,2′S)-1,1′-(((((2,2′-Dichloro-[1,1′-biphenyl]-3,3′-diyl)bis(methylene))bis(oxy))bis(3-bromo-4,1-phenylene))bis(methylene))bis(piperidine-2-carboxylic acid),
(2S,2′S)-1,1′-(((((2,2′-Difluoro-[1,1′-biphenyl]-3,3′-diyl)bis(methylene))bis(oxy))bis(3-bromo-4,1-phenylene))bis(methylene))bis(piperidine-2-carboxylic acid),
(2S,2′S)-1,1′-(((((2,2′-Dimethyl-[1,1′-biphenyl]-3,3′-diyl)bis(methylene))bis(oxy))bis(3-methyl-4,1-phenylene))bis(methylene))bis(piperidine-2-carboxylic acid),
(2S,2′S)-1,1′-(((((2,2′-Dimethyl-[1,1′-biphenyl]-3,3′-diyl)bis(methylene))bis(oxy))bis(3-chloro-4,1-phenylene))bis(methylene))bis(piperidine-2-carboxylic acid),
(2S,2′S)-1,1′-(((((2,2′-Dimethyl-[1,1′-biphenyl]-3,3′-diyl)bis(methylene))bis(oxy))bis(3-(trifluoromethyl)-4,1-phenylene))bis(methylene))bis(piperidine-2-carboxylic acid),
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-methylpropanoic acid) di-trifluoroacetic acid salt,
(2S,2′S)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))dipropionic acid di-trifluoroacetic acid salt,
(2R,2′R)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))dipropionic acid ditrifluoroacetic acid salt,
(2R,2′R)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-hydroxypropanoic acid) ditrifluoroacetic acid salt,
(2R,2′R,3S,3′S)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-hydroxybutanoic acid) di-trifluoroacetic acid salt,
(2S,2′S)-1,1′-(((((2,2′-Dimethyl-[1,1′-biphenyl]-3,3′-diyl)bis(methylene))bis(oxy))bis(3-fluoro-4,1-phenylene))bis(methylene))bis(piperidine-2-carboxylic acid),
(2S,2′S)-1,1′-(((((2,2′-Dimethyl-[1,1′-biphenyl]-3,3′-diyl)bis(methylene))bis(oxy))bis(3-methoxy-4,1-phenylene))bis(methylene))bis(piperidine-2-carboxylic acid),
(2S,2′S)-1,1′-(((((2,2′-Dimethyl-[1,1′-biphenyl]-3,3′-diyl)bis(methylene))bis(oxy))bis(3-hydroxy-4,1-phenylene))bis(methylene))bis(piperidine-2-carboxylic acid),
2,2′-((((((2,2′-Dimethyl-[1,1′-biphenyl]-3,3′-diyl)bis(methylene))bis(oxy))bis(2,6-dimethoxy-4,1-phenylene))bis(methylene))bis(azanediyl))bis(ethan-1-ol),
(2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(piperidine-2-carboxamide),
(2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(N-(2-hydroxyethyl)piperidine-2-carboxamide),
(2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(N-(2-(dimethylamino)ethyl)piperidine-2-carboxamide),
(2S,2′S)-1,1′-(((((2,2′-Dimethyl-[1,1′-biphenyl]-3,3′-diyl)bis(methylene))bis(oxy))bis(3-ethynyl-4,1-phenylene))bis(methylene))bis(piperidine-2-carboxylic acid) di-trifluoroacetic acid,
(2S,2′S)-1,1′-(((((2,2′-Dimethyl-[1,1′-biphenyl]-3,3′-diyl)bis(methylene))bis(oxy))bis(3-ethyl-4,1-phenylene))bis(methylene))bis(piperidine-2-carboxylic acid) di-trifluoroacetic acid,
(2S,2′S)-1,1′-(((((2,2′-Dimethyl-[1,1′-biphenyl]-3,3′-diyl)bis(methylene))bis(oxy))bis(3-(methylthio)-4,1-phenylene))bis(methylene))bis(piperidine-2-carboxylic acid) di-trifluoroacetic acid,
(2S,2′S)-1,1′-(((((2,2′-Dimethyl-[1,1′-biphenyl]-3,3′-diyl)bis(methylene))bis(oxy))bis(3-(difluoromethyl)-4,1-phenylene))bis(methylene))bis(piperidine-2-carboxylic acid) di-trifluoroacetic acid salt,
2,2′,2″,2′″-((((((2,2′-Dimethyl-[1,1′-biphenyl]-3,3′-diyl)bis(methylene))bis(oxy))bis(3-bromo-4,1-phenylene))bis(methylene))bis(azanetriyl))tetraacetic acid di-trifluoroacetic acid salt,
2,2′,2″,2′″-((((((2,2′-Dimethyl-[1,1′-biphenyl]-3,3′-diyl)bis(methylene))bis(oxy))bis(3-bromo-4,1-phenylene))bis(methylene))bis(azanetriyl))tetrakis(ethan-1-ol) di-trifluoroacetic acid salt,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(propane-1,3-diol) di-trifluoroacetic acid salt,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-methylpropane-1,3-diol) di-trifluoroacetic acid salt,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))dimalonic acid,
((((2,2′-Dimethyl-[1,1′-biphenyl]-3,3′-diyl)bis(methylene))bis(oxy))bis(3-bromo-4,1-phenylene))dimethanamine,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-oxoacetic acid),
3,3′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-oxopropanoic acid),
N,N′-(((((2,2′-Dimethyl-[1,1′-biphenyl]-3,3′-diyl)bis(methylene))bis(oxy))bis(3-bromo-4,1-phenylene))bis(methylene))dimethanesulfonamide,
2,2′-(((3-Bromo-4-((3′-((2-bromo-4-((hydrosulfonylamino)methyl)phenoxy)methyl)-2,2′-dimethyl-[1,1′-biphenyl]-3-yl)methoxy)benzyl)amino)sulfonyl)diacetic acid,
(2S,2′S)-1,1′-(((((2,2′-Bis(trifluoromethyl)-[1,1′-biphenyl]-3,3′-diyl)bis(methylene))bis(oxy))bis(3-bromo-4,1-phenylene))bis(methylene))bis(piperidine-2-carboxylic acid),
(2S,2′S)-1,1′-(((((2,2′-Dicyano-[1,1′-biphenyl]-3,3′-diyl)bis(methylene))bis(oxy))bis(3-bromo-4,1-phenylene))bis(methylene))bis(piperidine-2-carboxylic acid),
(2S,2′S)-1,1′-(((((2,2′-dimethyl-[1,1′-biphenyl]-3,3′-diyl)bis(methylene))bis(oxy))bis(3-cyano-4,1-phenylene))bis(methylene))bis(piperidine-2-carboxylic acid),
(2S,2′S)-1,1′-(((((2,2′-Dimethyl-[1,1′-biphenyl]-3,3′-diyl)bis(methylene))bis(oxy))bis(2-((5-cyanopyridin-3-yl)methoxy)-5-fluoro-4, 1-phenylene))bis(methylene))bis(piperidine-2-carboxylic acid),
(2S,2′S)-1,1′-(((((2,2′-Dimethyl-[1,1′-biphenyl]-3,3′-diyl)bis(methylene))bis(oxy))bis(5-chloro-2-((5-cyanopyridin-3-yl)methoxy)-4, 1-phenylene))bis(methylene))bis(piperidine-2-carboxylic acid),
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-methylmalonic acid),
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-hydroxy-2-(hydroxymethyl)propanoic acid),
1,1′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(cyclopropane-1-carboxylic acid) di-trifluoroacetic acid,
1,1′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(cyclobutane-1-carboxylic acid) di-trifluoroacetic acid,
1,1′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(cyclopentane-1-carboxylic acid) di-trifluoroacetic acid,
(2S,2′S)-1,1′-(((((2,2′-Dichloro-[1,1′-biphenyl]-3,3′-diyl)bis(methylene))bis(oxy))bis(3-(trifluoromethyl)-4,1-phenylene))bis(methylene))bis(piperidine-2-carboxylic acid),
2,2′-((((((2,2′-Dichloro-[1,1′-biphenyl]-3,3′-diyl)bis(methylene))bis(oxy))bis(3-(trifluoromethyl)-4,1-phenylene))bis(methylene))bis(azanediyl))bis(2-methylpropanoic acid) di-trifluoroacetic acid salt,
(2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(N-(2-(dimethylamino)ethyl)-N-methylpiperidine-2-carboxamide),
(2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(N-hydroxypiperidine-2-carboxamide),
1,1′-((2S,2′S)-(((((2,2′-Dimethyl-[1,1′-biphenyl]-3,3′-diyl)bis(methylene))bis(oxy))bis(3-bromo-4,1-phenylene))bis(methylene))bis(piperidine-1,2-diyl))bis(N-(methylsulfonyl)formamide),
(2S,2′S)-1,1′-(((((2,2′-dimethyl-[1,1′-biphenyl]-3,3′-diyl)bis(methylene))bis(oxy))bis(3-cyano-4,1-phenylene))bis(methylene))bis(piperidine-2-carboxylic acid) di-trifluoroacetic acid salt,
(2S,2′S)-1,1′-(((((2,2′-Dimethyl-[1,1′-biphenyl]-3,3′-diyl)bis(methylene))bis(oxy))bis(5-chloro-2-((5-cyanopyridin-3-yl)methoxy)-4, 1-phenylene))bis(methylene))bis(piperidine-2-carboxylic acid) di-trifluoroacetic acid salt,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))dimalonic acid,
((((2,2′-Dimethyl-[1,1′-biphenyl]-3,3′-diyl)bis(methylene))bis(oxy))bis(3-bromo-4,1-phenylene))dimethanamine,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-oxoacetic acid),
3,3′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-oxopropanoic acid),
N,N′-(((((2,2′-Dimethyl-[1,1′-biphenyl]-3,3′-diyl)bis(methylene))bis(oxy))bis(3-bromo-4,1-phenylene))bis(methylene))dimethanesulfonamide,
2,2′-(((3-Bromo-4-((3′-((2-bromo-4-((hydrosulfonylamino)methyl)phenoxy)methyl)-2,2′-dimethyl-[1,1′-biphenyl]-3-yl)methoxy)benzyl)amino)sulfonyl)diacetic acid,
(2S,2′S)-1,1′-(((((2,2′-Bis(trifluoromethyl)-[1,1′-biphenyl]-3,3′-diyl)bis(methylene))bis(oxy))bis(3-bromo-4,1-phenylene))bis(methylene))bis(piperidine-2-carboxylic acid),
(2S,2′S)-1,1′-(((((2,2′-Dicyano-[1,1′-biphenyl]-3,3′-diyl)bis(methylene))bis(oxy))bis(3-bromo-4,1-phenylene))bis(methylene))bis(piperidine-2-carboxylic acid),
(2S,2′S)-1,1′-(((((2,2′-Dimethyl-[1,1′-biphenyl]-3,3′-diyl)bis(methylene))bis(oxy))bis(2-((5-cyanopyridin-3-yl)methoxy)-5-fluoro-4, 1-phenylene))bis(methylene))bis(piperidine-2-carboxylic acid) di-trifluoroacetic acid salt,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-methylmalonic acid),
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-hydroxy-2-(hydroxymethyl)propanoic acid),
1,1′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(cyclopropane-1-carboxylic acid) di-trifluoroacetic acid,
1,1′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(cyclobutane-1-carboxylic acid) di-trifluoroacetic acid,
1,1′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(cyclopentane-1-carboxylic acid) di-trifluoroacetic acid,
(2S,2′S)-1,1′-(((((2,2′-Dichloro-[1,1′-biphenyl]-3,3′-diyl)bis(methylene))bis(oxy))bis(3-(trifluoromethyl)-4, 1-phenylene))bis(methylene))bis(piperidine-2-carboxylic acid),
2,2′-((((((2,2′-Dichloro-[1,1′-biphenyl]-3,3′-diyl)bis(methylene))bis(oxy))bis(3-(trifluoromethyl)-4,1-phenylene))bis(methylene))bis(azanediyl))bis(2-methylpropanoic acid) di-trifluoroacetic acid salt,
(2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(N-(2-(dimethylamino)ethyl)-N-methylpiperidine-2-carboxamide),
(2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(N-hydroxypiperidine-2-carboxamide),
(2S,2′S)-1,1′-(((((2,2′-dimethyl-[1,1′-biphenyl]-3,3′-diyl)bis(methylene))bis(oxy))bis(3-bromo-4,1-phenylene))bis(methylene))bis(N-methoxypiperidine-2-carboxamide),
1,1′-((2S,2′S)-(((((2,2′-Dimethyl-[1,1′-biphenyl]-3,3′-diyl)bis(methylene))bis(oxy))bis(3-bromo-4,1-phenylene))bis(methylene))bis(piperidine-1,2-diyl))bis(N-(methylsulfonyl)formamide),
2,2′-((((((2,2′-dimethyl-[1,1′-biphenyl]-3,3′-diyl)bis(methylene))bis(oxy))bis(3-(difluoromethyl)-4,1-phenylene))bis(methylene))bis(azanediyl))bis(2-methylpropane-1,3-diol) di-trifluoroacetic acid salt,
2,2′-((((((2,2′-dimethyl-[1,1′-biphenyl]-3,3′-diyl)bis(methylene))bis(oxy))bis(3-(difluoromethyl)-4,1-phenylene))bis(methylene))bis(methylazanediyl))bis(2-methylpropane-1,3-diol) di-trifluoroacetic acid salt,
2,2′-((((((2,2′-dichloro-[1,1′-biphenyl]-3,3′-diyl)bis(methylene))bis(oxy))bis(3-bromo-4,1-phenylene))bis(methylene))bis(azanediyl))bis(2-methylpropane-1,3-diol) di-trifluoroacetic acid salt,
2,2′-((((((2,2′-dichloro-[1,1′-biphenyl]-3,3′-diyl)bis(methylene))bis(oxy))bis(3-(difluoromethyl)-4,1-phenylene))bis(methylene))bis(azanediyl))bis(2-methylpropane-1,3-diol) di-trifluoroacetic acid salt,
2,2′-((((((2,2′-dimethyl-[1,1′-biphenyl]-3,3′-diyl)bis(methylene))bis(oxy))bis(3-(difluoromethyl)-4,1-phenylene))bis(methylene))bis(azanediyl))bis(2-(hydroxymethyl)propane-1,3-diol) di-trifluoroacetic acid salt,
(2S,2′S)-1,1′-(((((2,2′-dichloro-[1,1′-biphenyl]-3,3′-diyl)bis(methylene))bis(oxy))bis(3-(difluoromethyl)-4,1-phenylene))bis(methylene))bis(piperidine-2-carboxylic acid), or
(2S,2′S)-1,1′-(((((2,2′-dichloro-[1,1′-biphenyl]-3,3′-diyl)bis(methylene))bis(oxy))bis(2-((5-cyanopyridin-3-yl)methoxy)-5-(difluoromethyl)-4, 1-phenylene))bis(methylene))bis(piperidine-2-carboxylic acid) di-trifluoroacetic acid salt,
   or a stereoisomer, a tautomer or a pharmaceutically acceptable salt thereof.

The present invention also provides a pharmaceutical composition comprising (i) a therapeutically effective amount of at least one compound according to Formula (I) or a stereoisomer, a tautomer or a pharmaceutically acceptable salt thereof; (ii) in combination with a pharmaceutically acceptable carrier, diluent or excipient.

According to yet another embodiment the invention provides a method for treating a PD-1/PD-LI dependent condition in a mammal in need thereof comprising administering to the mammal (i) a therapeutically effective amount of at least one compound according to Formula (I) or a stereoisomer, tautomer or pharmaceutically acceptable salt thereof, or (ii) a pharmaceutical composition in accordance with the invention.

The above embodiments and other aspects of the invention are readily apparent in the detailed description that follows. To this end, various references are set forth herein which describe in more detail certain background information, procedures, compounds and/or compositions, and are each hereby incorporated by reference in their entireties.

### DETAILED DESCRIPTION

In the following description certain specific details are set forth in order to provide a thorough understanding of various embodiments of the invention. However, one skilled in the art will understand that the invention may be practiced without these details. Unless the context requires otherwise, throughout the present specification and claims, the word “comprise” and variations thereof, such as, “comprises” and “comprising” are to be construed in an open, inclusive sense (i.e., as “including, but not limited to”).

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

### Definitions

As used herein, and unless noted to the contrary, the following terms and phrases have the meaning noted below.

“Amino” refers to the —NH₂ substituent.

“Aminocarbonyl” refers to the —C(O)NH₂ substituent.

“Carboxyl” refers to the —CO₂H substituent.

“Carbonyl” refers to a —C(O)— or —C(═O)— group. Both notations are used interchangeably within the specification.

“Cyano” refers to the —C≡N substituent.

“Acetyl” refers to the —C(O)CH₃ substituent.

“Hydroxy” or “hydroxyl” refers to the —OH substituent.

“Oxo” refers to a ═O substituent.

“Thio” or “thiol” refer to a —SH substituent.

“Alkyl” refers to a saturated, straight or branched hydrocarbon chain radical consisting solely of carbon and hydrogen atoms, having from one to twelve carbon atoms (C₁-C₁₂ alkyl), from one to eight carbon atoms (C₁-C₈ alkyl) or from one to six carbon atoms (C₁-C₆ alkyl), and which is attached to the rest of the molecule by a single bond. Exemplary alkyl groups include methyl, ethyl, n-propyl, 1-methylethyl (iso-propyl), n-butyl, n-pentyl, 1,1-dimethylethyl (t-butyl), 3-methylhexyl, 2-methylhexyl, and the like. Moieties with which the alkyl group can be substituted with are selected from but not necessarily limited to the group consisting of hydroxyl, amino, alkylamino, arylamino, alkoxy, thioalkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., “Protective Groups in Organic Synthesis”, John Wiley and Sons, Second Edition, 1991.

“Lower alkyl” has the same meaning as alkyl defined above but having from one to three carbon atoms (C₁-C₃ alkyl).

“Alkenyl” refers to an unsaturated alkyl group having at least one double bond and from two to twelve carbon atoms (C₂-C₁₂ alkenyl), from two to eight carbon atoms (C₂-C₈ alkenyl) or from two to six carbon atoms (C₂-C₆ alkenyl), and which is attached to the rest of the molecule by a single bond, e.g., ethenyl, propenyl, butenyl, pentenyl, hexenyl, and the like.

“Alkynyl” refers to an unsaturated alkyl group having at least one triple bond and from two to twelve carbon atoms (C₂-C₁₂ alkynyl), from two to ten carbon atoms (C₂-C₁₀ alkynyl) from two to eight carbon atoms (C₂-C₈ alkynyl) or from two to six carbon atoms (C₂-C₆ alkynyl), and which is attached to the rest of the molecule by a single bond, e.g., ethynyl, propynyl, butynyl, pentynyl, hexynyl, and the like.

“Alkylene” or “alkylene chain” refers to a straight or branched divalent hydrocarbon (alkyl) chain linking the rest of the molecule to a radical group, consisting solely of carbon and hydrogen, respectively. Alkylenes can have from one to twelve carbon atoms, e.g., methylene, ethylene, propylene, n-butylene, and the like. The alkylene chain is attached to the rest of the molecule through a single or double bond. The points of attachment of the alkylene chain to the rest of the molecule can be through one carbon or any two carbons within the chain. “Optionally substituted alkylene” refers to alkylene or substituted alkylene.

“Alkoxy” refers to a radical of the formula —ORₐ where Rₐ is an alkyl having the indicated number of carbon atoms as defined above. Examples of alkoxy groups include without limitation —O-methyl (methoxy), —O-ethyl (ethoxy), —O-propyl (propoxy), —O-isopropyl (iso propoxy) and the like.

“Aryl” refers to a hydrocarbon ring system radical comprising hydrogen, 6 to 18 carbon atoms and at least one aromatic ring. Exemplary aryls are hydrocarbon ring system radical comprising hydrogen and 6 to 9 carbon atoms and at least one aromatic ring; hydrocarbon ring system radical comprising hydrogen and 9 to 12 carbon atoms and at least one aromatic ring; hydrocarbon ring system radical comprising hydrogen and 12 to 15 carbon atoms and at least one aromatic ring; or hydrocarbon ring system radical comprising hydrogen and 15 to 18 carbon atoms and at least one aromatic ring. For purposes of this invention, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. “Optionally substituted aryl” refers to an aryl group or a substituted aryl group. The aryl group can be substituted with, but not necessarily limited to, one or more moieties selected from the group consisting of hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., “Protective Groups in Organic Synthesis”, John Wiley and Sons, Second Edition, 1991.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo (bromine), chloro (chlorine), fluoro (fluorine), or iodo (iodine).

“Haloalkyl” refers to an alkyl radical having the indicated number of carbon atoms, as defined herein, wherein one or more hydrogen atoms of the alkyl group are substituted with a halogen (halo radicals), as defined above. The halogen atoms can be the same or different. Exemplary haloalkyls are trifluoromethyl, difluoromethyl, trichloromethyl, 2,2,2-trifluoroethyl, 1,2-difluoroethyl, 3-bromo-2-fluoropropyl, 1,2-dibromoethyl, and the like.

“Heterocyclyl”, heterocycle”, or “heterocyclic ring” refers to a stable 3- to 18-membered saturated or unsaturated radical which consists of two to twelve carbon atoms and from one to six heteroatoms, for example, one to five heteroatoms, one to four heteroatoms, one to three heteroatoms, or one to two heteroatoms selected from the group consisting of nitrogen, oxygen and sulfur. Exemplary heterocycles include without limitation stable 3-15 membered saturated or unsaturated radicals, stable 3-12 membered saturated or unsaturated radicals, stable 3-9 membered saturated or unsaturated radicals, stable 8-membered saturated or unsaturated radicals, stable 7-membered saturated or unsaturated radicals, stable 6-membered saturated or unsaturated radicals, or stable 5-membered saturated or unsaturated radicals.

Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of non-aromatic heterocyclyl radicals include, but are not limited to, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, thietanyl, trithianyl, tetrahydropyranyl, thiomorpholinyl, thiamorpholinyl, 1-oxo-thiomorpholinyl, and 1,1-dioxo-thiomorpholinyl. Heterocyclyls include heteroaryls as defined herein, and examples of aromatic heterocyclyls are listed in the definition of heteroaryls below.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical may be a stable 5-12 membered ring, a stable 5-10 membered ring, a stable 5-9 membered ring, a stable 5-8 membered ring, a stable 5-7 membered ring, or a stable 6 membered ring that comprises at least 1 heteroatom, at least 2 heteroatoms, at least 3 heteroatoms, at least 4 heteroatoms, at least 5 heteroatoms or at least 6 heteroatoms. Heteroaryls may be a monocyclic, bicyclic, tricyclic or tetracyclic ring system, which may include fused or bridged ring systems; and the nitrogen, 2 carbon or sulfur atoms in the heteroaryl radical may be optionally oxidized; the nitrogen atom may be optionally quaternized. The heteroatom may be a member of an aromatic or non-aromatic ring, provided at least one ring in the heteroaryl is aromatic. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl).

The term “compound” refers to a chemical entity, whether in the solid, liquid or gaseous phase, and whether in a crude mixture or purified and isolated. The compound of the invention can exist in various isomeric forms, as well as in one or more tautomeric forms, including both single tautomers and mixtures of tautomers. The term “isomer” is intended to encompass all isomeric forms of a compound of this invention, including tautomeric forms of the compound.

Some compounds described here can have asymmetric centers and therefore exist in different enantiomeric and diastereomeric forms. A compound of the invention can be in the form of an optical isomer or a diastereomer. Accordingly, the invention encompasses compounds of the invention and their uses as described herein in the form of their optical isomers, diastereoisomers and mixtures thereof, including a racemic mixture. Optical isomers of the compounds of the invention can be obtained by known techniques such as asymmetric synthesis, chiral chromatography, or via chemical separation of stereoisomers through the employment of optically active resolving agents.

Unless otherwise indicated, “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

In this description, a “pharmaceutically acceptable salt” is a pharmaceutically acceptable, organic or inorganic acid or base salt of a compound of the invention. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, trifluoroacetate and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In certain embodiments, such terms refer to minimizing the spread or worsening of the disease resulting from the administration of one or more prophylactic or therapeutic agents to a patient with such a disease. In the context of the present invention the terms “treat”, “treating” and “treatment” also refer to:
(i) preventing the disease or condition from occurring in a mammal, in particular, when such mammal is predisposed to the condition but has not yet been diagnosed as having it;
(ii) inhibiting the disease or condition, i.e., arresting its development;
(iii) relieving the disease or condition, i.e., causing regression of the disease or condition; or
(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The terms “modulate”, “modulation” and the like refer to the ability of a compound to increase or decrease the function, or activity of, for example, the interaction between PD-1 and PD-L. “Modulation”, in its various forms, is intended to encompass inhibition, antagonism, partial antagonism, activation, agonism and/or partial agonism of the activity associated with PD-1 and PD-L1. PD-1 and PD-L1 inhibitors are compounds that bind to, partially or totally block stimulation, decrease, prevent, delay activation, inactivate, desensitize, or down regulate signal transduction. The ability of a compound to modulate PD-1 or PD-L1 activity can be demonstrated in a suitable enzymatic assay or a suitable cell-based assay.

A “patient” or “subject” includes an animal, such as a human, cow, horse, sheep, lamb, pig, chicken, turkey, quail, cat, dog, mouse, rat, rabbit or guinea pig. The animal can be a mammal such as a non-primate and a primate (e.g., monkey and human). In one embodiment, a patient is a human, such as a human infant, child, adolescent or adult.

The term “prodrug” refers to a precursor of a drug, a compound which upon administration to a patient must undergo chemical conversion by metabolic processes before becoming an active pharmacological agent. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. Exemplary prodrugs of compounds in accordance with Formula (I) are esters, acetamides, and amides.

The inventive compounds according to Formula (I) may be isotopically-labeled by having one or more atoms replaced by an atom having a different atomic mass or mass number. Examples of isotopes that can be incorporated into compounds of according to Formula (I) include isotopes of hydrogen, carbon, nitrogen, oxygen, phosphorous, fluorine, chlorine, or iodine. Illustrative of such isotopes are ²H, ³H, ¹¹C, ¹³C, ¹⁴C, ¹³N, ¹⁵N, ¹⁵O, ¹⁷O, ¹⁸O, ³¹p, ³²P, ³⁵S, ¹⁸F, ³⁶Cl, ¹²³I, and ¹²⁵I, respectively. These radiolabeled compounds can be used to measure the biodistribution, tissue concentration and the kinetics of transport and excretion from biological tissues including a subject to which such a labeled compound is administered. Labeled compounds are also used to determine therapeutic effectiveness, the site or mode of action, and the binding affinity of a candidate therapeutic to a pharmacologically important target. Certain radioactive-labeled compounds according to Formula (I), therefore, are useful in drug and/or tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, affords certain therapeutic advantages resulting from the greater metabolic stability, for example, increased in vivo half-life of compounds containing deuterium. Substitution of hydrogen with deuterium may reduce dose required for therapeutic effect, and hence may be preferred in a discovery or clinical setting. Substitution with positron emitting isotopes, such as ¹C, ¹⁸F, ⁵O and ¹³N, provides labeled analogs of the inventive compounds that are useful in Positron Emission Tomography (PET) studies, e.g., for examining substrate receptor occupancy. Isotopically-labeled compounds according to Formula (I) can generally be prepared by conventional techniques known to those skilled in the art or by processes analogous to those described in the Preparations and Examples section as set out below using an appropriate isotopic-labeling reagent.

Embodiments of the invention disclosed herein are also meant to encompass the in vivo metabolic products of compounds according to Formula (I). Such products may result from, for example, the oxidation, reduction, hydrolysis, amidation, esterification, and like processes primarily due to enzymatic activity upon administration of a compound of the invention. Accordingly, the invention includes compounds that are produced as by-products of enzymatic or non-enzymatic activity on an inventive compound following the administration of such a compound to a mammal for a period of time sufficient to yield a metabolic product. Metabolic products, particularly pharmaceutically active metabolites are typically identified by administering a radiolabeled compound of the invention in a detectable dose to a subject, such as rat, mouse, guinea pig, monkey, or human, for a sufficient period of time during which metabolism occurs, and isolating the metabolic products from urine, blood or other biological samples that are obtained from the subject receiving the radiolabeled compound.

The invention also provides pharmaceutically acceptable salt forms of Formula (I) compounds. Encompassed within the scope of the invention are both acid and base addition salts that are formed by contacting a pharmaceutically suitable acid or a pharmaceutically suitable base with a compound of the invention.

To this end, a “pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, laurylsulfate, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, sterylsulfate, succinic acid, tartaric acid, thiocyanic acid, p-toluenesulfonic acid, trifluoroacetic acid, undecylenic acid, and the like.

Similarly, a “pharmaceutically acceptable base addition salt” refers to those salts which retain the biological effectiveness and properties of the free acids, which are not biologically or otherwise undesirable. These salts are prepared by addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, meglumine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the invention. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

Compounds of the invention or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

The term “tautomer” refers to a proton shift from one atom of a molecule to another atom of the same molecule.

The inventive compounds are synthesized using conventional synthetic methods, and more specifically using the general methods noted below.

### Pharmaceutical Formulations

In one embodiment, a compounds according to Formula (I) are formulated as pharmaceutically acceptable compositions that contain a Formula (I) compound in an amount effective to treat a particular disease or condition of interest upon administration of the pharmaceutical composition to a mammal. Pharmaceutical compositions in accordance with the present invention can comprise a Formula (I) compound in combination with a pharmaceutically acceptable carrier, diluent or excipient.

In this regard, a “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

Further, a “mammal” includes humans and both domestic animals such as laboratory animals and household pets (e.g., cats, dogs, swine, cattle, sheep, goats, horses, rabbits), and non-domestic animals such as wildlife and the like.

The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings of this invention.

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

As a solid composition for oral administration the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The pharmaceutical composition of the invention may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the invention may be prepared by any methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

In certain embodiments a pharmaceutical composition comprising a compound of Formula (I) is administered to a mammal in an amount sufficient to inhibit the PD-1/PD-L1 interaction upon administration, and preferably with acceptable toxicity to the same. The inhibition of PD-1 or PD-L1 interaction by Formula (I) compounds can be determined by one skilled in the art, for example, as described in the Examples below. Appropriate concentrations and dosages can be readily determined by one skilled in the art.

### Therapeutic Use

The compounds of the invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

“Effective amount” or “therapeutically effective amount” refers to that amount of a compound of the invention which, when administered to a mammal, preferably a human, is sufficient to effect treatment, as defined below, of a PD-1/PD-L1 associated condition or disease in the mammal, preferably a human. The amount of a compound of the invention which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

Compounds of the invention or pharmaceutically acceptable salt thereof may also be administered simultaneously with, prior to, or after administration of one or more other therapeutic agents. Such combination therapy includes administration of a single pharmaceutical dosage formulation which contains a compound of the invention and one or more additional active agents, as well as administration of the compound of the invention and each active agent in its own separate pharmaceutical dosage formulation. For example, a compound of the invention and the other active agent can be administered to the patient together in a single oral dosage composition such as a tablet or capsule, or each agent administered in separate oral dosage formulations. Where separate dosage formulations are used, the compounds of the invention and one or more additional active agents can be administered at essentially the same time, i.e., concurrently, or at separately staggered times, i.e., sequentially; combination therapy is understood to include all these regimens.

The compounds of Formula I inhibit the PD-1/PD-L1 protein/protein binding or functional interaction thereby preventing or reversing functional exhaustion of effector T cells. By restoring effector T cell function the compounds boost immune response against cancerous cells or infectious agents.

The compounds of Formula I are useful in treating, ameliorating, or reducing the symptoms or progression of diseases of uncontrolled cell growth, proliferation and/or survival, such as, for example, hematological tumors, solid tumors, circulating tumors and/or metastases thereof, including myeloproliferative disorders, leukemias and myelodysplastic syndrome, malignant lymphomas, for example, acute myelogenous (granulocytic) leukemia (AML), chronic myelogenous leukemia (CML), acute lymphocytic (lymphoblastic) leukemia (ALL), chronic lymphocytic leukemia (CLL), chronic idiopathic myelofibrosis, polycythemia vera, essential thrombocythemia, myeloid metaplasia, acute erythroblastic leukemia, Hodgkin's lymphoma, non-Hodgkin's lymphoma, hairy cell lymphoma, Burkitt's lymphoma, B-cell lymphoma, acute T-cell leukemia and T-cell lymphoma, head and neck tumors including brain tumors and brain metastases, tumors of the thorax including non-small cell and small cell lung tumors, gastrointestinal tumors, endocrine tumors, mammary and other gynecological tumors, urological tumors including renal, bladder and prostate tumors, skin tumors, and sarcomas, and/or metastases thereof.

Inhibitors of the PD-1/PD-L1 axis are suitable candidate therapeutics for treating cell proliferative disorders such as cancer. A wide variety of cancers, including solid tumors, lymphomas and leukemias, are amenable to the compositions and methods disclosed herein. Types of cancer that may be treated include, but are not limited to: adenocarcinoma of the breast, prostate, and colon; all forms of bronchogenic carcinoma of the lung; esophageal, myeloid; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, merkel cell, mucinous, non-small cell lung, oat cell, papillary, scirrhous, bronchiolar, bronchogenic, squamous cell, and transitional cell). Additional types of cancers that may be treated include: histiocytic disorders; leukemia; histiocytosis malignant; Hodgkin's disease; immunoproliferative small; non-Hodgkin's lymphoma; T-cell lymphoma, B-cell lymphoma, hairy cell lymphoma, Burkitt's lymphoma, plasmacytoma; reticuloendotheliosis; melanoma; chondroblastoma; chondroma; chondrosarcoma; fibroma; fibrosarcoma; giant cell tumors; histiocytoma; lipoma; liposarcoma; mesothelioma; myxoma; myxosarcoma; osteoma; osteosarcoma; chordoma; craniopharyngioma; dysgerminoma; hamartoma; mesenchymoma; mesonephroma; myosarcoma; ameloblastoma; cementoma; odontoma; teratoma; thymoma; trophoblastic tumor.

Other cancers that can be treated using the inventive compounds include without limitation adenoma; cholangioma; cholesteatoma; cyclindroma; cystadenocarcinoma; cystadenoma; granulosa cell tumor; gynandroblastoma; hepatoma; hidradenoma; islet cell tumor; Leydig cell tumor; papilloma; sertoli cell tumor; theca cell tumor; leimyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin.

In one embodiment the inventive compounds are candidate therapeutic agents for the treatment of cancers such as angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia.

In a particular embodiment the present disclosure provides methods for treating colon cancer, gastric cancer, thyroid cancer, lung cancer, leukemia, pancreatic cancer, melanoma, multiple melanoma, brain cancer, CNS cancer, renal cancer, prostate cancer, ovarian cancer, or breast cancer. Illustrative of the category “brain cancer” are glioblastomas, astrocytomas, medulloblastoma, meningiomas and other disease conditions related to brain cancer metastases. According to such a method, a therapeutically effective amount of at least one compound according to Formula (I) or a stereoisomer, tautomer or pharmaceutically acceptable salt thereof can be administered to a subject who has been diagnosed with a cell proliferative disease, such as a cancer. Alternatively, a pharmaceutical composition comprising at least one compound according to Formula I or a stereoisomer, tautomer or pharmaceutically acceptable salt thereof can be administered to a subject who has been diagnosed with cancer.

In certain embodiments the compounds in accordance with the invention are administered to a subject with cancer in conjunction with other conventional cancer therapies such as radiation treatment or surgery. Radiation therapy is well-known in the art and includes X-ray therapies, such as gamma-irradiation, and radiopharmaceutical therapies.

In certain embodiments the inventive Formula I compounds are used with at least one anti-cancer agent. Anti-cancer agents include chemotherapeutic drugs. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor.

Non-limiting examples of infectious viruses include adenovirus, bunyavirus (e.g., hantavirus), herpesvirus, papovavirus, paramyxovirus, picornavirus, rhabdovirus (e.g., rabies), orthomyxovirus (e.g., influenza), poxvirus (e.g., Vaccinia), reovirus, retrovirus, lentivirus (e.g., HIV), flavivirus (e.g., HCV), or the like).

The invention also in part pertains to methods for stimulating an immune response in a subject. Thus, provided are methods for determining whether a compound herein modulates an immune response, which comprise contacting a system with a compound described herein in an amount effective for restoring effector T cell function that have been repressed by PD-1/PD-L1 interaction. Signals effector T cell functional activity include, e.g., stimulation of T-cell proliferation, induction of cytokines, including, e.g., interleukin 2 (IL-2), interferon-γ and TNFα. Methods of assessing T cell functional activity are known in the art.

In certain embodiments the compounds of Formula I in accordance with the present invention are used simultaneously, in the same formulation or in separate formulations, or sequentially with an additional agent(s) as part of a combination therapy regimen.

Therapeutically effective dosages of a compound according to Formula (I) or a composition of a Formula (I) compound will generally range from about 1 to 2000 mg/day, from about 10 to about 1000 mg/day, from about 10 to about 500 mg/day, from about 10 to about 250 mg/day, from about 10 to about 100 mg/day, or from about 10 to about 50 mg/day. The therapeutically effective dosages may be administered in one or multiple doses. It will be appreciated, however, that specific doses of the compounds of the invention for any particular patient will depend on a variety of factors such as age, sex, body weight, general health condition, diet, individual response of the patient to be treated, time of administration, severity of the disease to be treated, the activity of particular compound applied, dosage form, mode of application and concomitant medication. The therapeutically effective amount for a given situation will readily be determined by routine experimentation and is within the skills and judgment of the ordinary clinician or physician. In any case the compound or composition will be administered at dosages and in a manner which allows a therapeutically effective amount to be delivered based upon patient's unique condition.

### General Synthetic Schemes

R¹, R², X¹, Y¹ and n are previously defined for Formula I and these definitions are utilized in the schemes below.

Compounds of Formula I can be prepared from symmetrical biphenyl intermediates III where R^{1a} is CH₃, CH₂OH, CH₂OPG, CHO, CO₂R² and PG is a Protecting Group.

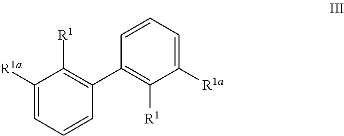

Symmetrical biphenyl can be synthesized by starting with commercially or readily available benzene derivatives II employing one of several methods described in the literature.

Comprehensive reviews of biphenyls include Jain, Z. J., et al. ArJChem. 2017 10, S2051, Hassan, Chem. Rev., 2002, 102, pg. 1359, Bringmann, Angew. Chem. Int. Ed., 1990, 29, pg. 977 and Sainsbury, Tetrahedron, 1980, 36, pg. 3327.

In one approach symmetrical biphenyls of Formula III can be directly formed by the dimerization of II via metal catalysis where Lv is a Leaving Group such as halogen, OSO₂CH₃, OSO₂Ar or OSO₂CF₃.

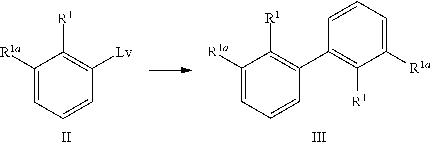

In a second synthetic approach symmetrical biphenyls of Formula III can be synthesized via a Suzuki-Miyaura reaction (see Miyaura, N., Suzuki, A. Chem. Rev. 1995, 95, 2457., Kotha, S. et al. Tetrahedron 2002, 58, 9633, Broutin, P., et al., Org. Lett. 2004, 6 (24), 4419 and Molander, G. A., et al., Aldrichimica Acta 2005, 38, 49). In this versatile method, benzene derivatives II can first be converted to a boron species IIA where B is a boron species such as B(OH)₂, B(O-alkyl)₂, BF₃K, or Bpin.

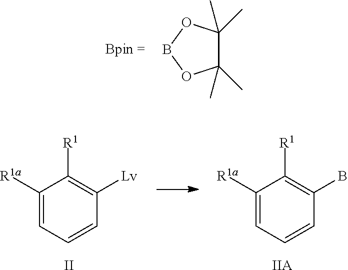

Benzene derivative II can them be subjected to palladium catalyzed conditions with boron species IIA to form biphenyls of Formula III.

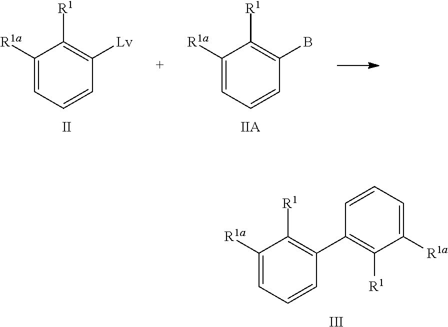

To further transform intermediate biphenyls of Formula III to the biphenyl bis-ethers of Formula IV, the R^{1a} group must first be converted to a hydroxymethylene moiety if not already in place. Intermediate IIIA (R^{1a} is CH₂OH) can then be subjected to an ether forming reaction with an appropriate phenol under Mitsunobu conditions to form biphenyl bis-ethers of Formula IV where R^{1b} is CH₂OH, CH₂OPG, CHO, CO₂R², CH₂NH₂ or CH₂NHPG. For a review of the Mitsunobu reaction see Fletcher, S., Org. Chem. Front. 2015, 2, 739.

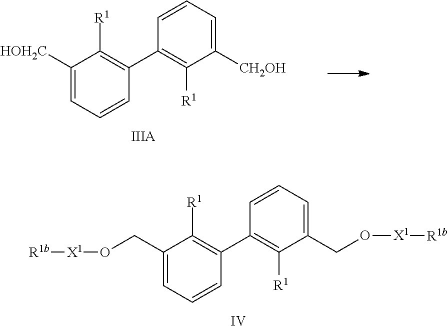

Alternatively IIIA may first be converted to IIIB which can then be combined with an appropriate phenol under anionic nucleophilic reaction conditions including sodium hydride in anhydrous tetrahydrofuran and potassium carbonate in acetone to form biphenyl bis-ethers of Formula IV.

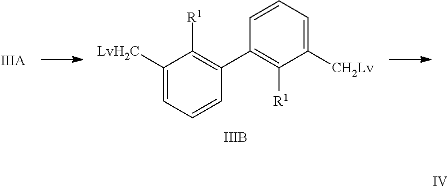

Functional group R^{1b} of intermediate IV can either then be used to directly incorporate the substituted alkyl groups designated as (CH₂)ₙY¹ of Formula I, or first converted to an alternative functional group required to transform IV to I. For example when R^{1b} is an aldehyde it may be subjected to reductive alkylation or carbon homologation transformations.

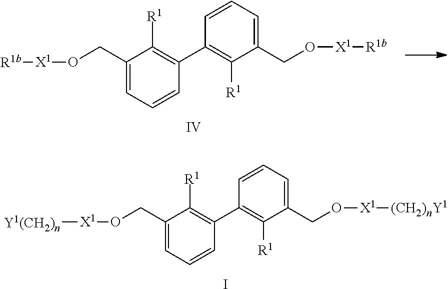

### EXAMPLES

All synthetic chemistry was performed in standard laboratory glassware unless indicated otherwise in the examples. Commercial reagents were used as received. Microwave reactions were performed in a Biotage Initiator using the instrument software to control heating time and pressure. Analytical LC/MS was performed on an Agilent 1290 infinity, Mass:6150 SQD(ESI/APCI) or an Agilent 1200 SERIES, Mass:6130SQD(ESI/APCI); variable wavelength detector and Agilent 6130 single quadrupole mass spectrometer, alternating positive and negative ion scans using Chemistation software. Retention times were determined from the extracted 220 nm UV chromatogram. HPLC was performed on a Waters 2695 system with a variable wavelength detector using Empower software. Retention times were determined from the extracted 210 nm and 300 nm UV chromatograms. ¹H NMR was performed on a Bruker Avance 400 at 400 MHz or a Bruker Avance DRX-500 at 500 MHz using Topspin software. For complicated splitting patterns, the apparent splitting was tabulated. Analytical thin layer chromatography was performed on silica (Macherey-Nagel ALUGRAM Xtra SIL G, 0.2 mm, UV₂₅₄ indicator) and was visualized under UV light. Silica gel chromatography was performed manually, or with Grace automated chromatography for gradient elution. Melting points were collected using a Biichi B-540 melting point apparatus.

### Example 1

### (2S,2′S)-1,1′-(((((2,2′-dimethyl-[1,1′-biphenyl]-3,3′-diyl)bis(methylene))bis(oxy))bis(3-bromo-4,1-phenylene))bis(methylene))bis(piperidine-2-carboxylic acid) (Cpd 1)

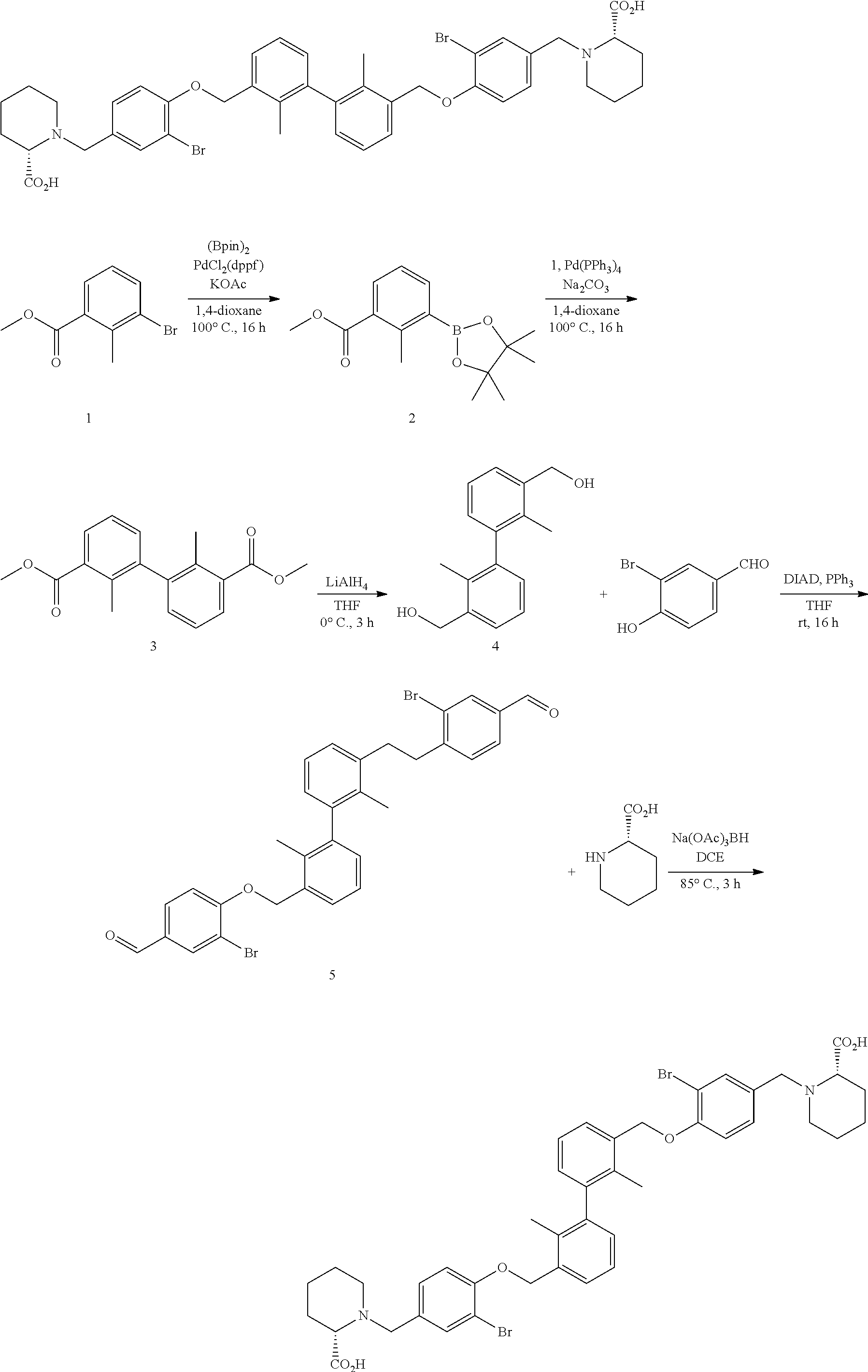

### Step 1

### Methyl 2-methyl-3-(4,4,5,5-tetramethyl-1,3,2-dioxaborolan-2-yl)benzoate (2)

PdCl₂(dppf). CH₂Cl₂ (80 g, 21 mmol) was added to a de-gassed solution of methyl 3-bromo-2-methylbenzoate (100 g, 438 mmol) and bis(pinacolato)diboran (89 g, 400 mmol) and KOAc (107.4 g, 1.095 mol) in 1,4-dioxane (600 mL) and stirred at 100° C. for 16 h. The solvent was removed by rotary evaporation. The residue was diluted with ice-cold water (100 mL) and the aqueous layer was extracted with ethyl acetate (2×1 L), washed with water (500 mL) and brine (500 mL), dried over anhydrous Na₂SO₄, filtered and concentrated under reduced pressure to afford methyl 2-methyl-3-(4,4,5,5-tetramethyl-1,3,2-dioxaborolan-2-yl)benzoate 2 (120 g) as a black gummy solid. The crude product was used in the next step without purification. ES+, m/z 277.1 [M+H]; [C₁₅H₂₁BO₄]; ¹H NMR (400 MHz, CDCl₃): δ 7.86-7.82 (m, 2H), 7.27-7.19 (m, 1H), 3.89 (s, 3H), 2.73 (s, 3H), 1.35 (s, 12H).

### Step 2

### Dimethyl 2,2′-dimethylbiphenyl-3,3′-dicarboxylate (3)

To a solution of methyl 3-bromo-2-methylbenzoate 1 (65 g, 285 mmol) and methyl 2-methyl-3-(4,4,5,5-tetramethyl-1,3,2-dioxaborolan-2-yl)benzoate 2 (118.5 g, 427 mmol) in 1,4-dioxane (800 mL) and water (200 mL) was added Na₂CO₃ (90.6 g, 855 mmol) and the reaction mixture was purged with argon gas for 10 min, prior to the addition of Pd(PPh₃)₄(23 g, 19.9 mmol) and the reaction mixture was degassed again for 10 min. The reaction mixture was stirred at 85° C. for 16 h. The reaction mixture was poured into ice cold water and extracted with ethyl acetate (2×1 L). The combined organic layer was dried over Na₂SO₄ and concentrated under reduced pressure. The crude compound was purified by silica gel (100-200 mesh) column chromatography eluting with 0-10% ethyl acetate in pet ether as a solvent to afford dimethyl 2,2′-dimethylbiphenyl-3,3′-dicarboxylate 3 (66 g, 77%) as a pale yellow solid. ES+, m/z 299.1 [M+H]; [C₁₈H₁₈O₄]; ¹H NMR (400 MHz, CDCl₃): δ 7.86-7.84 (dd, J=7.6, 1.2 Hz, 2H), 7.30-7.21 (m, 4H), 3.91 (s, 6H), 2.27 (s, 6H).

### Step 3

### (2,2′-Dimethylbiphenyl-3,3′-diyl)dimethanol (4)

To a stirred solution of LAH (327 mL, 654 mmol, 2.0 M solution in THF) in THF (1 L) at 0° C. was added drop-wise a solution of dimethyl 2,2′-dimethylbiphenyl-3,3′-dicarboxylate 3 (65 g, 218 mmol) in THF (300 mL) at 0° C. The resulting reaction mixture was stirred at 0° C. for 3 h. The reaction mixture was quenched with sat. NH₄Cl solution and extracted with ethyl acetate (2×1 L). The combined organic layer was dried over Na₂SO₄ and concentrated under reduced pressure. The crude compound was purified by silica gel (100-200 mesh) column chromatography eluting with 0-35% ethyl acetate in pet ether as solvent to afford (2,2′-dimethylbiphenyl-3,3′-diyl)dimethanol 4 (40 g, 75%) as an off-white solid. ES+, m/z 265.1 [M+23]; [C₁₆H₁₈O₂]; ¹H NMR (400 MHz, CDCl₃): δ 7.39-7.38 (d, J=6.8 Hz, 2H), 7.26-7.22 (m, 2H), 7.08-7.06 (dd, J=7.6, 1.2 Hz, 2H), 4.77-4.76 (d, J=5.6 Hz, 4H), 2.03 (s, 6H), 1.62-1.59 (t, J=5.6 Hz, 2H).

### Step 4

### 4,4′-(((2,2′-Dimethyl-[1,1′-biphenyl]-3,3′-diyl)bis(methylene))bis(oxy))bis(3-bromobenzaldehyde) (5)

To a solution of (2,2′-dimethylbiphenyl-3,3′-diyl)dimethanol (30 g, 123.96 mmol) in THF (750 mL) were added 3-bromo-4-hydroxybenzaldehyde (49.46 g, 247.93 mmol) and triphenylphosphine (81.19 g, 309.91 mmol) at 0° C., followed by DIAD (62.6 g, 309.91 mmol) and the reaction mixture was stirred at room temperature for 16 h. The reaction mixture was poured into ice-cold water and extracted with ethyl acetate (2×500 mL). The combined organic layer was dried over Na₂SO₄ and concentrated under reduced pressure. The resulting crude compound was purified by silica gel (100-200 mesh) column chromatography eluting with 0-20% ethyl acetate in pet ether as solvent to afford 4,4′-(((2,2′-dimethyl-[1,1′-biphenyl]-3,3′-diyl)bis(methylene))bis(oxy))bis(3-bromobenzaldehyde) 5 (18 g, 24%) as an off white solid. [C₃₀H₂₄Br₂O₄]; ¹H NMR (400 MHz, CDCl₃): δ 9.86 (s, 2H), 8.13-8.12 (d, J=1.6 Hz, 2H), 7.85-7.82 (dd, J=6.0, 2.4 Hz, 2H), 7.54-7.52 (d, J=7.4 Hz, 2H), 7.32-7.29 (t, J=7.2 Hz, 2H), 7.19-7.13 (m, 4H), 5.27 (s, 4H), 2.08 (s, 6H).

### Step 5

### (2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(piperidine-2-carboxylic acid

To a solution of 4,4′-(2,2′-dimethylbiphenyl-3,3′-diyl)bis(methylene)bis(oxy)bis(3-bromobenzaldehyde) 5 (18 g, 29.70 mmol) in 1,2-dichloroethane (360 mL) were added sodium triacetoxyborohydride (31.33 g, 148.51 mmol) and (S)-piperidine-2-carboxylic acid (19.15 g, 148.51 mmol). The reaction mixture was stirred at room temperature for 20 min and then at 85° C. for 3 h. The reaction mixture was poured into ice cold water. Solid thus precipitated was filtered and dried under vacuum to get crude compound. The crude compound was purified by GRACE flash chromatography (20% MeOH—CH₂Cl₂, silica gel) to afford (2S,2′S)-1,1′-(((((2,2′-dimethyl-[1,1′-biphenyl]-3,3′-diyl)bis(methylene))bis(oxy))bis(3-bromo-4,1-phenylene))bis(methylene))bis(piperidine-2-carboxylic acid) (5.6 g, 23%) as an off-white solid. ES−, m/z 830.9 [M−H]; [C₄₂H₄₆Br₂N₂O₆]; ¹H NMR (400 MHz, DMSO-d₆): δ 7.57 (s, 2H), 7.54 (d, J=7.6 Hz, 2H), 7.32-7.24 (m, 6H), 7.11 (d, J=7.2 Hz, 2H), 5.23 (s, 4H), 3.81 (d, J=13.2 Hz, 2H), 3.45 (d, J=14.0 Hz, 2H), 3.08-3.06 (m, 2H), 2.87-2.85 (m, 2H), 2.24-2.02 (m, 2H), 2.02 (s, 6H), 1.83-1.72 (m, 2H), 1.69-1.67 (m, 2H), 1.51-1.46 (m, 6H), 1.37-1.32 (m, 2H). m.p.=222-226° C.

### Example 2

### (2R,2′R)-1,1′-(((((2,2′-dimethyl-[1,1′-biphenyl]-3,3′-diyl)bis(methylene))bis(oxy))bis(3-bromo-4,1-phenylene))bis(methylene))bis(piperidine-2-carboxylic acid) (Cpd 2)

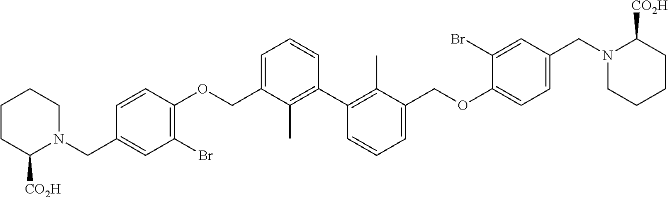 [SMILES: Cc1c(COc2ccc(CN3CCCC[C@@H]3C(=O)O)cc2Br)cccc1-c1cccc(COc2ccc(CN3CCCC[C@@H]3C(=O)O)cc2Br)c1C]

Using conditions describe in Example 1, (2R,2′R)-1,1′-(((((2,2′-dimethyl-[1,1′-biphenyl]-3,3′-diyl)bis(methylene))bis(oxy))bis(3-bromo-4,1-phenylene))bis(methylene))bis(piperidine-2-carboxylic acid) was prepared from 4,4′-(2,2′-dimethylbiphenyl-3,3′-diyl)bis(methylene)bis(oxy)bis(3-bromobenzaldehyde) and (R)-piperidine-2-carboxylic acid. The crude compound was purified by prep-HPLC (Mobile phase: 0.1% TFA in H₂O: ACN, Column: KROMOSIL-C18 (150*25), 10u, Gradient: (T % B): 0/10, 6.3/55, 6.4/98, 8/98, 8.1/10, 10/10, Flow Rate: 25 ml/min, Diluent: ACN+H₂O+MeOH+THF) to afford (2R,2′R)-1,1′-(((((2,2′-dimethyl-[1,1′-biphenyl]-3,3′-diyl)bis(methylene))bis(oxy))bis(3-bromo-4,1-phenylene))bis(methylene))bis(piperidine-2-carboxylic acid) (250 g, 20%) as an off-white solid. ES+, m/z 833.49 [M+H]; [C₄₂H₄₆Br₂N₂O₆]; ¹H NMR (400 MHz, DMSO-d₆); δ 7.56-7.52 (m, 4H), 7.31-7.23 (m, 6H), 7.10 (d, J=7.2 Hz, 2H), 5.23 (s, 4H), 3.81 (d, J=5.2 Hz, 2H), 3.42 (d, J=13.2 Hz, 2H), 3.16 (s, 2H), 3.05-3.04 (m, 2H), 2.86-2.83 (m, 2H), 2.11 (bs, 2H), 2.02 (s, 6H), 1.82-1.75 (m, 2H), 1.72-1.64 (m, 2H), 1.58-1.47 (m, 6H), 1.41-1.31 (m, 2H).

### UPLC Conditions

### Column: ACquity UPLC BEH C18 (2.1×100) mm, 1.7 μm

Mobile phase: 0.05% TFA in H₂O, B: 0.05% TFA in Acetonitrile
Flow rate: 0.3 mL/min
T/% B (min): 0/3, 4/90, 6/90, 6.1/30.

### Diluent: ACN

### Temp: 25° C.

Ret. time: 2.45 min

### Purity: 99.47%.

### Example 3

### N,N′-(((((((2,2′-Dimethyl-[1,1′-biphenyl]-3,3′-diyl)bis(methylene))bis(oxy))bis(2-methoxypyridine-6,3-diyl))bis(methylene))bis(azanediyl))bis(ethane-2,1-diyl))diacetamide (Cpd 3)

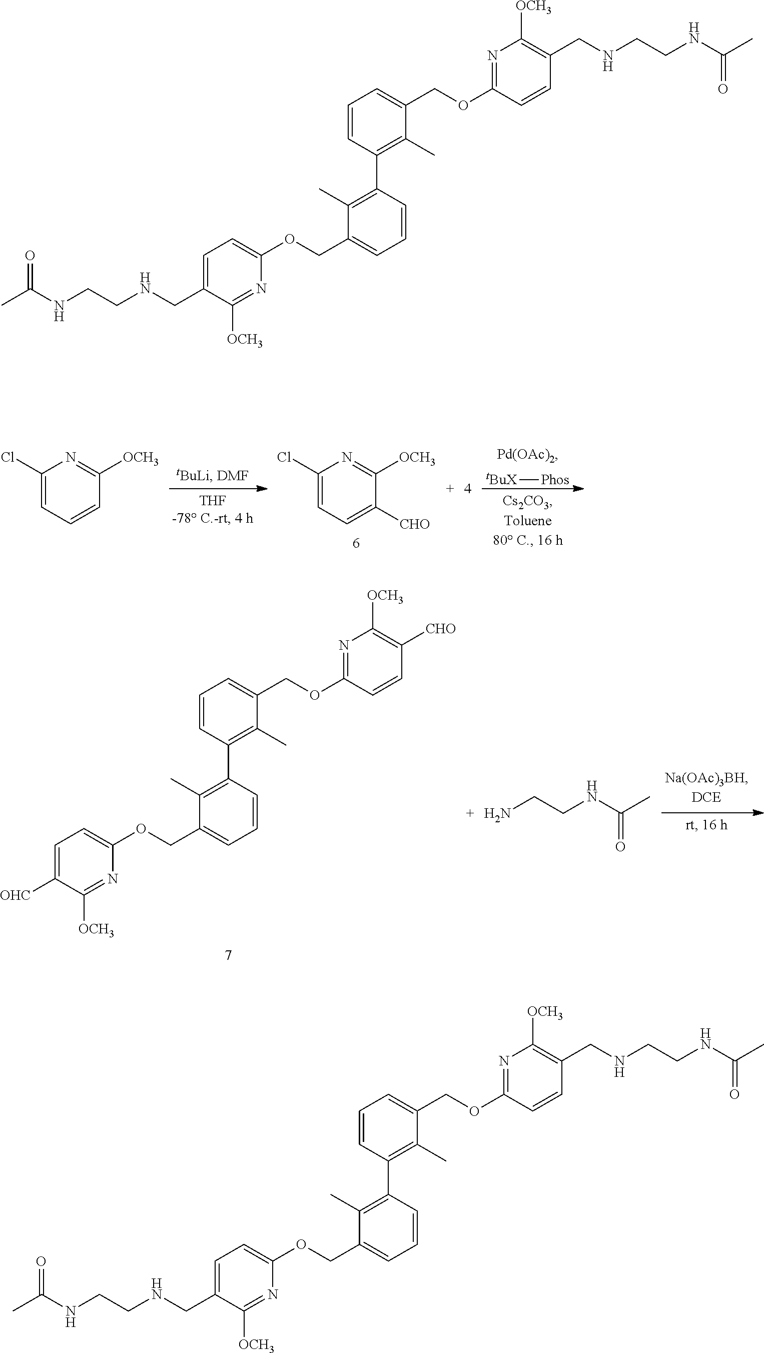

### Step 1

### 6-Chloro-2-methoxynicotinaldehyde (6)

To a solution of 2-chloro-6-methoxypyridine (5 g, 34.82 mmol) in THF (100 mL), cooled to −78° C. was added ^{t}BuLi (1.7 M) (18.5 mL, 31.34 mmol) and stirred for 1 h; DMF (92.2 g, 31.34 mmol) was then added at −78° C. and stirred for 2 h. The reaction mixture was quenched with acetic acid and poured in to ice cold water, basified with sat NaHCO₃ and extracted with EtOAc (2×500 mL). The combined organic layer was dried over Na₂SO₄ and concentrated under reduced pressure to get crude product, which was purified by silica gel column (eluting with pet ether: ethyl acetate=10:1) to afford 6-chloro-2-methoxynicotinaldehyde as an off white solid (3.8 g, 65%). ES+, m/z 172 [M+H]; [C₇H₆ClNO₂], ¹H NMR (300 MHz, CDCl₃) δ 10.17 (s, 1H), 8.07 (d, J=8.04 Hz, 1H), 7.03 (d, J=8.04 Hz, 1H), 3.79 (s, 3H).

### Step 2

### 6,6′-(((2,2′-Dimethyl-[1,1′-biphenyl]-3,3′-diyl)bis(methylene))bis(oxy))bis(2-methoxynicotinaldehyde) (7)

To a solution of aldehyde 6 (2.11 g, 12.39 mmol) and (2,2′-dimethylbiphenyl-3,3′-diyl)dimethanol 4 (1.5 g, 6.19 mmol) in toluene (25 mL) was added and Cs₂CO₃ (6.04 g, 18.57 mmol) and purged with argon gas for 10 min. To this reaction mixture were added Pd(OAc)₂ (140 mg, 0.61 mmol) and ^{t}BuX-Phos (520 mg, 1.23 mmol) and degassed for 10 min and then the reaction mass was stirred at 80° C. for 16 h. The reaction mixture was filtered on celite pad. The filtrate was evaporated under reduced pressure and the crude compound was purified by silica gel column chromatography (eluting with pet-ether: ethyl acetate=8:2) to afford 6,6′-(((2,2′-dimethyl-[1,1′-biphenyl]-3,3′-diyl)bis(methylene))bis(oxy))bis(2-methoxynicotinaldehyde) (1.85 g, 55%) as an off white solid. ES+, m/z 535 [M+23]; [C₃₀H₂₈N₂O₆]; ¹HNMR (400 MHz, CDCl₃); δ 10.09 (s, 2H), 8.05 (d, J=8.3 Hz, 2H), 7.42 (d, J=7.34 Hz, 2H), 7.21-7.28 (m, 2H), 7.12 (d, J=6.8 Hz, 2H), 6.44 (d, J=8.3 Hz, 2H), 5.38-5.61 (m, 4H), 4.11 (s, 6H), 2.12 (s, 6H).

### Step 3

### N,N′-(((((((2,2′-Dimethyl-[1,1′-biphenyl]-3,3′-diyl)bis(methylene))bis(oxy))bis(2-methoxypyridine-6,3-diyl))bis(methylene))bis(azanediyl))bis(ethane-2,1-diyl))diacetamide

To a solution of 6,6′-(((2,2′-dimethyl-[1,1′-biphenyl]-3,3′-diyl)bis(methylene))bis(oxy))bis(2-methoxynicotinaldehyde) (750 mg, 1.46 mmol) in 1,2-dichloroethane (10 mL) was added sodium triacetoxyborohydride (1.23 g, 5.84 mmol) and N-(2-aminoethyl)acetamide (740 mg, 7.32 mmol). The reaction mixture was stirred at room temperature for 20 min then at 85° C. for 3 h. The reaction mixture was poured in to ice cold water and acidified with 1N HCl to pH ˜3 and extracted with CH₂Cl₂. The combined organic layer was dried over Na₂SO₄ and concentrated under reduced pressure. The crude compound was purified by prep-HPLC to afford N,N′-(((((((2,2′-dimethyl-[1,1′-biphenyl]-3,3′-diyl)bis(methylene))bis(oxy))bis(2-methoxypyridine-6,3-diyl))bis(methylene))bis(azanediyl))bis(ethane-2,1-diyl))diacetamide (230 mg, 33%) as a colorless gum. ES+, m/z 685.64 [M+H]; [C₃₈H₄₈N₆O₆], ¹H NMR (400 MHz, DMSO-d₆) δ 7.78-7.71 (m, 2H), 7.61 (d, J=10.8 Hz, 2H), 7.44 (d, J=7.6 Hz, 2H), 7.25 (t, J=10.4 Hz, 2H), 7.05 (d, J=9.2 Hz, 2H), 6.42 (d, J=10.8 Hz, 2H), 5.40 (s, 4H), 3.86 (s, 6H), 3.55 (s, 4H), 3.13-3.07 (m, 4H), 2.47 (m, 4H), 2.01 (s, 6H), 1.77 (s, 6H).

### UPLC Conditions

### Column: Acquity UPLC BEH C18 (2.1×100) mm, 1.7 μm

Mobile phase: 0.05% TFA in H₂O, B: 0.05% TFA in Acetonitrile
Flow rate: 0.3 mL/min
T/% B (min): 0/10, 4/90, 6/90, 6.1/10.

### Diluent: ACN

### Temp.: 25° C.

Ret. time: 2.81 min

### Purity: 96.8%.

### Example 4

### Dimethyl 1,1′-(((((2,2′-dimethyl-[1,1′-biphenyl]-3,3′-diyl)bis(methylene))bis(oxy))bis(3-bromo-4,1-phenylene))bis(methylene))(2S,2′S)-bis(piperidine-2-carboxylate) (Cpd 4)

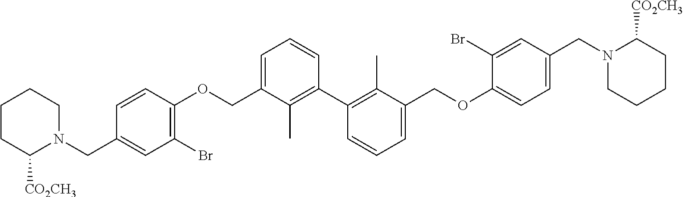 [SMILES: COC(=O)[C@@H]1CCCCN1Cc1ccc(OCc2cccc(-c3cccc(COc4ccc(CN5CCCC[C@H]5C(=O)OC)cc4Br)c3C)c2C)c(Br)c1]

To a solution of (2S,2′S)-1,1′-(((((2,2′-dimethyl-[1,1′-biphenyl]-3,3′-diyl)bis(methylene))bis(oxy))bis(3-bromo-4,1-phenylene))bis(methylene))bis(piperidine-2-carboxylic acid) (Cpd 1) (300 mg, 0.359 mmol) in methanol (3 mL) and dichloromethane (3 mL) was added trimethylsilyl diazomethane (6 mL) at 0° C. The reaction mixture was stirred at 0° C. for 1 h. The reaction mixture was diluted with water (20 mL) and extracted with dichloromethane (2×20 mL). The combined CH₂Cl₂ layer was washed with brine (20 mL), dried over Na₂SO₄ and concentrated under vacuum. The crude compound was purified by GRACE flash chromatography (30% ethyl acetate in pet-ether, silica gel) to afford dimethyl 1,1′-(((((2,2′-dimethyl-[1,1′-biphenyl]-3,3′-diyl)bis(methylene))bis(oxy))bis(3-bromo-4,1-phenylene))bis(methylene))(2S,2′S)-bis(piperidine-2-carboxylate) (220 mg, 71%) as an off-white gummy solid. ES+, m/z 861.2 [M+H]; [C₄₄H₅₀Br₂N₂O₆]; ¹H NMR (400 MHz, DMSO-d₆): δ 7.54-7.52 (d, J=7.2 Hz, 2H), 7.50 (s, 2H), 7.32-7.38 (t, J=7.6 Hz, 2H), 7.24-7.21 (m, 4H), 7.11-7.09 (d, J=6.8 Hz, 2H), 5.22 (s, 4H), 3.64 (m, 6H), 3.63-3.59 (d, J=14.4 Hz, 2H), 3.34-3.31 (d, J=13.6 Hz, 2H), 3.21-3.18 (m, 2H), 2.80-2.77 (m, 2H), 2.16-2.13 (m, 2H), 2.02 (s, 6H), 1.74-1.69 (m, 4H), 1.48-1.36 (m, 8H).

### Example 5

### 2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(ethan-1-ol) di-trifluoroacetic acid salt (Cpd 5)

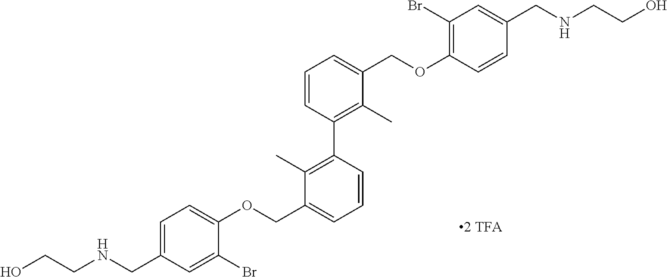 [SMILES: Cc1c(COc2ccc(CNCCO)cc2Br)cccc1-c1cccc(COc2ccc(CNCCO)cc2Br)c1C]

To a solution of 4,4′-(((2,2′-dimethyl-[1,1′-biphenyl]-3,3′-diyl)bis(methylene))bis(oxy))bis(3-bromobenzaldehyde) 5 (300 mg, 0.495 mmol) in 1,2-dichloroethane (20 mL) were added 2-aminoethanol (0.14 mL, 2.475 mmol) and sodium triacetoxyborohydride (524 mg, 2.475 mmol) The reaction mixture was stirred at room temperature for 20 min then at 85° C. for 5 h. The reaction mixture was poured in to ice-cold water (50 mL) and extracted with ethyl acetate (100 mL×3). The combined extract was washed with brine, dried over Na₂SO₄, and concentrated to give the crude neutral product, which was purified under acidic conditions by GRACE flash chromatography (20% MeOH—CH₂Cl₂, silica gel) to afford 2,2′-((((((2,2′-dimethyl-[1,1′-biphenyl]-3,3′-diyl)bis(methylene))bis(oxy))bis(3-bromo-4,1-phenylene))bis(methylene))bis(azanediyl))bis(ethan-1-ol) (90 mg, as TFA salt, yield: 26%, HPLC 98.4%) as a white solid. ES+, m/z 696.7 [M+H]; [C₃₄H₃₈Br₂N₂O₄]; ¹H NMR (400 MHz, DMSO-d₆) δ 7.55-7.48 (m, 4H), 7.31-7.27 (m, 3H), 7.23-7.17 (m, 4H), 7.19 (d, J=7.2 Hz, 2H), 5.21 (d, J=7.2 Hz, 4H), 4.45 (bs, 1H), 4.29 (s, 2H), 3.63 (s, 2H), 3.44 (m, 2H), 3.31 (s, 2H), 3.11-3.10 (m, 2H), 2.52 (s, 2H), 2.02 (s, 6H). m.p.=136-140° C.

### Example 6

### N,N′-(((((((2,2′-Dimethyl-[1,1′-biphenyl]-3,3′-diyl)bis(methylene))bis(oxy))bis(3-bromo-4,1-phenylene))bis(methylene))bis(azanediyl))bis(ethane-2,1-diyl))diacetamide (Cpd 6)

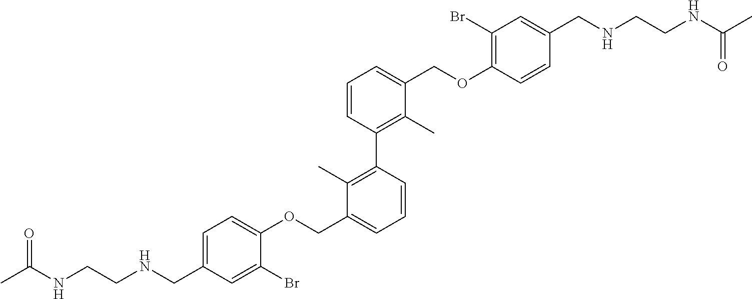 [SMILES: CC(=O)NCCNCc1ccc(OCc2cccc(-c3cccc(COc4ccc(CNCCNC(C)=O)cc4Br)c3C)c2C)c(Br)c1]

To a solution of 4,4′-(((2,2′-dimethyl-[1,1′-biphenyl]-3,3′-diyl)bis(methylene))bis(oxy))bis(3-bromobenzaldehyde) 5 (400 mg, 0.66 mmol) in DMF (15 mL) were added N-(2-aminoethyl)acetamide (0.33 mg, 3.30 mmol) and sodium triacetoxyborohydride (699 mg, 3.30 mmol). The reaction mixture was stirred at room temperature for 5 h. The reaction mixture was poured into ice-cold water (50 mL) and extracted with 10% CH₂Cl₂ in MeOH (3×50 mL). The combined extract was washed with brine, dried over Na₂SO₄, and concentrated to give the crude product, which was purified by prep-HPLC (Mobile phase: 10 mM Ammonium bicarbonate in H₂O:ACN; Column: KROMASIL-C18 (25*150 mm),10u; Gradient:(T % B):—0/50, 8/70, 8.1/98, 10/98, 10.1/50, 12/50; Flow Rate: 25 ml/min; Diluent:ACN+H₂O+THF+MeOH) to afford N,N′-(((((((2,2′-dimethyl-[1,1′-biphenyl]-3,3′-diyl)bis(methylene))bis(oxy))bis(3-bromo-4,1-phenylene))bis(methylene))bis(azanediyl))bis(ethane-2,1-diyl))diacetamide (80 mg, yield: 15%, HPLC 96.38%) as a white solid. ES+, m/z 779.0 [M+H]; [C₃₈H₄₄Br₂N₄O₄]; ¹H NMR (400 MHz, DMSO-d₆) δ 7.80-7.72 (m, 2H), 7.51 (d, J=7.2 Hz, 2H), 7.34-7.30 (m, 4H), 7.24 (d, J=8.0 Hz, 2H), 7.11 (d, J=6.8 Hz, 2H), 5.22 (s, 4H), 3.61 (s, 4H), 3.11 (t, J=6.4 Hz, 4H), 2.49-2.47 (m, 4H), 2.03 (s, 6H), 1.78 (s, 6H). m.p.=95-99° C.

### Example 7

### (2S,2'S,4S,4′S)-1,1′-(((((2,2′-Dimethyl-[1,1′-biphenyl]-3,3′-diyl)bis(methylene))bis(oxy))bis(3-bromo-4,1-phenylene))bis(methylene))bis(4-hydroxypyrrolidine-2-carboxylic acid) (Cpd 7)

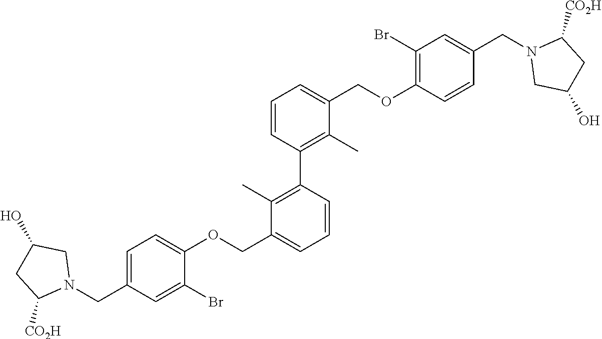 [SMILES: Cc1c(COc2ccc(CN3C[C@@H](O)C[C@H]3C(=O)O)cc2Br)cccc1-c1cccc(COc2ccc(CN3C[C@@H](O)C[C@H]3C(=O)O)cc2Br)c1C]

To a solution of 4,4′-(((2,2′-dimethyl-[1,1′-biphenyl]-3,3′-diyl)bis(methylene))bis(oxy))bis(3-bromobenzaldehyde) 5 (300 mg, 0.495 mmol) in MeOH (20 mL) were added (2S,4S)-4-hydroxypyrrolidine-2-carboxylic acid (324.57 mg, 2.47 mmol) and AcOH (3-4 drops) and stirred at 80° C. for 4 h. The reaction mixture was cooled to room temperature, prior to the addition of NaCNBH₃ (155.54 mg, 3.30 mmol). The reaction mixture was stirred at 80° C. for 3 h. The reaction mixture was poured in to ice cold water and solid thus precipitated was filtered and dried under vacuum to get crude compound. The crude compound was purified by GRACE flash chromatography (20% MeOH—CH₂Cl₂, silica gel) to afford (2S,2'S,4S,4′S)-1,1′-(((((2,2′-dimethyl-[1,1′-biphenyl]-3,3′-diyl)bis(methylene))bis(oxy))bis(3-bromo-4,1-phenylene))bis(methylene))bis(4-hydroxypyrrolidine-2-carboxylic acid) (45 mg, yield: 13%, HPLC 99.10%) as an off white solid. ES−, m/z 836.8 [M−H]; [C₄₀H₄₂Br₂N₂O₈]; ¹H NMR (500 MHz, DMSO-d₆) δ 7.54-7.52 (m, 4H), 7.31 (m, 4H), 7.19-7.18 (m, 2H), 7.10 (d, J=7.5 Hz, 2H), 5.21 (s, 4H), 4.01 (s, 2H), 3.78 (d, J=13.5 Hz, 2H), 3.54 (d, J=13.5 Hz, 2H), 3.19 (d, J=6.5 Hz, 2H), 2.63-2.61 (m, 4H), 2.02-1.97 (m, 8H), 1.66 (d, J=13.0 Hz, 2H). m.p.=260-264° C.

### Example 8

### (2S,2′S)-1,1′-(((([1,1′-Biphenyl]-3,3′-diylbis(methylene))bis(oxy))bis(3-bromo-4,1-phenylene))bis(methylene))bis(piperidine-2-carboxylic acid) (Cpd 8)

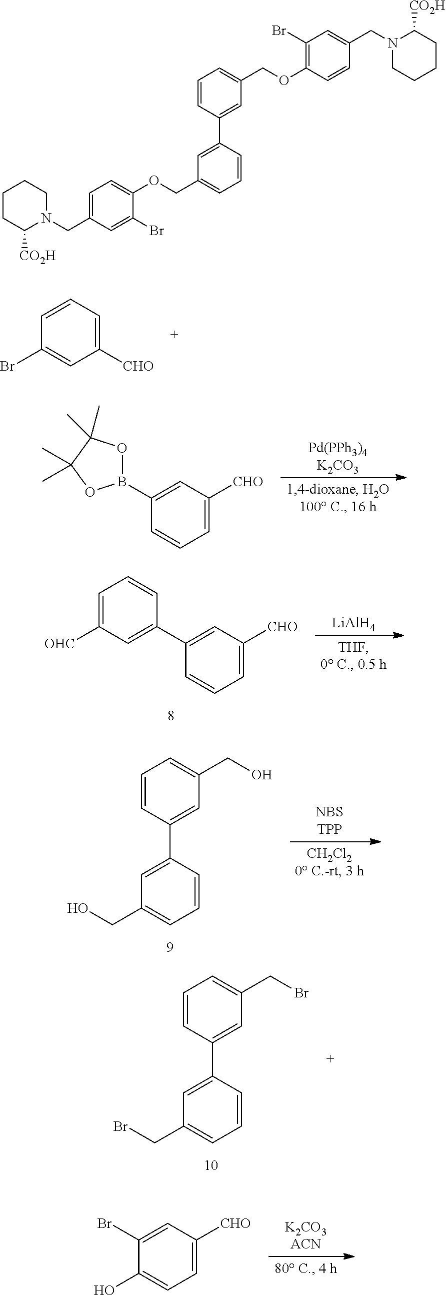

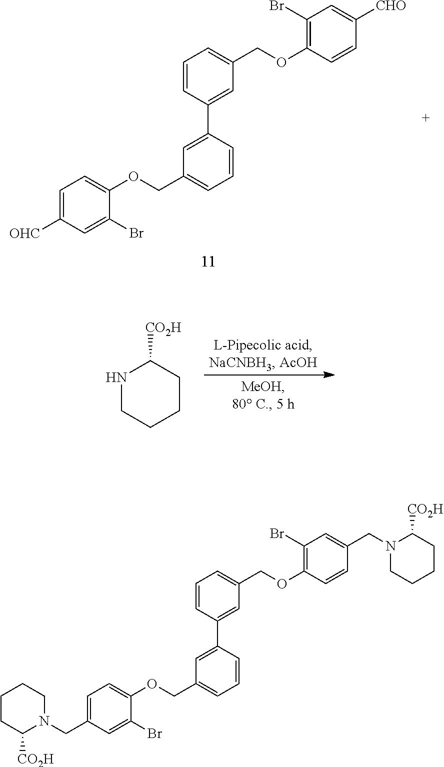 [SMILES: O=Cc1ccc(OCc2cccc(-c3cccc(COc4ccc(C=O)cc4Br)c3)c2)c(Br)c1 ; O=C(O)[C@@H]1CCCCN1 ; O=C(O)[C@@H]1CCCCN1Cc1ccc(OCc2cccc(-c3cccc(COc4ccc(CN5CCCC[C@H]5C(=O)O)cc4Br)c3)c2)c(Br)c1]

### Step 1

### Biphenyl-3,3′-dicarbaldehyde (8)

To a solution of 3-bromobenzaldehyde (6 g, 34.654 mmol) and 3-(4,4,5,5-tetramethyl-1,3,2-dioxaborolan-2-yl)benzaldehyde (5.1 g, 34.654 mmol) in 1,4-dioxane (40 mL) and water (10 mL) was added K₂CO₃ (9.39 g, 68.108 mmol) and the reaction mixture was purged with argon for 10 min, prior to the addition of Pd(PPh₃)₄(3.9 g, 3.465 mmol) and again degassed for 10 min. The reaction mixture was stirred at 85° C. for 16 h. The reaction mixture was poured into ice-cold water and extracted with ethyl acetate (2×1 L). The combined organic layer was dried over Na₂SO₄ and concentrated to give the crude product, which was purified on a silica gel column, eluting with ethyl acetate in pet ether (0.20%) to get biphenyl-3,3′-dicarbaldehyde 8 (6 g, 88%) as an off-white solid, [C₁₄H₁₀O₂]; ¹H NMR (500 MHz, CDCl₃) δ 10.05 (s, 2H), 8.15-8.14 (m, 2H), 7.93-7.90 (m, 4H), 7.67 (t, J=9.5 Hz, 2H).

### Step 2

### Biphenyl-3,3′-diyldimethanol (9)

To a stirred solution of biphenyl-3,3′-dicarbaldehyde (6 g, 28.5 mmol) in MeOH (100 mL) at 0° C. was added NaBH₄ (5.28 g, 142.85 mmol) portion wise at 0° C. The resulting reaction mixture was stirred at 0° C. for 2 h. The reaction mixture was quenched with sat.NH₄Cl and extracted with ethyl acetate (2×1 L). The combined organic layer was dried over Na₂SO₄ and concentrated to give the crude product, which was purified on a silica gel column, eluting with ethyl acetate in pet ether (0-35%) to get biphenyl-3,3′-diyldimethanol (5.9 g, 96%, LC/MS-91%) as an off white solid. [C₁₄H₁₄O₂]; ¹H NMR (400 MHz, CDCl₃) δ 7.61 (s, 2H), 7.53 (d, J=9.0 Hz, 2H), 7.44 (t, J=9.0 Hz, 2H), 7.35 (d, J=9.0 Hz, 2H), 4.77-4.76 (m, 4H).

### Step 3

### 3,3′-Bis(bromomethyl)biphenyl (10)

To a solution of biphenyl-3,3′-diyldimethanol (4.9 g, 22.897 mmol) and Ph₃P (17.9 g, 68.6 mmol, 3 eq.) in CH₂Cl₂ (100 mL) at 0° C., NBS (16.3 g, 91.58 mmol, 4 eq.) was added slowly. The reaction mixture was stirred at 0° C. for 2 h. The reaction mixture was diluted with CH₂Cl₂ (100 mL), washed with water (100 mL×2), brine, dried over Na₂SO₄, and concentrated to provide the crude product, which was then purified on a silica gel column, eluting with ethyl acetate in pet ether (1:100-20:80) to obtained 3,3′-bis(bromomethyl)biphenyl as an off white solid (4.0 g, yield=51%), [C₁₄H₁₂Br₂]; ¹H NMR (500 MHz, CDCl₃) δ 7.62 (s, 2H), 7.53-7.50 (m, 2H), 7.44-7.37 (m, 4H), 4.56 (s, 4H).

### Step 4

### 4,4′-(([1,1′-biphenyl]-3,3′-diylbis(methylene))bis(oxy))bis(3-bromobenzaldehyde) (11)

To a stirred solution of 3,3′-bis(bromomethyl)biphenyl (1.5 g, 4.43 mmol) in ACN (30 mL) at room temperature were added 3-bromo-4-hydroxybenzaldehyde (2.6 g, 13.31 mmol) and K₂CO₃ (2.4 g, 17.751 mmol) and reaction mixture was stirred at 90° C. for 4 h. The reaction mixture was cooled to room temperature, diluted with cold water (50 mL) and extracted with EtOAc (2×50 mL). The combined organic layer was washed with brine (2×50 mL), dried over anhydrous Na₂SO₄, filtered and concentrated under reduced pressure to afford 4,4′-(([1,1′-biphenyl]-3,3′-diylbis(methylene))bis(oxy))bis(3-bromobenzaldehyde) (0.7 g, yield: 24%, LC-MS: 71%) as an off white solid. ¹H NMR (500 MHz, CDCl₃) δ 9.85 (s, 2H), 8.12 (m, 2H), 7.81-7.78 (m, 2H), 7.71 (s, 2H), 7.60-7.58 (m, 2H), 7.50-7.46 (m, 4H), 7.08 (d, J=11.0 Hz, 2H), 5.32 (s, 4H).

### Step 5

### (2S,2′S)-1,1′-(((([1,1′-biphenyl]-3,3′-diylbis(methylene))bis(oxy))bis(3-bromo-4, 1-phenylene))bis(methylene))bis(piperidine-2-carboxylic acid)

To a solution of 4,4′-(biphenyl-3,3′-diylbis(methylene))bis(oxy)bis(3-bromobenzaldehyde) (500 mg, 0.865 mmol) in MeOH (20 mL) were added (S)-piperidine-2-carboxylic acid (558.8 mg, 4.32 mmol) and AcOH (1 mL) and stirred at 80° C. for 4-5 h. The reaction mixture was cooled to room temperature, prior to the addition of NaCNBH₃ (270.3 mg, 4.125 mmol) and stirred at 80° C. for 3 h. The reaction mass was poured into ice-cold water, basified with sat. NaHCO₃ and solid thus precipitated was filtered and dried under vacuum to get crude compound. The crude product was purified by prep-HPLC (Mobile phase:0.1% TFA in H₂O: ACN; Column: SUNFIRE-C18 (150*30), 5u; Gradient: (T % B):—0/30, 11/44, 11.1/98, 13/98, 13.1/30, 13/30; Flow Rate: 25 ml/min; Diluent: ACN+H₂O+THF+1% of TFA) to get (2S,2′S)-1,1′-(((([1,1′-biphenyl]-3,3′-diylbis(methylene))bis(oxy))bis(3-bromo-4,1-phenylene))bis(methylene))bis(piperidine-2-carboxylic acid) (150 mg, yield: 21%, HPLC 99.02%) as an off white solid. ES+, m/z 804.8 [M+H]; [C₄₀H₄₂Br₂N₂O₆]; ¹H NMR (500 MHz, DMSO-d₆) δ 7.75 (s, 2H), 7.62-7.58 (m, 4H), 7.50 (t, J=7.5 Hz, 2H), 7.45 (d, J=7.5 Hz, 2H), 7.22 (dd, J=10.0 Hz, J=7.0 Hz, 2H), 7.10 (d, J=8.0 Hz, 2H), 5.27 (s, 4H), 3.80 (d, J=13.5 Hz, 2H), 3.16 (d, J=3.0 Hz, 2H), 2.79-2.77 (m, 2H), 2.63-2.52 (m, 2H), 1.88 (m, 2H), 1.66-1.63 (m, 6H), 1.59-1.57 (m, 4H), 1.54-1.53 (m, 2H). m.p.=306-310° C.

### HPLC Conditions:

Column: XBRIDE C18 (150 mm×4.6 mm, 3.5 μm)
Mobile phase: 10 mM Ammonium Acetate in H₂O, B: 100% Acetonitrile
Flow rate: 1.0 mL/min
T/% B (min): 0/5, 1.2/5, 3.0/55, 5.0/70, 7/95, 10/95, 12/100, 14.0/5, 16/5.

### Diluent: (ACN:H₂O)

### Temp: 25° C.

Ret. time: 5.22 min

### Purity: 99.02%

### Prep-HPLC Conditions:

### Column: SUNFIRE-C18 (150*30), 5u

Mobile phase: 0.1% TFA in H₂O:ACN
Flow rate: 25 mL/min
T/% B (min): 0/30, 11/44, 11.1/98, 13/98, 13.1/30, 13/30.

### Diluent: (ACN+H₂O+THF+1% of TFA)

### Temp: 25° C.

### Example 9

### (2S,2′S)-1,1′-(((((2,2′-Dichloro-[1,1′-biphenyl]-3,3′-diyl)bis(methylene))bis(oxy))bis(3-bromo-4,1-phenylene))bis(methylene))bis(piperidine-2-carboxylic acid) (Cpd 9)

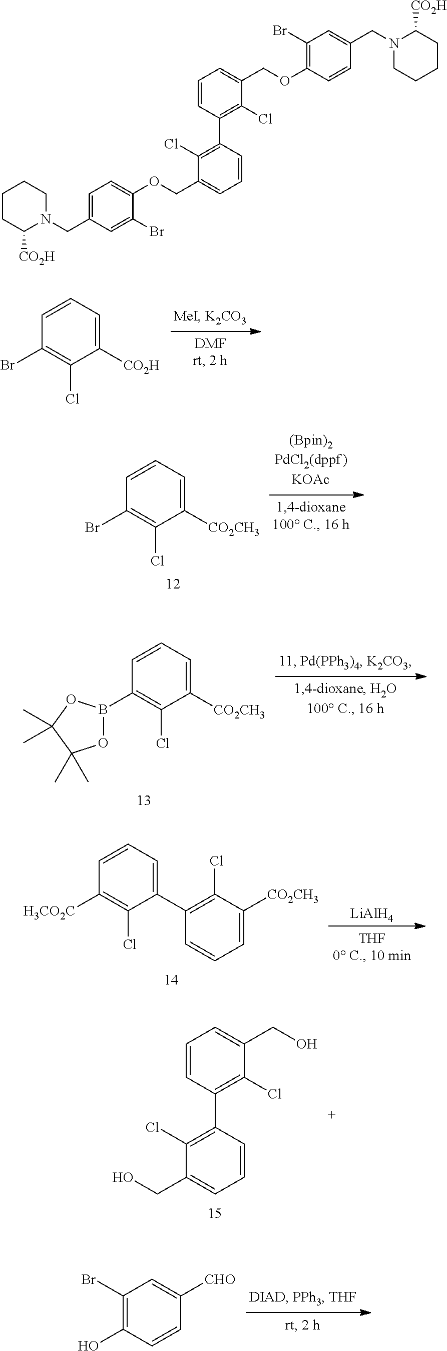

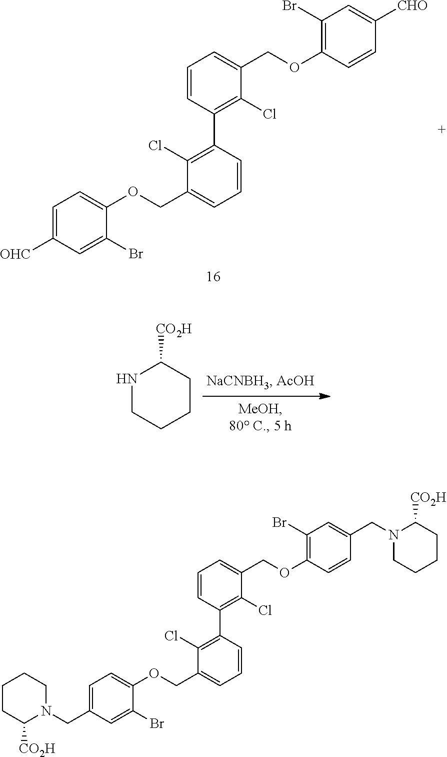 [SMILES: O=Cc1ccc(OCc2cccc(-c3cccc(COc4ccc(C=O)cc4Br)c3Cl)c2Cl)c(Br)c1 ; O=C(O)[C@@H]1CCCCN1 ; O=C(O)[C@@H]1CCCCN1Cc1ccc(OCc2cccc(-c3cccc(COc4ccc(CN5CCCC[C@H]5C(=O)O)cc4Br)c3Cl)c2Cl)c(Br)c1]

### Step 1

### Methyl 3-bromo-2-chlorobenzoate (12)

To a stirred solution of 3-bromo-2-chlorobenzoic acid (6 g, 25.64 mmol) in DMF (30 mL) at 0° C. were added K₂CO₃ (5.2 g, 38.46 mmol) and CH₃I (5.47 g, 38.46 mmol). The reaction mixture was allowed to warm to room temperature and stirred for 1 h. The reaction mixture was diluted with cold water and stirred for 10 minutes. The resulting precipitate was filtered and dried in vacuum to get methyl 3-bromo-2-chlorobenzoate (5.8 g, yield: 92%, LC-MS 99.5%). ¹H NMR (400 MHz, CDCl₃) δ 7.75 (dd, J=8.0 Hz, J=1.6 Hz, 1H), 7.68 (dd, J=7.6 Hz, J=1.6 Hz, 1H), 7.18 (t, J=7.8 Hz, 1H), 3.94 (s, 3H).

### Step 2

### Methyl 2-chloro-3-(4,4,5,5-tetramethyl-1,3,2-dioxaborolan-2-yl)benzoate (13)

To a stirred solution of methyl 3-bromo-2-chlorobenzoate (2.3 g, 9.27 mmol) in 1,4-dioxane (20 mL) at room temperature were added (Bpin)₂ (3.5 g, 13.91 mmol), KOAc (2.7 g, 27.8 mmol) and Pd₂(dppf).CH₂Cl₂ (0.34 g, 0.46 mmol) and degassed with argon for 5 min. The resulting mixture was maintained at 100° C. for 16 h. The reaction mixture was filtered through celite pad and the filtrate was concentrated under reduced pressure to get methyl 2-chloro-3-(4,4,5,5-tetramethyl-1,3,2-dioxaborolan-2-yl)benzoate (5 g crude compound) as a dark brown semi-solid. The crude product was used for the next step without purification.

### Step 3

### Dimethyl 2,2′-dichlorobiphenyl-3,3′-dicarboxylate (14)

To a stirred solution of methyl 3-bromo-2-chlorobenzoate (3 g, 12.09 mmol) in 1,4-dioxane at room temperature were added methyl 2-chloro-3-(4,4,5,5-tetramethyl-1,3,2-dioxaborolan-2-yl)benzoate (5.3 g, 18.14 mmol), K₂CO₃ (3.3 g, 24.18 mmol) and H₂O (5 mL) and degassed with argon for 5 min, prior to the addition of Pd(PPh₃)₄(0.837 g, 0.72 mmol). The resulting mixture was maintained at 100° C. for 16 h. The reaction mixture was diluted with water (50 mL), and extracted with ethyl acetate (3×35 mL). The combined organic layer was dried over Na₂SO₄ and concentrated under reduced pressure. The resulting crude product was purified on a silica gel column, eluting with ethyl acetate in pet-ether (5:95) to afford dimethyl 2,2′-dichlorobiphenyl-3,3′-dicarboxylate (2.8 g, yield: 70%, LC-MS 89.2%) as an off-white solid. ¹H NMR (500 MHz, CDCl₃) δ 7.75 (dd, J=7.2 Hz, J=2.4 Hz, 2H), 7.39-7.36 (m, 4H), 3.99 (s, 6H).

### Step 4

### (2,2′-Dichlorobiphenyl-3,3′-diyl)dimethanol (15)

To a stirred solution of dimethyl 2,2′-dichlorobiphenyl-3,3′-dicarboxylate (2.3 g, 6.8 mmol) in THF (25 mL) at 0° C., LiAlH₄ ((2 M in THF, 17 mL, 5 eq) was added drop wise and stirred at 0° C. for 10 min. The reaction mixture was quenched with saturated NH₄Cl solution (25 mL) at 0° C. and filtered through celite pad. The celite pad was washed with methanol: CH₂Cl₂ (15:85) (50 mL). The organic layer was separated and dried over Na₂SO₄ and concentrated under reduced pressure to get a brown oily residue, which upon trituration with n-pentane gave (2,2′-dichlorobiphenyl-3,3′-diyl)dimethanol (1.6 g, yield: 84.2%) as an off-white solid. ¹H NMR (500 MHz, DMSO-d₆) δ 7.57-7.55 (m, 2H), 7.36 (t, J=7.6 Hz, 2H), 7.21-7.19 (m, 2H), 4.86 (d, J=4.0 Hz, 4H), 2.05 (bs, 2H).

### Step 5

### 4,4′-(((2,2′-Dichloro-[1,1′-biphenyl]-3,3′-diyl)bis(methylene))bis(oxy))bis(3-bromobenzaldehyde) (16)

To a stirred solution of (2,2′-dichlorobiphenyl-3,3′-diyl)dimethanol (1.6 g, 5.67 mmol) and 3-bromo-4-hydroxybenzaldehyde (2.3 g, 11.34 mmol) in THF (15 mL) at 0° C. were added PPh₃ (3.7 g, 14.18 mmol) and DIAD (2.86 g, 14.18 mmol). The reaction mixture was allowed to attain room temperature and stirred for 2 h. The reaction mixture was diluted with ethyl acetate (50 mL) and washed with water (3×25 mL). The organic layer was dried over Na₂SO₄ and concentrated under reduced pressure to get crude compound, which was purified on a silica gel column, eluting with ethyl acetate in pet ether (20:80) to get 4,4′-(((2,2′-dichloro-[1,1′-biphenyl]-3,3′-diyl)bis(methylene))bis(oxy))bis(3-bromobenzaldehyde) (0.42 g, yield: 11.47%) as an off-white solid. [C₂₈H₁₈Br₂C₁₂O₄]; ¹H NMR (500 MHz, CDCl₃) δ 9.87 (s, 2H), 8.15 (d, J=2.0 Hz, 4H), 7.85-7.80 (m, 2H), 7.45 (t, J=7.6 Hz, 2H), 7.29 (dd, J=7.6 Hz, 1.2 Hz, 2H), 7.12 (d, J=8.8 Hz, 2H), 5.41 (s, 4H).

### Step 6

### (2S,2′S)-1,1′-(((((2,2′-dichloro-[1,1′-biphenyl]-3,3′-diyl)bis(methylene))bis(oxy))bis(3-bromo-4,1-phenylene))bis(methylene))bis(piperidine-2-carboxylic acid)

To a stirred solution of 4,4′-(2,2′-dichlorobiphenyl-3,3′-diyl)bis(methylene)bis(oxy)bis(3-bromobenzaldehyde) (0.42 g, 0.65 mmol) and L-pipecolic acid (0.42 g, 3.25 mmol) in methanol (5 mL) at room temperature acetic acid (0.2 mL) was added and the reaction mixture was heated at 80° C. for 2 h. The reaction mixture was allowed to cool to room temperature, prior to the addition of NaCNBH₃ (0.204 g, 3.2 mmol). The reaction mixture was again maintained at 80° C. for 3 h. The reaction mixture was concentrated to get the crude compound. The crude compound was purified by GRACE (normal phase) flash chromatography using methanol:CH₂Cl₂ (40:60) as eluent to afford (2S,2′S)-1,1′-(((((2,2′-dichloro-[1,1′-biphenyl]-3,3′-diyl)bis(methylene))bis(oxy))bis(3-bromo-4,1-phenylene))bis(methylene))bis(piperidine-2-carboxylic acid) (0.12 g, yield: 21.1%, LC-MS: 96.45%, HPLC: 96.53%) as an off-white solid. ES+, m/z 872.5 [M+H]; [C₄₀H₄₀Br₂N₂O₆]; ¹H NMR (500 MHz, DMSO-d₆) δ 7.75 (d, J=7.0 Hz, 2H), 7.59 (s, 2H), 7.52 (t, J=7.7 Hz, 2H), 7.37 (d, J=7.0 Hz, 2H), 7.29 (d, J=7.0 Hz, 2H), 7.18 (d, J=8.5 Hz, 2H), 5.29 (s, 4H), 3.81 (d, J=13.5 Hz, 2H), 3.55 (d, J=13.5 Hz, 2H), 2.94-2.92 (m, 2H), 2.89-2.81 (m, 2H) 2.12-2.08 (m, 2H), 1.80-1.71 (m, 2H), 1.70-1.60 (m, 2H), 1.58-1.40 (m, 6H), 1.32-1.28 (m, 2H). m.p.=184-188° C.

### HPLC Conditions:

Column: XBRIDE C18 (150 mm×4.6 mm, 3.5 μm)
Mobile phase: 10 mM AmmoniumAcetate in H₂O, B: 100% Acetonitrile
Flow rate: 1.0 mL/min
T/% B (min): 0/5, 1.2/5, 3.0/55, 5.0/70, 7/95, 10/95, 12/100, 14.0/5, 16/5.

### Diluent: (ACN:H₂O)

### Temp: 25° C.

Ret. time: 5.29 min

### Purity: 96.53%

### Example 10

### (2S,2′S)-1,1′-(((((2,2′-Difluoro-[1,1′-biphenyl]-3,3′-diyl)bis(methylene))bis(oxy))bis(3-bromo-4,1-phenylene))bis(methylene))bis(piperidine-2-carboxylic acid) (Cpd 10)

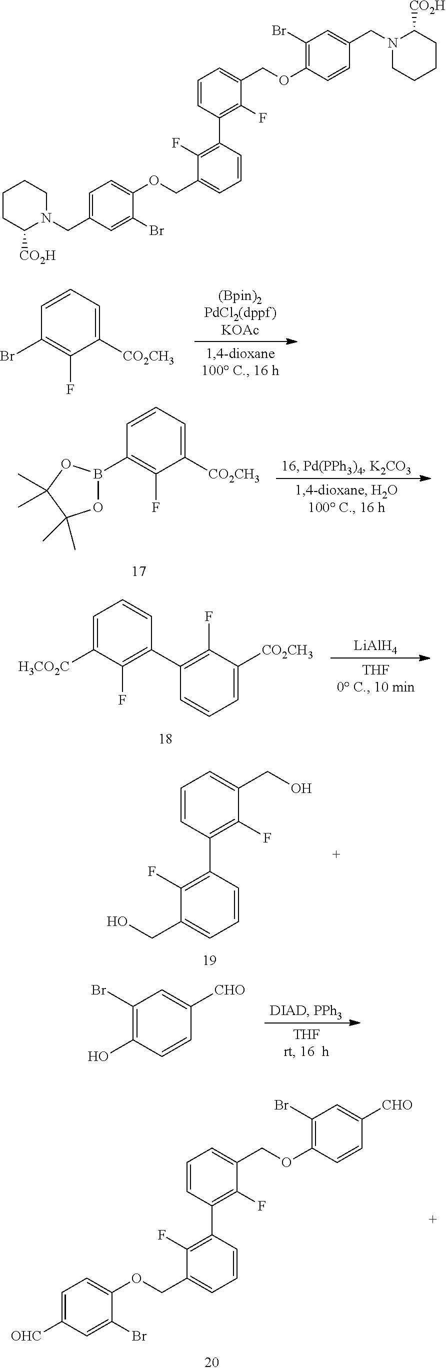

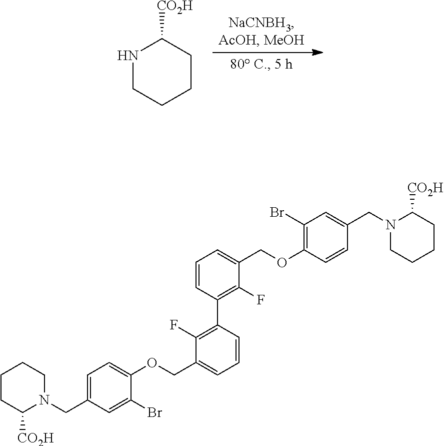 [SMILES: O=C(O)[C@@H]1CCCCN1 ; O=C(O)[C@@H]1CCCCN1Cc1ccc(OCc2cccc(-c3cccc(COc4ccc(CN5CCCC[C@H]5C(=O)O)cc4Br)c3F)c2F)c(Br)c1]

### Step 1

### Methyl 2-fluoro-3-(4,4,5,5-tetramethyl-1,3,2-dioxaborolan-2-yl)benzoate (17)

To a stirred solution of methyl 3-bromo-2-fluorobenzoate (5 g, 21.64 mmol) in 1,4-dioxane (50 mL) at room temperature were added (Bpin)₂ (8.2 g, 32.46 mmol), KOAc (6.37 g, 64.93 mmol) and PdCl₂(dppf) (1.7 g, 2.16 mmol) and degassed with argon for 5 min. The resulting mixture was then maintained at 100° C. for 16 h. The reaction mixture was filtered through celite pad and the filtrate was concentrated under reduced pressure to get methyl 2-fluoro-3-(4,4,5,5-tetramethyl-1,3,2-dioxaborolan-2-yl)benzoate (8 g, crude compound) as a dark brown solid. The crude product was used for the next step without purification.

### Step 2

### Dimethyl 2,2′-difluorobiphenyl-3,3′-dicarboxylate (18)

To a stirred solution of methyl 3-bromo-2-fluorobenzoate (4.5 g, 19.31 mmol) in 1,4-dioxane (40 mL) at room temperature were added methyl 2-fluoro-3-(4,4,5,5-tetramethyl-1,3,2-dioxaborolan-2-yl)benzoate (8 g, 28.96 mmol), K₂CO₃ (5 g, 38.6 mmol) and H₂O (3 mL) and degassed with argon for 5 min, prior to the addition of Pd(PPh₃)₄(1.3 g, 1.15 mmol). The resulting mixture was heated at 100° C. for 16 h. The reaction mixture was diluted with water (100 mL) and extracted with ethyl acetate (3×50 mL); the organic layer was dried over Na₂SO₄ and concentrated under reduced pressure. The resulting crude compound was purified on a silica gel column, eluting with ethyl acetate in pet ether (5:95) to get dimethyl 2,2′-difluorobiphenyl-3,3′-dicarboxylate (3.2 g, yield: 54%, LC/MS: 98%) as an off-white solid. ¹H NMR (500 MHz, CDCl₃) δ 8.01-7.98 (m, 2H), 7.57-7.53 (m, 2H), 7.29 (t, J=7.75 Hz, 2H), 3.94 (s, 6H).

### Step 3

### (2,2′-Difluorobiphenyl-3,3′-diyl)dimethanol (19)

To a stirred solution of dimethyl 2,2′-difluorobiphenyl-3,3′-dicarboxylate (3 g, 9.8 mmol) in THF (25 mL) at 0° C., LiAlH₄ ((2 M in THF, 24.5 mL, 5 eq) was added drop-wise and stirred at 0° C. for 10 min. The reaction mixture was quenched with saturated NH₄Cl solution (25 mL) at 0° C. and filtered through celite pad and the celite pad was washed with methanol: CH₂Cl₂ (15:85) (50 mL). The organic layer was separated, dried over Na₂SO₄ and concentrated under reduced pressure to get the crude compound as a brown oily liquid, which upon trituration with pentane afforded (2,2′-difluorobiphenyl-3,3′-diyl)dimethanol (2.4 g, yield: 96%) as an off-white solid. ¹H NMR (500 MHz, CDCl₃) δ 7.50-7.47 (m, 2H), 7.73-7.30 (m, 2H), 7.26-7.21 (m, 2H), 4.83 (s, 4H), 1.81 (bs, 2H).

### Step 4

### 4,4′-(((2,2′-difluoro-[1,1′-biphenyl]-3,3′-diyl)bis(methylene))bis(oxy))bis(3-bromobenzaldehyde) (20)

To a stirred solution of (2,2′-difluorobiphenyl-3,3′-diyl)dimethanol (2 g, 8.0 mmol) and 3-bromo-4-hydroxybenzaldehyde (3.21 g, 16.0 mmol) in THF (20 mL) at 0° C. were added PPh₃ (5.24 g, 20 mmol) and DIAD (4.04 g, 20 mmol). The reaction mixture was allowed to warm to room temperature and stirred for 2 h. The reaction mixture was diluted with ethyl acetate (50 mL) and washed with water (3×25 mL). The organic layer was dried over Na₂SO₄ and concentrated under reduced pressure to get brown solid. The crude product was purified on a silica gel column, eluting with ethyl acetate in pet-ether (20:80) to afford 4,4′-(((2,2′-difluoro-[1,1′-biphenyl]-3,3′-diyl)bis(methylene))bis(oxy))bis(3-bromobenzaldehyde) (0.6 g, yield: 12.24%, LC-MS: 91.16%) as an off white solid; [C₂₈H₁₈Br₂F₂O₄]; ¹H NMR (500 MHz, CDCl₃) δ 9.86 (s, 2H), 7.13 (d, J=2.5 Hz, 2H), 7.84 (dd, J=13.0 Hz, 8.0 Hz, 2H), 7.69 (m, 2H), 7.40 (m, 2H), 7.31 (t, J=9.5 Hz, 2H), 7.13 (d, J=10.5 Hz, 2H), 5.39 (s, 4H).

### Step 5

### (2S,2′S)-1,1′-(((((2,2′-difluoro-[1,1′-biphenyl]-3,3′-diyl)bis(methylene))bis(oxy))bis(3-bromo-4,1-phenylene))bis(methylene))bis(piperidine-2-carboxylic acid)

To a stirred solution of 4,4′-(2,2′-difluorobiphenyl-3,3′-diyl)bis(methylene)bis(oxy)bis(3-bromobenzaldehyde) (0.4 g, 0.65 mmol) and L-pipecolic acid (0.421 g, 3.25 mmol) in methanol (10 mL) at room temperature, catalytic amount of acetic acid (0.2 mL) was added. The reaction mixture was heated at 80° C. for 2 h. The reaction mixture was allowed to cool to room temperature, prior to the addition of NaCNBH₃ (0.205 g, 3.25 mmol). The reaction mixture was again heated at 80° C. for 3 h. The reaction mixture was concentrated and purified by GRACE flash chromatography (40% MeOH—CH₂Cl₂, silica gel) to afford (2S,2′S)-1,1′-(((((2,2′-difluoro-[1,1′-biphenyl]-3,3′-diyl)bis(methylene))bis(oxy))bis(3-bromo-4,1-phenylene))bis(methylene))bis(piperidine-2-carboxylic acid) (38 mg, yield: 6.9%, HPLC: 98.83%) as an off white solid. ES+, m/z 838.5 [M+H]; [C₄₀H₄₀F₂Br₂N₂O₆]; ¹H NMR (500 MHz, DMSO-d₆) δ 7.67 (bs, 2H), 7.58 (s, 2H), 7.47 (bs, 2H), 7.38 (t, J=7.75 Hz, 2H), 7.26 (d, J=8.0 Hz, 2H), 7.71 (d, J=8.51 Hz, 2H), 5.27 (s, 4H), 3.81 (d, J=13.0 Hz, 2H), 3.23 (d, J=13.0 Hz, 2H), 2.79 (d, J=5.5 Hz, 4H), 1.94 (s, 2H) 1.68-1.52 (m, 6H), 1.38-1.36 (m, 4H), 1.28 (bs, 2H). m.p.=204-208° C.

### HPLC Conditions:

Column: XBRIDE C18 (150 mm×4.6 mm, 3.5 μm)
Mobile phase: 10 mM AmmoniumAcetate in H₂O, B: 100% Acetonitrile
Flow rate: 1.0 mL/min
T/% B (min): 0/5, 1.2/5, 3.0/55, 5.0/70, 7/95, 10/95, 12/100, 14.0/5, 16/5.

### Diluent: (ACN:H₂O)

### Temp: 25° C.

Ret. time: 5.19 min

### Purity: 98.83%

### Example 11

### (2S,2′S)-1,1′-(((((2,2′-Dimethyl-[1,1′-biphenyl]-3,3′-diyl)bis(methylene))bis(oxy))bis(3-methyl-4,1-phenylene))bis(methylene))bis(piperidine-2-carboxylic acid) (Cpd 11)

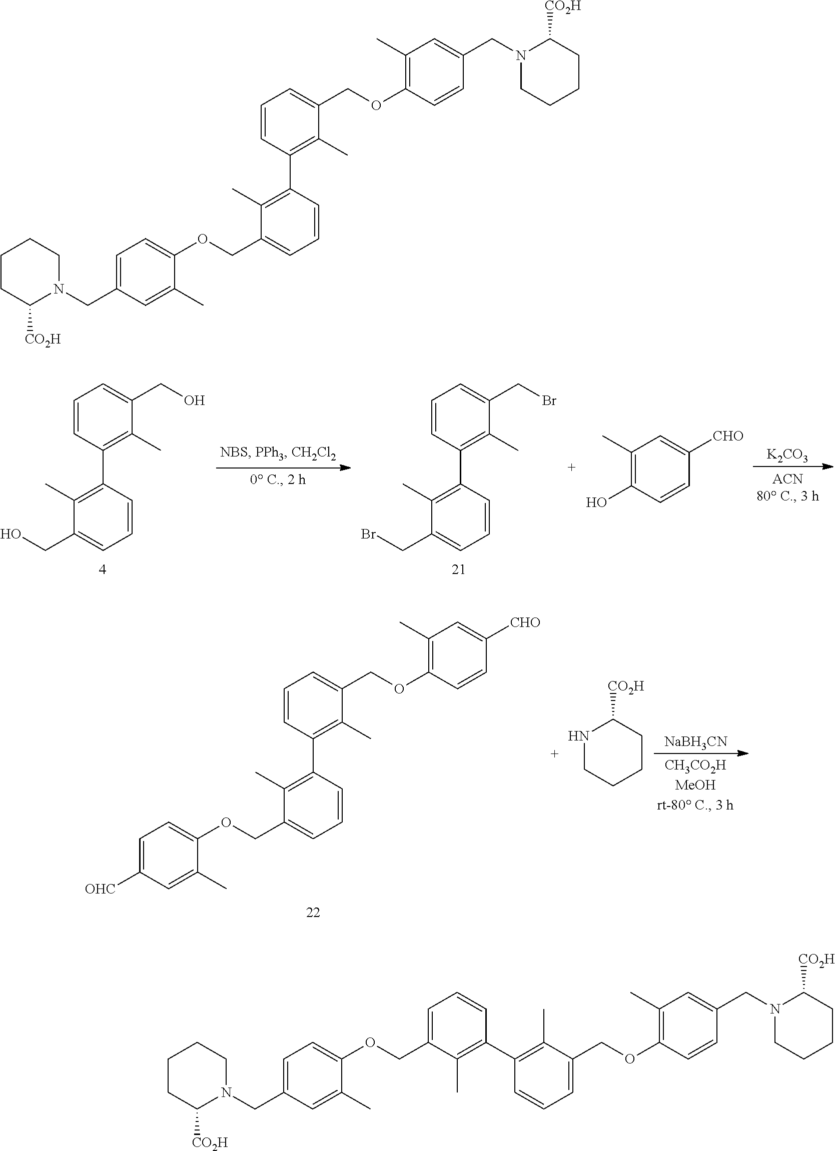

### Step 1

### 3,3′-Bis(bromomethyl)-2,2′-dimethylbiphenyl (21)

To a solution of (2,2′-dimethylbiphenyl-3,3′-diyl)dimethanol (10 g, 41.32 mmol) and Ph₃P (33 g, 123.96 mmol, 3 eq.) in CH₂Cl₂ (100 mL) at 0° C., NBS (165.28 g, 30 mmol, 4 eq.) was added slowly. The reaction mixture was stirred at 0° C. for 2 h. The reaction mixture was diluted with CH₂Cl₂ (100 mL), washed with water (2×100 mL), brine, dried over Na₂SO₄, and concentrated to provide the crude product, which was then purified on a silica gel column, eluting with ethyl acetate in pet ether (1:100-20:80) to obtained 3,3′-bis(bromomethyl)-2,2′-dimethylbiphenyl as a white solid (10 g, yield=66%), [C₁₆H₁₆ Br₂]; ¹H NMR (500 MHz, CDCl₃) δ 7.35-7.33 (m, 2H), 7.24-7.19 (m, 2H), 7.09-7.07 (m, 2H), 4.58 (s, 4H), 2.10 (s, 6H).

### Step 2

### 4,4′-(((2,2′-dimethyl-[1,1′-biphenyl]-3,3′-diyl)bis(methylene))bis(oxy))bis(3-methylbenzaldehyde) (22)

To a stirred solution of 3,3′-bis(bromomethyl)-2,2′-dimethylbiphenyl (1 g, 2.732 mmol) in ACN (20 mL) at room temperature were added K₂CO₃ (1.5 g, 10.928 mmol) and 4-hydroxy-3-methylbenzaldehyde (1.2 g, 8.196 mmol) and stirred at 80° C. for 3 h. The reaction mixture was concentrated to remove most of the solvent. The residue was poured into ice-water and the resulting precipitate was filtered and dried in vacuum to get 4,4′-(((2,2′-dimethyl-[1,1′-biphenyl]-3,3′-diyl)bis(methylene))bis(oxy))bis(3-methylbenzaldehyde) (1.1 g, yield: 67%, LC/MS 95%); ¹H NMR (500 MHz, CDCl₃) δ 9.88 (s, 2H), 7.74-7.73 (m, 4H), 7.48 (d, J=7.5 Hz, 2H), 7.30 (d, J=7.5 Hz, 2H), 7.18 (d, J=6.5 Hz, 2H), 7.06 (d, J=8.5 Hz, 2H), 5.20 (s, 4H), 2.32 (s, 6H), 2.07 (s, 6H).

### Step 3

### (2S,2′S)-1,1′-(((((2,2′-dimethyl-[1,1′-biphenyl]-3,3′-diyl)bis(methylene))bis(oxy))bis(3-methyl-4,1-phenylene))bis(methylene))bis(piperidine-2-carboxylic acid)

To a solution of 4,4′-(((2,2′-dimethyl-[1,1′-biphenyl]-3,3′-diyl)bis(methylene))bis(oxy))bis(3-methylbenzaldehyde) (700 mg, 1.67 mmol) in MeOH (20 mL) were added L-pipecolic acid (930 mg, 8.364 mmol) and acetic acid (3-4 drops) and the reaction mixture was stirred at 80° C. for 4-5 h. The reaction mixture was cooled to room temperature, prior to the addition of NaCNBH₃ (450 mg, 8.3647 mmol). The reaction mixture was stirred at 80° C. for 3 h. The reaction mixture was poured into ice cold water, and solid thus precipitated was filtered and dried under vacuum. This crude compound was purified by GRACE flash chromatography (40% MeOH—CH₂Cl₂, silica gel) to afford (2S,2′S)-1,1′-(((((2,2′-dimethyl-[1,1′-biphenyl]-3,3′-diyl)bis(methylene))bis(oxy))bis(3-methyl-4,1-phenylene))bis(methylene))bis(piperidine-2-carboxylic acid) (110 mg, yield: 12%, HPLC 98.11%) as an off white solid. ES−, m/z 703.1 [M−H]; [C₄₄H₅₂N₂O₆]; ¹H NMR (500 MHz, DMSO-d₆) δ 7.48 (d, J=7.5 Hz, 2H), 7.28 (t, J=6.0 Hz, 2H), 7.13-7.08 (m, 6H), 7.00 (d, J=8.0 Hz, 2H), 5.12 (s, 4H), 3.85 (dd, J=15.0 Hz, J=10.5 Hz, 2H), 3.30 (d, J=12.5 Hz, 2H), 2.86-2.79 (m, 4H), 2.15 (s, 6H), 2.03-1.97 (m, 8H), 1.71-1.70 (m, 2H), 1.63-1.62 (m, 2H), 1.51-1.46 (m, 6H), 1.23-1.22 (m, 2H). m.p.=191-195° C.

### HPLC Conditions:

Column: XBRIDE C18 (150 mm×4.6 mm, 3.5 μm)
Mobile phase: 10 mM AmmoniumAcetate in H₂O, B: 100% Acetonitrile
Flow rate: 1.0 mL/min
T/% B (min): 0/5, 1.2/5, 3.0/55, 5.0/70, 7/95, 10/95, 12/100, 14.0/5, 16/5.

### Diluent: (ACN:H₂O)

### Temp: 25° C.

Ret. time: 5.29 min

### Purity: 98.1%

### Example 12

### (2S,2′S)-1,1′-(((((2,2′-Dimethyl-[1,1′-biphenyl]-3,3′-diyl)bis(methylene))bis(oxy))bis(3-chloro-4,1-phenylene))bis(methylene))bis(piperidine-2-carboxylic acid) (Cpd 12)

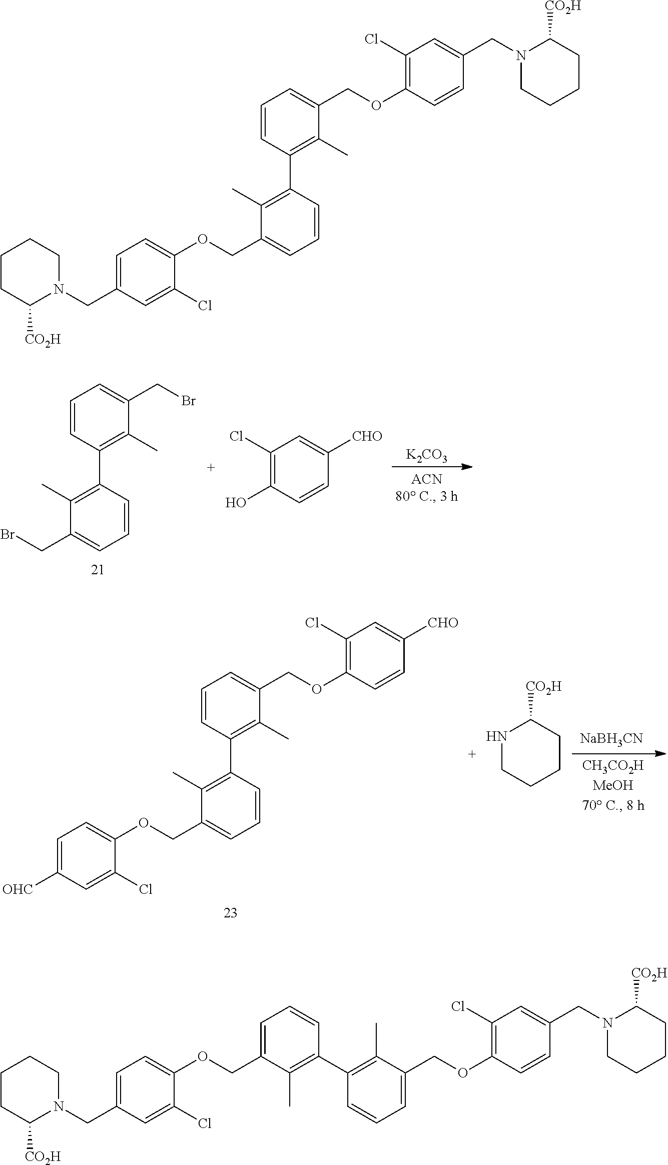

### Step 1

### 4,4′-(2,2′-dimethylbiphenyl-3,3′-diyl)bis(methylene)bis(oxy)bis(3-chlorobenzaldehyde) (23)

To a stirred solution of 3,3′-bis(bromomethyl)-2,2′-dimethylbiphenyl (1.1 g, 3.00 mmol) in ACN (20 mL) at room temperature were added K₂CO₃ (1.6 g, 12.02 mmol) and 3-chloro-4-hydroxybenzaldehyde (1.4 g, 9.01 mmol) and stirred at 80° C. for 3 h. The solvent was removed by rotavaporation. The residue was poured into ice-water and the resulting precipitate was filtered and dried in vacuum to get 4,4′-(((2,2′-dimethyl-[1,1′-biphenyl]-3,3′-diyl)bis(methylene))bis(oxy))bis(3-chlorobenzaldehyde) (1.1 g, yield: 70%, LC-MS 87%), [C₃₀H₂₄C₁₂O₄]; ¹H NMR (500 MHz, CDCl₃) δ 9.87 (s, 2H), 7.95-7.94 (m, 2H), 7.79 (t, J=2.0 Hz, 2H), 7.51 (d, J=7.0 Hz, 2H), 7.30 (t, J=7.5 Hz, 2H), 7.18-7.16 (m, 4H), 5.27 (s, 4H), 2.08 (s, 6H).

### Step 2

### (2S,2′S)-1,1′-(((((2,2′-dimethyl-[1,1′-biphenyl]-3,3′-diyl)bis(methylene))bis(oxy))bis(3-chloro-4,1-phenylene))bis(methylene))bis(piperidine-2-carboxylic acid)

To a solution of 4,4′-(((2,2′-dimethyl-[1,1′-biphenyl]-3,3′-diyl)bis(methylene))bis(oxy))bis(3-chlorobenzaldehyde) (500 mg, 0.965 mmol) in MeOH (20 mL) were added (S)-piperidine-2-carboxylic acid (625 mg, 4.853 mmol) and acetic acid (1 mL) and the reaction mixture was stirred at 80° C. for 4-5 h. The reaction mixture was cooled to room temperature, prior to the addition of NaCNBH₃ (325 mg, 4.852 mmol) and then stirred at 80° C. for 3 h. The reaction mixture was poured into ice-cold water, and solid thus precipitated was filtered and dried under vacuum. The resulting crude compound was purified by GRACE flash chromatography (40% MeOH in CH₂C₁₂, silica gel) to afford (2S,2′S)-1,1′-(((((2,2′-dimethyl-[1,1′-biphenyl]-3,3′-diyl)bis(methylene))bis(oxy))bis(3-chloro-4,1-phenylene))bis(methylene))bis(piperidine-2-carboxylic acid) (250 mg, yield: 35%, HPLC 98.95%) as an off-white solid. ES−, m/z 743.0 [M−H]; [C₄₂H₄₆C₁₂N₂O₆]; ¹H NMR (500 MHz, DMSO-d₆) δ 7.51 (d, J=7.0 Hz, 2H), 7.45 (s, 2H), 7.29 (d, J=7.5 Hz, 2H), 7.23-7.18 (m, 4H), 7.10 (d, J=7.5 Hz, 2H), 5.21 (s, 4H), 3.84 (d, J=13.5 Hz, 2H), 3.15 (d, J=12.5 Hz, 2H), 2.78 (d, J=6.0 Hz, 2H), 2.57 (d, J=6.0 Hz, 2H), 2.00 (s, 6H), 1.84 (t, J=9.0 Hz, 2H), 1.66-1.58 (m, 6H), 1.56-1.54 (m, 4H), 1.38-1.37 (m, 2H). m.p.=184-188° C.

### HPLC Conditions:

Column: XBRIDE C18 (150 mm×4.6 mm, 3.5 μm)
Mobile phase: 10 mM AmmoniumAcetate in H₂O, B: 100% Acetonitrile
Flow rate: 1.0 mL/min
T/% B (min): 0/5, 1.2/5, 3.0/55, 5.0/70, 7/95, 10/95, 12/100, 14.0/5, 16/5.

### Diluent: (ACN:H₂O)

### Temp: 25° C.

Ret. time: 5.25 min

### Purity: 98.95%

### Example 13

### (2S,2′S)-1,1′-(((((2,2′-Dimethyl-[1,1′-biphenyl]-3,3′-diyl)bis(methylene))bis(oxy))bis(3-(trifluoromethyl)-4,1-phenylene))bis(methylene))bis(piperidine-2-carboxylic acid) (Cpd 13)

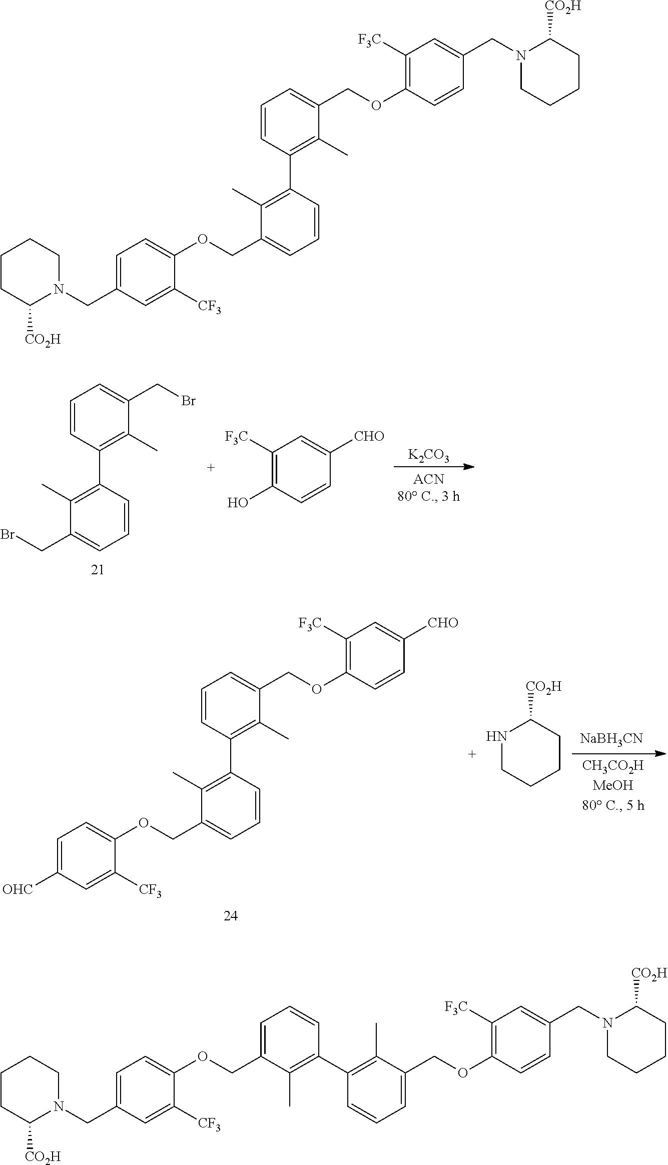

### Step 1

### 4,4′-(((2,2′-dimethyl-[1,1′-biphenyl]-3,3′-diyl)bis(methylene))bis(oxy))bis(3-(trifluoromethyl)benzaldehyde) (24)

To a stirred solution of 3,3′-bis(bromomethyl)-2,2′-dimethylbiphenyl (650 g, 1.7486 mmol) in ACN (25 mL) at room temperature were added K₂CO₃ (960 g, 6.99 mmol) and 4-hydroxy-3-(trifluoromethyl)benzaldehyde (1 g, 5.245 mmol) and stirred at 80° C. for 3 h. The reaction mixture was concentrated to remove most of the solvent. The residue was poured into ice-water and the resulting precipitate was filtered and dried in vacuum to get 4,4′-(((2,2′-dimethyl-[1,1′-biphenyl]-3,3′-diyl)bis(methylene))bis(oxy))bis(3-(trifluoromethyl)benzaldehyde) (470 g, yield: 47%, LC/MS 96%), [C₃₂H₂₄F₆O₄]; ¹H NMR (500 MHz, CDCl₃) δ 9.94 (s, 2H), 8.16 (s, 2H), 8.15 (d, J=1.5 Hz, 2H), 7.50 (dd, J=10.5 Hz, 6.5 Hz, 2H),7.48 (d, J=7.5 Hz, 2H), 7.31-7.28 (m, 4H), 5.31 (q, J=10.5 Hz, 4H), 2.04 (s, 6H).

### Step 2

### (2S,2′S)-1,1′-(((((2,2′-dimethyl-[1,1′-biphenyl]-3,3′-diyl)bis(methylene))bis(oxy))bis(3-(trifluoromethyl)-4,1-phenylene))bis(methylene))bis(piperidine-2-carboxylic acid)

To a solution of 4,4′-(((2,2′-dimethyl-[1,1′-biphenyl]-3,3′-diyl)bis(methylene))bis(oxy))bis(3-(trifluoromethyl)benzaldehyde) 5, (470 mg, 0.802 mmol) in MeOH (20 mL) were added L-pipecolic acid (518 mg, 4.012 mmol) and acetic acid (1 mL) and the reaction mixture was stirred at 80° C. for 4-5 h. The reaction mixture was cooled to room temperature, prior to the addition of NaCNBH₃ (250 mg, 4.01 mmol) and then stirred at 80° C. for 3 h. The reaction mixture was poured into ice cold water, and solid thus precipitated was filtered and dried under vacuum. The crude compound was purified by GRACE flash chromatography (40% MeOH in CH₂Cl₂, silica gel) to obtain (2S,2′S)-1,1′-(((((2,2′-dimethyl-[1,1′-biphenyl]-3,3′-diyl)bis(methylene))bis(oxy))bis(3-(trifluoromethyl)-4,1-phenylene))bis(methylene))bis(piperidine-2-carboxylic acid) (150 mg, yield: 23%, HPLC 95.29%) as an off white solid. ES−, m/z 811 [M−H]; [C₄₄H₄₆F₆N₂O₆]; ¹H NMR (500 MHz, DMSO-d₆) δ 7.62 (s, 2H), 7.57 (d, J=8.5 Hz, 2H), 7.50 (d, J=7.5 Hz, 2H), 7.34 (d, J=8.5 Hz, 2H), 7.29 (t, J=7.5 Hz, 2H), 7.09 (d, J=7.5 Hz, 2H), 5.27 (q, J=12.0 Hz, 4H), 3.92 (d, J=13.5 Hz, 2H), 3.21 (d, J=13.5 Hz, 2H), 2.78-2.75 (m, 2H), 2.58-2.57 (m, 2H), 1.98 (s, 6H), 1.85 (t, J=9.0 Hz, 2H), 1.65-1.53 (m, 6H), 1.39-1.31 (m, 4H), 1.23-1.22 (m, 2H). m.p.=287-291° C.

### HPLC Conditions:

Column: XBRIDE C18 (150 mm×4.6 mm, 3.5 μm)
Mobile phase: 10 mM AmmoniumAcetate in H₂O, B: 100% Acetonitrile
Flow rate: 1.0 mL/min
T/% B (min): 0/5, 1.2/5, 3.0/55, 5.0/70, 7/95, 10/95, 12/100, 14.0/5, 16/5.

### Diluent: (ACN:H₂O)

### Temp: 25° C.

Ret. time: 5.54 min

### Purity: 95.29%

### Example 14

### 2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-methylpropanoic acid) di-trifluoroacetic acid salt (Cpd 14)

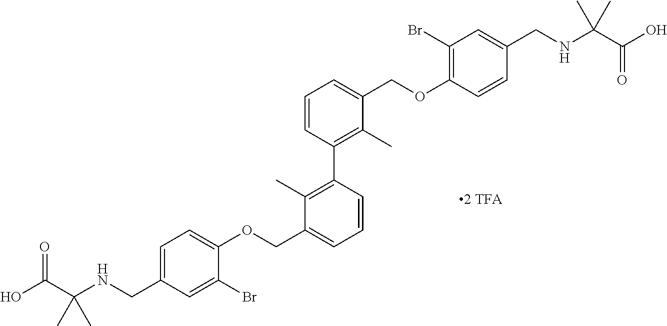 [SMILES: Cc1c(COc2ccc(CNC(C)(C)C(=O)O)cc2Br)cccc1-c1cccc(COc2ccc(CNC(C)(C)C(=O)O)cc2Br)c1C]

To a solution of 4,4′-(((2,2′-dimethyl-[1,1′-biphenyl]-3,3′-diyl)bis(methylene))bis(oxy))bis(3-bromobenzaldehyde) 5 (500 mg, 0.825 mmol) in MeOH (25 mL) were added 2-amino-2-methylpropanoic acid (0.425 mg, 4.125 mmol) and acetic acid (3-4 drops) and the reaction mixture was stirred at 80° C. for 4-5 h; the reaction mixture was cooled to room temperature, prior to the addition of NaCNBH₃ (259.2 mg, 4.125 mmol). The reaction mixture was stirred at 80° C. for 3 h. The reaction mixture was poured into ice cold water, basified with saturated NaHCO₃ and solid thus precipitated was filtered and dried under vacuum to get crude compound. The crude neutral product was purified by prep-HPLC under acidic conditions (Mobile phase: 0.1% TFA in H₂O:ACN; Column: YMC-TRIART C18 150×19 mm 5 um; Gradient: (T % B): 0/40, 8/85, 10/85, 10.1/98, 12/98, 12.1/40, 14/40; Flow Rate: 25 ml/min; Diluent: ACN+H₂O+THF+1% of TFA) to give 2,2′-((((((2,2′-dimethyl-[1,1′-biphenyl]-3,3′-diyl)bis(methylene))bis(oxy))bis(3-bromo-4,1-phenylene))bis(methylene))bis(azanediyl))bis(2-methylpropanoic acid) (200 mg, as the di-TFA salt, yield: 34%, UPLC 95.6%) as a white solid. ES−, m/z 779.1 [M−H]; [C₃₈H₄₂Br₂N₂O₆] ¹H NMR (500 MHz, DMSO-d₆) δ 7.75 (d, J=2.0 Hz, 2H), 7.54 (d, J=7.0 Hz, 2H), 7.44 (d, J=2.0 Hz, 2H), 7.35 (d, J=8.5 Hz, 2H), 7.30 (t, J=6.8 Hz, 2H), 7.10 (d, J=6.5 Hz, 2H), 5.29 (s, 4H), 4.01 (s, 4H), 2.01 (s, 6H), 1.46 (s, 12H). m.p.=230-234° C.

### Example 15

### (2S,2′S)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))dipropionic acid di-trifluoroacetic acid salt (Cpd 15)

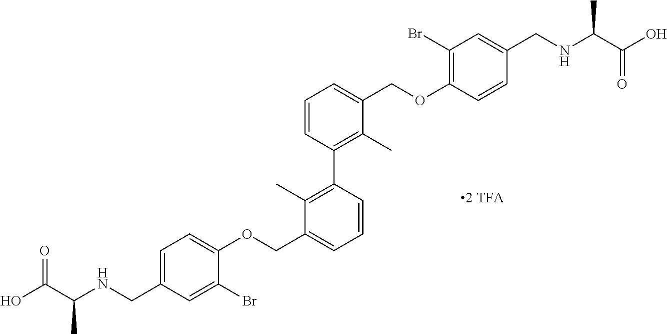 [SMILES: Cc1c(COc2ccc(CN[C@@H](C)C(=O)O)cc2Br)cccc1-c1cccc(COc2ccc(CN[C@@H](C)C(=O)O)cc2Br)c1C]

To a solution of 4,4′-(((2,2′-dimethyl-[1,1′-biphenyl]-3,3′-diyl)bis(methylene))bis(oxy))bis(3-bromobenzaldehyde) 5 (500 mg, 0.82 mmol) in MeOH (20 mL) were added (S)-2-aminopropanoic acid (367.1 mg, 4.12 mmol) and AcOH (3-4 drops) and stirred at 80° C. for 4-5 h. The reaction mixture was cooled to room temperature, prior to the addition of NaCNBH₃ (259.2 mg, 4.125 mmol). The reaction mixture was stirred at 80° C. for 3 h. The reaction mixture was poured into ice cold water, basified with sat. NaHCO₃ and solid thus precipitated was filtered and dried under vacuum to get crude compound. The crude neutral product was purified by prep-HPLC twice under acidic conditions (Mobile phase: 0.1% TFA in H₂O:ACN; Column: INERTSIL-ODS (250*20), 5u; Gradient (% B): 0/20, 1/20, 8/55, 10/55, 10.1/98, 11/98, 11.1/20, 14/20; Flow Rate: 20 mL/min; Diluent: ACN+THF+H₂O+1% TFA) to get (2S,2′S)-2,2′-((((((2,2′-dimethyl-[1,1′-biphenyl]-3,3′-diyl)bis(methylene))bis(oxy))bis(3-bromo-4,1-phenylene))bis(methylene))bis(azanediyl))dipropionic acid (80 mg, as the di-TFA salt, yield: 13%, HPLC 97.08%) as an off-white solid. ES−, m/z 750.8 [M−H]; [C₃₆H₃₈Br₂N₂O₆]; ¹H NMR (500 MHz, DMSO-d₆) δ 7.75 (d, J=1.5 Hz, 2H), 7.54 (d, J=7.0 Hz, 2H), 7.46-7.29 (m, 2H), 7.35 (d, J=8.5 Hz, 2H), 7.30 (t, 2H), 7.11 (d, J=7.5 Hz, 2H), 5.28 (s, 4H), 4.04 (s, 4H), 3.72-3.71 (m, 2H), 2.01 (s, 6H) 1.41 (d, J=7.0 Hz, 6H). m.p.=243-247° C.

### Example 16

### (2R,2′R)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))dipropionic acid di-trifluoroacetic acid salt (Cpd 16)

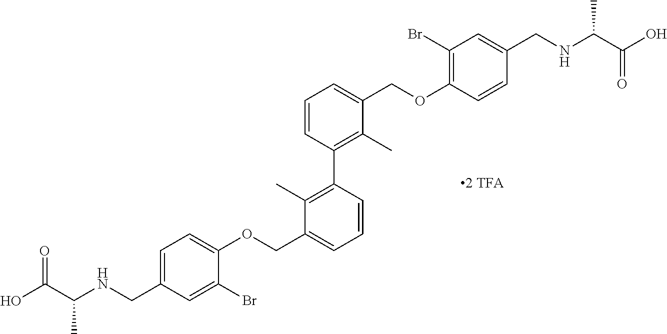 [SMILES: Cc1c(COc2ccc(CN[C@H](C)C(=O)O)cc2Br)cccc1-c1cccc(COc2ccc(CN[C@H](C)C(=O)O)cc2Br)c1C]

To a solution of 4,4′-(((2,2′-dimethyl-[1,1′-biphenyl]-3,3′-diyl)bis(methylene))bis(oxy))bis(3-bromobenzaldehyde) 5 (500 mg, 0.82 mmol) in MeOH (20 mL) were added (R)-2-aminopropanoic acid (367.1 mg, 4.12 mmol) and AcOH (3-4 drops) and stirred at 80° C. for 4-5 h; The reaction mixture was cooled to room temperature, prior to the addition of NaCNBH₃ (259.2 mg, 4.125 mmol). The reaction mixture was stirred at 80° C. for 3 h and poured into ice-cold water, basified with sat NaHCO₃ and solid thus precipitated was filtered and dried under vacuum to get crude compound. The crude neutral product was purified by prep-HPLC twice under acidic conditions (Mobile phase: 0.1% TFA in H₂O: ACN; Column: YMC-TRIART-C18-(150*25 MM), 10u; Gradient (% B): 0/20, 8/60, 9/60, 9.1/98, 12/98, 12.1/20, 14/20; Flow Rate: 20 mL/min; Diluent: ACN+THF+H₂O+1% TFA) to get (2R,2′R)-2,2′-((((((2,2′-dimethyl-[1,1′-biphenyl]-3,3′-diyl)bis(methylene))bis(oxy))bis(3-bromo-4,1-phenylene))bis(methylene))bis(azanediyl))dipropionic acid (150 mg, as the di-TFA salt, yield: 24%, HPLC 97.45%) as a white solid. ES−, m/z 750.8 [M−H]; [C₃₆H₃₈Br₂N₂O₆]; ¹H NMR (500 MHz, DMSO-d₆) δ 14.0 (s, 1H), 9.23-9.21 (m, 3H), 7.77 (d, J=2.0 Hz, 2H), 7.55 (d, J=7.0 Hz, 2H), 7.49-7.47 (m, 2H), 7.39 (d, J=8.5 Hz, 2H), 7.32 (d, J=7.5 Hz, 2H), 7.11 (d, J=7.0 Hz, 2H), 5.29 (s, 4H), 4.13 (m, 4H), 3.99 (q, J=7.0 Hz, 2H), 2.03 (s, 6H) 1.48 (d, J=7.5 Hz, 6H). m.p.=186-190° C.

### Example 17

### (2R,2′R)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-hydroxypropanoic acid) di-trifluoroacetic acid salt (Cpd 17)

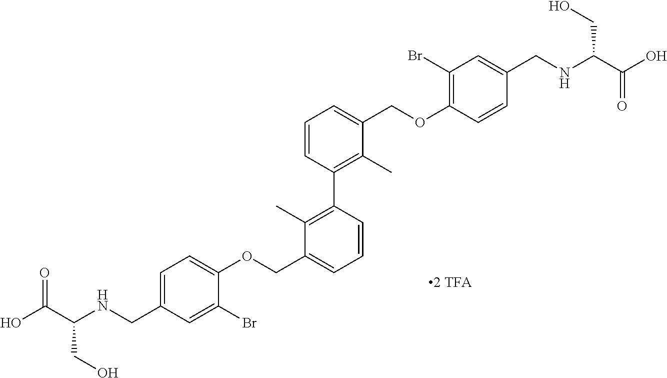 [SMILES: Cc1c(COc2ccc(CN[C@H](CO)C(=O)O)cc2Br)cccc1-c1cccc(COc2ccc(CN[C@H](CO)C(=O)O)cc2Br)c1C]

To a solution of 4,4′-(((2,2′-dimethyl-[1,1′-biphenyl]-3,3′-diyl)bis(methylene))bis(oxy))bis(3-bromobenzaldehyde) 5 (400 mg, 0.66 mmol) in MeOH (20 mL) were added (R)-2-amino-3-hydroxypropanoic acid (346.8 mg, 3.30 mmol) and AcOH (3-4 drops) and stirred at 80° C. for 4 h; The reaction mixture was cooled to room temperature, prior to the addition of NaCNBH₃ (207.3 mg, 3.30 mmol). The reaction mixture was stirred at 80° C. for 3 h. The reaction mixture was poured into ice cold water and solid thus precipitated was filtered and dried under vacuum to get crude compound. The crude neutral product was purified by prep-HPLC twice under acidic conditions (Mobile phase: 0.1% TFA in H₂O:ACN; Column: X-BRIDGE-C18 (150*19), 5u; Gradient (% B): 0/10, 1/10, 8/60, 8.1/98, 9/98, 9.1/10, 11/10; Flow Rate: 20 mL/min; Diluent: ACN+MeOH+H₂O+1% TFA) to give (2R,2′R)-2,2′-((((((2,2′-dimethyl-[1,1′-biphenyl]-3,3′-diyl)bis(methylene))bis(oxy))bis(3-bromo-4,1-phenylene))bis(methylene))bis(azanediyl))bis(3-hydroxypropanoic acid) (75 mg, as the di-TFA salt, yield: 13%, HPLC 95.19%) as a white solid. ES+, m/z 785.0 [M+H]; [C₃₆H₃₈Br₂N₂O₈] ¹H NMR (500 MHz, DMSO-d₆) δ 7.74 (d, J=2.0 Hz, 2H), 7.54 (d, J=7.5 Hz, 2H), 7.43 (dd, J=10.5 Hz, J=6.5 Hz, 2H), 7.30 (t, J=8.5 Hz, 4H), 7.11 (d, J=7.0 Hz, 2H), 5.26 (s, 4H), 4.01 (m, 4H), 3.77-3.73 (m, 4H), 3.47 (s, 2H), 2.02 (s, 6H). m.p.=156-160° C.

### Example 18

### (2R,2′R,3S,3′S)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-hydroxybutanoic acid) di-trifluoroacetic acid salt (Cpd 18)

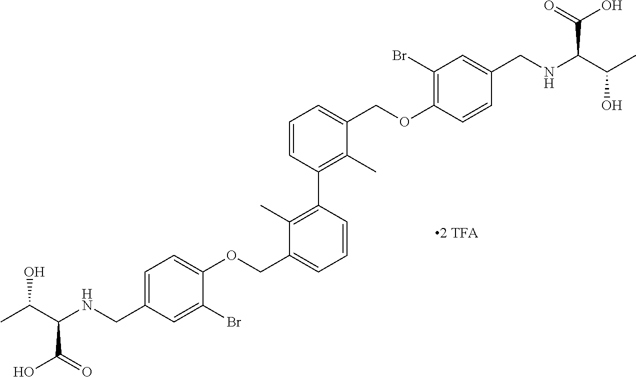 [SMILES: Cc1c(COc2ccc(CN[C@@H](C(=O)O)[C@H](C)O)cc2Br)cccc1-c1cccc(COc2ccc(CN[C@@H](C(=O)O)[C@H](C)O)cc2Br)c1C]

To a solution of 4,4′-(((2,2′-dimethyl-[1,1′-biphenyl]-3,3′-diyl)bis(methylene))bis(oxy))bis(3-bromobenzaldehyde) 5 (500 mg, 0.82 mmol) in MeOH (25 mL) were added (2R,3S)-2-amino-3-hydroxybutanoic acid (491 mg, 4.12 mmol) and AcOH (3-4 drops) and stirred at 80° C. for 4-5 h. The reaction mixture was cooled to room temperature, prior to the addition of NaCNBH₃ (259.2 mg, 4.125 mmol). The reaction mixture was stirred at 80° C. for 4 h. The reaction mixture was poured into ice cold water and solid thus precipitated was filtered and dried under vacuum to get crude compound. The crude neutral product was purified by prep-HPLC twice under acidic conditions (Mobile phase: 0.1% TFA in H₂O:ACN; Column: X-BRIDGE-C18 (150*30), 5u; Gradient (% B): 0/35, 8/40, 8.1/98, 10/98, 10.1/35, 13/35; Flow Rate: 20 mL/min; Diluent: ACN+MeOH+H₂O+1% TFA) to obtain (2R,2′R,3S,3′S)-2,2′-((((((2,2′-dimethyl-[1,1′-biphenyl]-3,3′-diyl)bis(methylene))bis(oxy))bis(3-bromo-4,1-phenylene))bis(methylene))bis(azanediyl))bis(3-hydroxybutanoic acid) (85 mg, as the di-TFA salt, yield: 12.7%, HPLC 95.51%) as a white solid. ES−, m/z 810.6 [M−H]; [C₃₈H₄₂Br₂N₂O₈] ¹H NMR (500 MHz, DMSO-d₆) δ 7.70 (s, 2H), 7.54 (d, J=7.5 Hz, 2H), 7.40 (d, J=8.0 Hz, 2H), 7.30 (t, J=6.0 Hz, 4H), 7.10 (d, J=7.5 Hz, 2H), 5.25 (s, 4H), 3.97 (m, 2H), 3.90 (m, 2H), 3.84 (m, 2H), 3.09 (m, 2H), 2.02 (s, 6H), 1.12 (d, J=5.0 Hz, 6H). m.p.=227-231° C.

### Example 19

### (2S,2′S)-1,1′-(((((2,2′-Dimethyl-[1,1′-biphenyl]-3,3′-diyl)bis(methylene))bis(oxy))bis(3-fluoro-4,1-phenylene))bis(methylene))bis(piperidine-2-carboxylic acid) (Cpd 19)

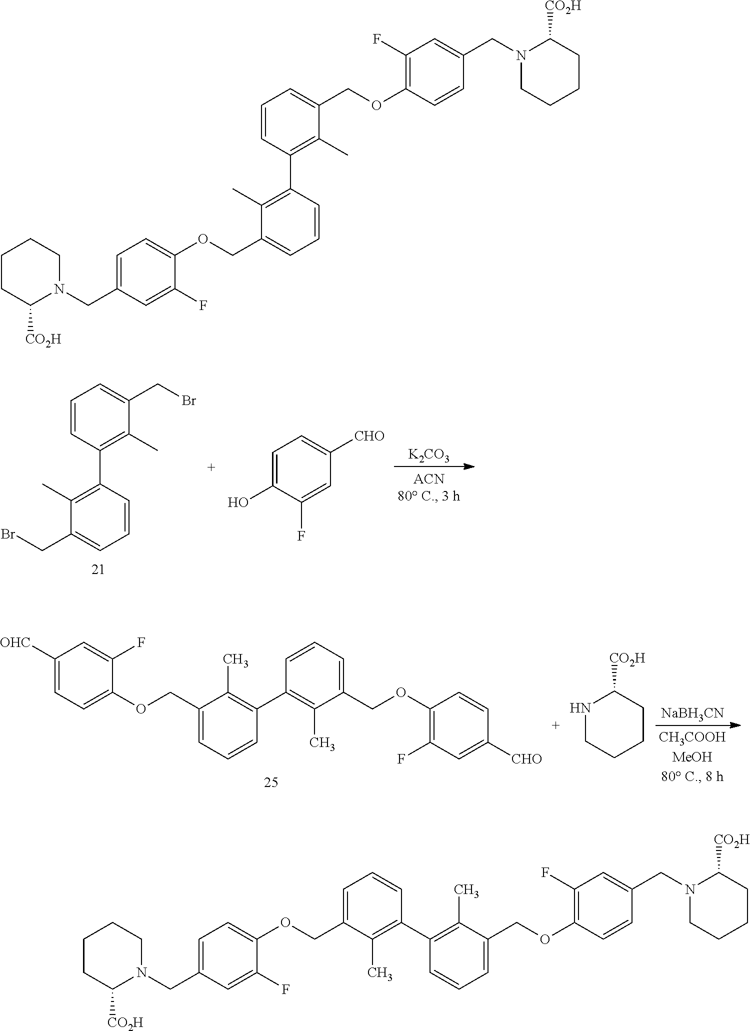

### Step 1

### 4,4′-(((2,2′-Dimethyl-[1,1′-biphenyl]-3,3′-diyl)bis(methylene))bis(oxy))bis(3-fluorobenzaldehyde)(25)

To a stirred solution of 3,3′-bis(bromomethyl)-2,2′-dimethylbiphenyl 21 (1 g, 2.732 mmol) in ACN (20 mL) at room temperature were added K₂CO₃ (1.5 g, 10.928 mmol) and 3-fluoro-4-hydroxybenzaldehyde (1.1 g, 8.196 mmol) and stirred at 80° C. for 3 h. The reaction mixture was concentrated to remove most of the solvent. The residue was poured into ice-water and the resulting precipitate was filtered and dried in vacuum to get 4,4′-(((2,2′-dimethyl-[1,1′-biphenyl]-3,3′-diyl)bis(methylene))bis(oxy))bis(3-fluorobenzaldehyde) (1.1 g, yield: 84%, LC/MS; 93%); ¹H NMR (400 MHz, CDCl₃) δ 9.87 (s, 2H), 7.65-7.62 (m, 4H), 7.45 (d, J=6.8 Hz, 2H), 7.30-7.25 (m, 2H), 7.22-7.15 (m, 4H), 5.26 (s, 4H), 2.07 (s, 6H).

### Step 2

### (2S,2′S)-1,1′-(((((2,2′-Dimethyl-[1,1′-biphenyl]-3,3′-diyl)bis(methylene))bis(oxy))bis(3-fluoro-4,1-phenylene))bis(methylene))bis(piperidine-2-carboxylic acid)

To a solution of 4,4′-(((2,2′-dimethyl-[1,1′-biphenyl]-3,3′-diyl)bis(methylene))bis(oxy))bis(3-fluorobenzaldehyde) (700 mg, 1.439 mmol) in MeOH (15 mL) were added (S)-piperidine-2-carboxylic acid (930 mg, 7.19 mmol) and acetic acid (1 mL) and the reaction mixture was stirred at 80° C. for 4-5 h. The reaction mixture was cooled to room temperature, prior to the addition of NaCNBH₃ (450 mg, 7.19 mmol) and then the reaction mixture was stirred at 80° C. for 3 h. The reaction mixture was poured into ice cold water, and solid thus precipitated was filtered and dried under vacuum. The crude compound was purified by GRACE flash chromatography (40% MeOH in CH₂Cl₂, silica gel) to get (2S,2′S)-1,1′-(((((2,2′-dimethyl-[1,1′-biphenyl]-3,3′-diyl)bis(methylene))bis(oxy))bis(3-fluoro-4,1-phenylene))bis(methylene))bis(piperidine-2-carboxylic acid) (250 mg, yield: 25%, HPLC-98.91%) as a white solid. ES−, m/z 711.0 [M−H]; [C₄₂H₄₆F₂N₂O₆]; ¹H NMR (500 MHz, DMSO-d₆) δ 7.47 (d, J=7.0 Hz, 2H), 7.28 (q, J=7.5 Hz, 4H), 7.20 (dd, J=12.5 Hz, J=10.5 Hz, 2H), 7.11-7.085 (m, 4H), 5.21 (s, 4H), 3.79 (d, J=13.5 Hz, 2H), 3.61-3.47 (m, 2H), 3.44-3.35 (m, 2H), 2.86-2.84 (m, 2H), 2.20-2.19 (m, 2H), 1.99 (s, 6H), 1.79-1.78 (m, 2H), 1.70-1.69 (m, 2H), 1.50-1.48 (m, 6H), 1.47 (m, 2H). m.p.=205-209° C.

### HPLC Conditions:

Column: Gemini-NX 5u C18 (150 mm×4.6 mm, 5 μm)
Mobile phase: 10 mM Ammonium Acetate in H₂O, B: 100% Acetonitrile
Flow rate: 1.0 mL/min
T/% B (min): 0/5, 1.2/5, 3.0/55, 5.0/70, 7/95, 10/95, 12/100, 14.0/5, 16/5.

### Diluent: (ACN:H₂O)

### Temp: 25° C.

Ret. time: 5.06 min

### Purity: 98.9%.

### Example 20

### (2S,2′S)-1,1′-(((((2,2′-Dimethyl-[1,1′-biphenyl]-3,3′-diyl)bis(methylene))bis(oxy))bis(3-methoxy-4,1-phenylene))bis(methylene))bis(piperidine-2-carboxylic acid) (Cpd 20)

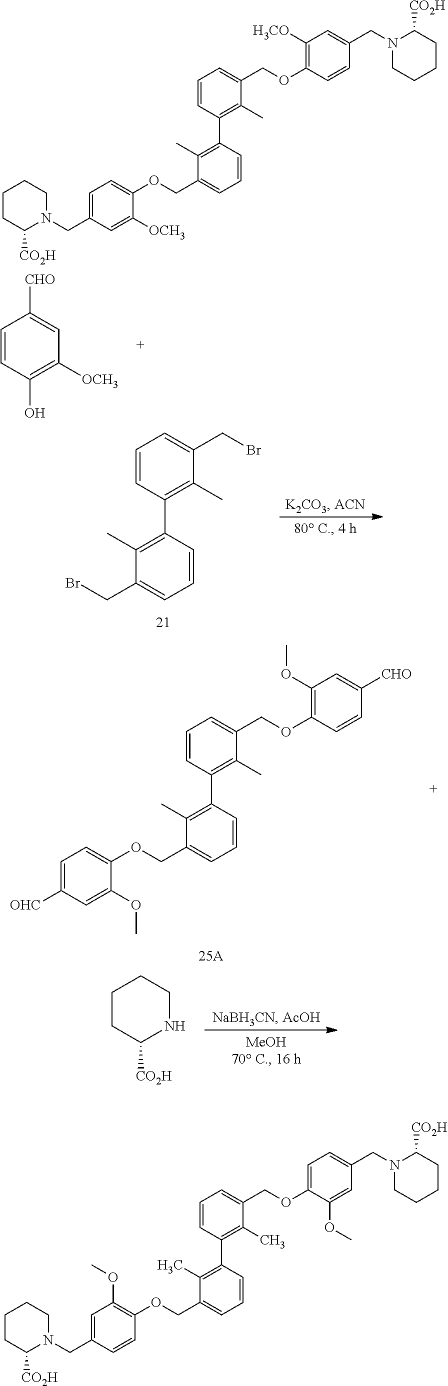

### Step 1

### 4,4′-(((2,2′-Dimethyl-[1,1′-biphenyl]-3,3′-diyl)bis(methylene))bis(oxy))bis(3-methoxybenzaldehyde (25A)

To a stirred solution of 3,3′-bis(bromomethyl)-2,2′-dimethyl-1,1′-biphenyl (21) (700 mg, 1.912 mmol) in ACN (10 mL) at room temperature were added K₂CO₃ (1.05 g, 7.648 mmol) and 4-hydroxy-3-methoxybenzaldehyde (875 mg, 5.737 mmol) and stirred at 80° C. for 3 h. The reaction mixture was concentrated to remove most of the solvent. The residue was poured into ice-water and the resulting precipitate was filtered and dried in vacuum to get 4,4′-(((2,2′-dimethyl-[1,1′-biphenyl]-3,3′-diyl)bis(methylene))bis(oxy))bis(3-methoxybenzaldehyde) (0.9 g, yield: 92%, LC/MS 86%). ES+, m/z 510.8 [M+H]; [C₃₂H₃₀O₆]; ¹H NMR (500 MHz, CDCl₃): δ 9.86 (s, 2H), 7.46-7.43 (m, 6H), 7.28-7.24 (m, 2H), 7.15-7.13 (m, 2H), 7.07 (d, J=8.8 Hz, 2H), 5.25 (s, 4H), 3.94 (s, 6H), 2.07 (s, 6H).

### Step 2

### (2S,2′S)-1,1′-(((((2,2′-dimethyl-[1,1′-biphenyl]-3,3′-diyl)bis(methylene))bis(oxy))bis(3-methoxy-4,1-phenylene))bis(methylene))bis(piperidine-2-carboxylic acid)

To a solution of 4,4′-(((2,2′-dimethyl-[1,1′-biphenyl]-3,3′-diyl)bis(methylene))bis(oxy))bis(3-methoxybenzaldehyde) (750 mg, 1.470 mmol) in MeOH (15 mL) were added (S)-piperidine-2-carboxylic acid (950 mg, 7.352 mmol) and acetic acid (0.5 mL) and the reaction mixture was stirred at 80° C. for 4 h. The reaction mixture was cooled to room temperature, prior to the addition of NaCNBH₃ (460 mg, 7.35 mmol) and then the reaction mixture was stirred at 80° C. for 3 h. The reaction mixture was poured into ice cold water, and solid thus precipitated was filtered and dried under vacuum. The crude compound was purified by GRACE flash chromatography (40% MeOH in CH₂Cl₂, silica gel) to get (2S,2′S)-1,1′-(((((2,2′-dimethyl-[1,1′-biphenyl]-3,3′-diyl)bis(methylene))bis(oxy))bis(3-methoxy-4,1-phenylene))bis(methylene))bis(piperidine-2-carboxylic acid) (200 mg, yield: 20%, HPLC—97.88%) as an off white solid. ES−, m/z 735.0 [M−H]; [C₄₄H₅₂N₂O₈]; ¹H NMR (500 MHz, DMSO-d₆) δ 7.44 (d, J=8.0 Hz, 2H), 7.26 (t, J=7.5 Hz, 2H), 7.07 (d, J=7.5 Hz, 2H), 6.98-6.96 (m, 4H), 6.80 (d, J=8.0 Hz, 2H), 5.08 (s, 4H), 3.86-3.82 (m, 2H), 3.73 (s, 6H), 2.84 (m, 2H), 2.70 (m, 2H), 1.95 (s, 8H), 1.66-1.61 (m, 4H), 1.51 (s, 2H), 1.41 (s, 4H), 1.23 (s, 2H). m.p.=192-196° C.

### HPLC Conditions:

Column: Gemini-NX 5u C18 (150 mm×4.6 mm, 5 μm)
Mobile phase: 10 mM Ammonium Acetate in H₂O, B: 100% Acetonitrile
Flow rate: 1.0 mL/min
T/% B (min): 0/5, 1.2/5, 3.0/55, 5.0/70, 7/95, 10/95, 12/100, 14.0/5, 16/5.

### Diluent: (ACN:H₂O)

### Temp.: 25° C.

Ret. time: 5.01 min

### Purity: 97.88%.

### Example 21

### (2S,2′S)-1,1′-(((((2,2′-Dimethyl-[1,1′-biphenyl]-3,3′-diyl)bis(methylene))bis(oxy))bis(3-hydroxy-4,1-phenylene))bis(methylene))bis(piperidine-2-carboxylic acid) (Cpd 21)

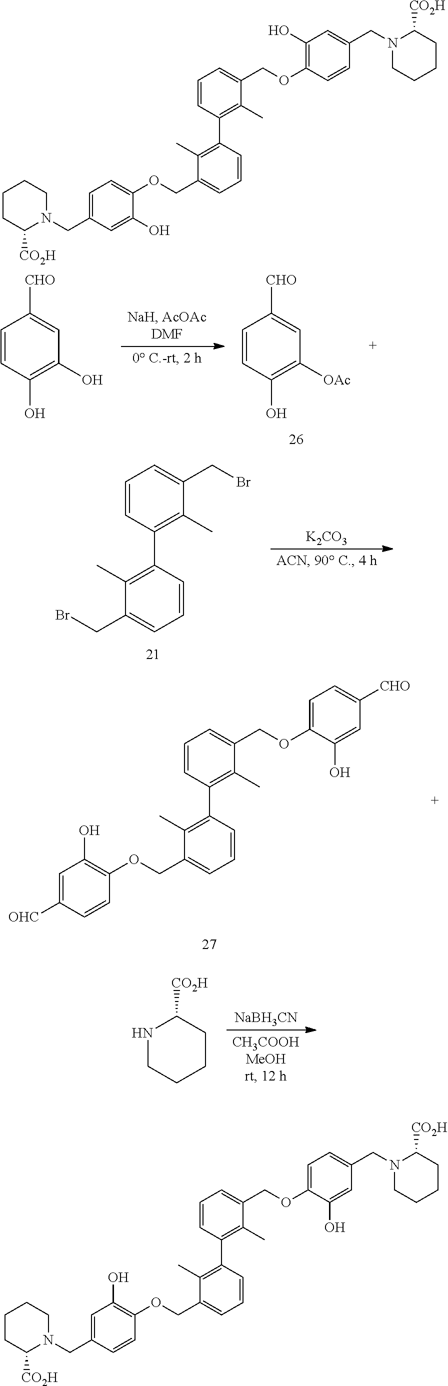

### Step 1

### 5-Formyl-2-hydroxyphenyl acetate (26)

To a solution of 3,4-dihydroxybenzaldehyde (5.0 g, 36.23 mmol) in DMF (35 mL) was added 60% w/w sodium hydride (1.45 g, 61.59 mmol) in portions at 0° C., prior to the slow addition of acetic anhydride (3.6 mL, 38.04 mmol). The resulting reaction mixture was stirred at room temperature for 1 hour. To the reaction mixture was added 2 N aqueous HCl under ice-cooling, and extracted with ethyl acetate. The organic layer was washed with saturated sodium bicarbonate solution and brine, dried over anhydrous Na₂SO₄ and concentrated. The crude product was purified on a silica gel column eluting with pet ether in ethyl acetate (10-50%) to get 5-formyl-2-hydroxyphenyl acetate 26 (5 g, yield: 76%, LC/MS: 72%) as an off-white solid, [C₉H₈O₄]; ¹H NMR (500 MHz, CDCl₃) δ 9.86 (s, 1H), 7.71-7.69 (m, 2H), 7.13 (d, J=11.0 Hz, 1H), 6.01 (s, 1H), 2.40 (s, 3H).

### Step 2

### 4,4′-(((2,2′-Dimethyl-[1,1′-biphenyl]-3,3′-diyl)bis(methylene))bis(oxy))bis(3-hydroxybenzaldehyde (27)

To a stirred solution of 3,3′-bis(bromomethyl)-2,2′-dimethylbiphenyl 21 (1 g, 2.732 mmol) in ACN (25 mL) at room temperature were added K₂CO₃ (1.5 g, 10.928 mmol) and 5-formyl-2-hydroxyphenyl acetate (2.4 g, 13.661 mmol) and stirred at 90° C. for 4 h. The reaction mixture was concentrated to remove most of the solvent. The residue was poured into ice-water and the resulting precipitate was filtered and dried in vacuum to get 4,4′-(((2,2′-dimethyl-[1,1′-biphenyl]-3,3′-diyl)bis(methylene))bis(oxy))bis(3-hydroxybenzaldehyde) 27 (1.1 g, yield: 83%, LC-MS 60%), [C₃₀H₂₆O₆], ¹HNMR (500 MHz, CDCl₃) δ 9.86 (s, 2H), 8.12 (s, 2H), 7.84 (dd, J=13.0 Hz, 8.0 Hz, 2H), 7.53 (d, J=9.5 Hz, 2H), 7.31 (t, J=9.5 Hz, 2H), 7.18 (d, J=9.0 Hz, 2H), 7.14 (d, J=10.5 Hz, 2H), 5.30-5.27 (m, 4H), 2.08 (s, 6H).

### Step 3

### (2S,2′S)-1,1′-(((((2,2′-Dimethyl-[1,1′-biphenyl]-3,3′-diyl)bis(methylene))bis(oxy))bis(3-hydroxy-4,1-phenylene))bis(methylene))bis(piperidine-2-carboxylic acid

To a solution of 4,4′-(((2,2′-dimethyl-[1,1′-biphenyl]-3,3′-diyl)bis(methylene))bis(oxy))bis(3-hydroxybenzaldehyde) 27 (500 mg, 1.037 mmol) in MeOH (20 mL) were added (S)-piperidine-2-carboxylic acid (670 mg, 5.186 mmol) and acetic acid (0.5 mL) and the reaction mixture was stirred at 80° C. for 4-5 h. The reaction mixture was cooled to room temperature, prior to the addition of NaCNBH₃ (321 mg, 5.186 mmol) and then stirred at 80° C. for 4 h. The reaction mixture was poured into ice-cold water, basified with sat NaHCO₃ and solid thus precipitated was filtered and dried under vacuum to get crude compound, which was purified by prep-HPLC (Mobile phase: 0.1% TFA in H₂O: ACN; Column: SYMMETRY-C8 (300*19 MM),75u; Gradient (% B): 0/20, 1/20, 8/50, 10/50, 10.1/98, 12/98, 12.1/1/20, 15/20; Flow Rate: 20 mL/min; Diluent: ACN+THF+H₂O+MeOH) to obtained (2S,2′S)-1,1′-(((((2,2′-dimethyl-[1,1′-biphenyl]-3,3′-diyl)bis(methylene))bis(oxy))bis(3-hydroxy-4,1-phenylene))bis(methylene))bis(piperidine-2-carboxylic acid) (200 mg, yield: 25%, HPLC 97.48%) as a white solid. ES−, m/z 706.9 [M−H]; [C₄₂H₄₈N₂O₈]; ¹H NMR (500 MHz, DMSO-d₆) δ 9.83 (bs, 1H), 9.35 (s, 2H), 7.51 (d, J=7.5 Hz, 2H), 7.29 (t, J=8.0 Hz, 2H), 7.13 (d, J=8.0 Hz, 2H), 7.09 (d, J=7.0 Hz, 2H), 6.94 (d, J=1.5 Hz, 2H), 6.84 (d, J=2.0 Hz, 2H), 5.19-5.14 (m, 4H), 4.33 (d, J=13.5 Hz, 2H), 4.02-3.87 (m, 4H), 3.26-3.16 (m, 2H), 2.90 (bs, 2H), 2.15 (bs, 2H), 2.02 (s, 6H), 1.81-1.41 (m, 10H). m.p.=143-147° C.

### HPLC Conditions:

Column: XBRIDE C18 (150 mm×4.6 mm, 3.5 μm)
Mobile phase: 10 mM Ammonium Acetate in H₂O, B: 100% Acetonitrile
Flow rate: 1.0 mL/min
T/% B (min): 0/5, 1.2/5, 3.0/55, 5.0/70, 7/95, 10/95, 12/100, 14.0/5, 16/5.

### Diluent: ACN:H₂O

### Temp: 25° C.

Ret. time: 4.98 min

### Purity: 99.02%.

### Prep-HPLC Conditions:

Column: SUNFIRE-C18 (300*19 mm), 75u
Mobile phase: 0.1% TFA in H₂O: ACN
Flow rate: 20 mL/min
T/% B (min): 0/20, 1/20, 8/50, 10/50, 10.1/98, 12/98, 12.1/1/20, 15/20

### Diluent: ACN+H₂O+THF+MeOH

### Temp: 25° C.

### Example 22

### 2,2′-((((((2,2′-Dimethyl-[1,1′-biphenyl]-3,3′-diyl)bis(methylene))bis(oxy))bis(2,6-dimethoxy-4,1-phenylene))bis(methylene))bis(azanediyl))bis(ethan-1-ol) (Cpd 22)

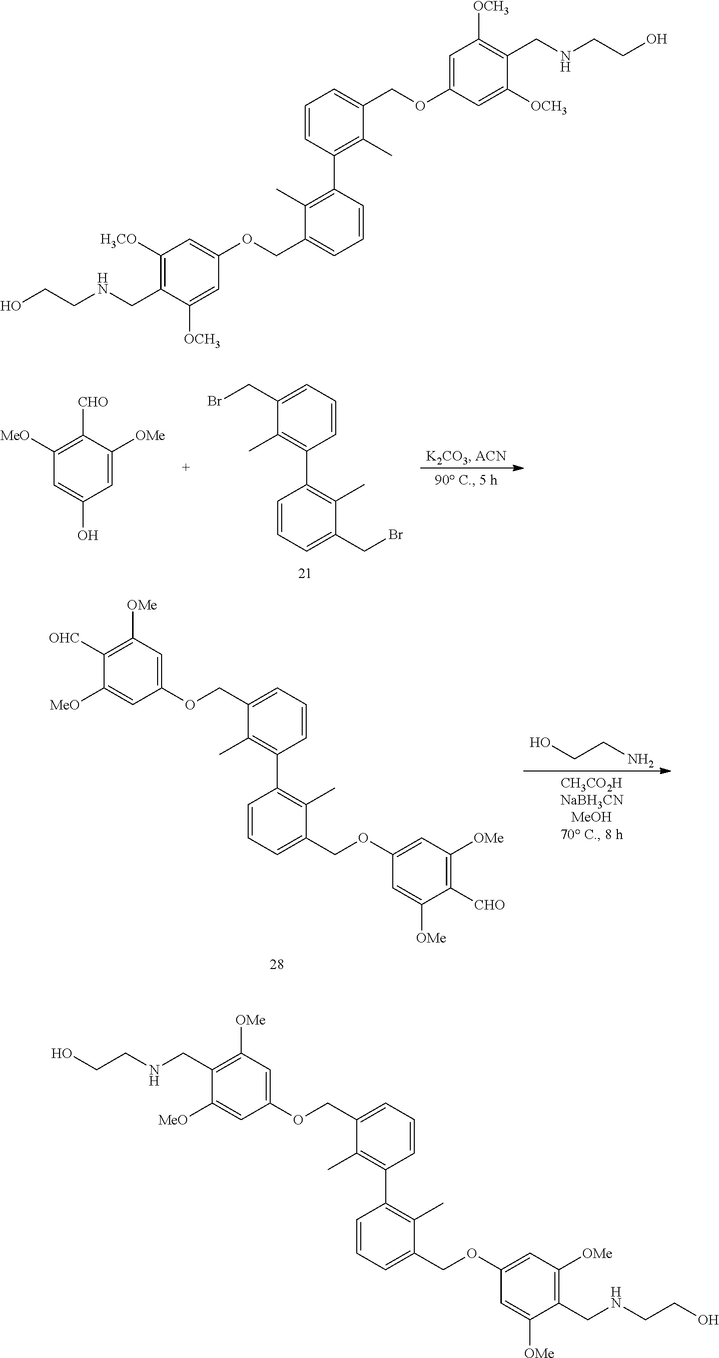

### Step-1

### 4,4′-(((2,2′-Dimethyl-[1,1′-biphenyl]-3,3′-diyl)bis(methylene))bis(oxy))bis(2,6-dimethoxybenzaldehyde)(28)

To a stirred solution of 3,3′-bis(bromomethyl)-2,2′-dimethylbiphenyl (1 g, 2.732 mmol) in ACN (25 mL) at room temperature were added K₂CO₃ (1.5 g, 10.928 mmol) and 4-hydroxy-2,6-dimethoxybenzaldehyde (1.4 g, 8.196 mmol) and stirred at 80° C. for 3 h. The reaction mixture was concentrated to remove most of the solvent. The residue was poured into ice-water and the resulting precipitate was filtered and dried in vacuum to get 4,4′-(((2,2′-dimethyl-[1,1′-biphenyl]-3,3′-diyl)bis(methylene))bis(oxy))bis(2,6-dimethoxybenzaldehyde) (1.0 g, yield: 64%, LC/MS 69%), [C₃₄H₃₄O₈]; ¹H NMR (500 MHz, CDCl₃) δ 10.35 (s, 2H), 7.47-7.45 (m, 2H), 7.30 (t, J=8.0 Hz, 2H), 7.21-7.18 (m, 2H), 6.22 (s, 4H), 5.18 (s, 4H), 3.90 (s, 12H), 2.11 (s, 6H).

### Step 2

### 2-((4-((3′-((4-((2-Hydroxyethyl)amino)-3,5-dimethoxyphenoxy)methyl)-2,2′-dimethyl-[1,1′-biphenyl]-3-yl)methoxy)-2, 6-dimethoxybenzyl)amino)ethan-1-ol

To a solution of 4,4′-(((2,2′-dimethyl-[1,1′-biphenyl]-3,3′-diyl)bis(methylene))bis(oxy))bis(2,6-dimethoxybenzaldehyde) (900 mg, 1.578 mmol) in MeOH (20 mL) were added 2-aminoethanol (482 mg, 7.89 mmol) and acetic acid (0.5 mL) and the reaction mixture was stirred at 80° C. for 4-5 h. The reaction mixture was cooled to room temperature, prior to the addition of NaCNBH₃ (495 mg, 7.89 mmol) and then stirred at 80° C. for 3 h. The reaction mixture was poured into ice cold water, and solid thus precipitated was filtered and dried under vacuum. The crude compound was purified by GRACE flash chromatography (40% MeOH in CH₂Cl₂, silica gel) to get 2-((4-((3′-((4-((2-hydroxyethyl)amino)-3,5-dimethoxyphenoxy)methyl)-2,2′-dimethyl-[1,1′-biphenyl]-3-yl)methoxy)-2,6-dimethoxybenzyl)amino)ethan-1-ol (190 mg, yield: 25%, HPLC 95.45%) as an off white solid. ES−, m/z 660.9 [M−H]; [C₃₇H₄₆N₂O₈]; ¹H NMR (500 MHz, DMSO-d₆) δ 7.49 (d, J=7.5 Hz, 2H), 7.29 (t, J=8.0 Hz, 2H), 7.11 (d, J=7.0 Hz, 2H), 6.39 (s, 4H), 5.16 (q, J=12.0 Hz, 4H), 4.38 (bs, 2H), 3.75 (s, 12H), 3.60 (s, 4H), 3.41-3.39 (m, 4H), 2.49-2.47 (m, 4H), 2.15 (s, 6H). m.p.=86-90° C.

### HPLC Conditions:

Column: Acquity UPLC BEH C18 (100 mm×2.1 mm, 1.7 μm)
Mobile phase: A: 0.025% TFA in CAN, B: 0.025% TFA in Water
Flow rate: 0.4 mL/min
T/% B (min): 0/10, 1.5/10, 8/98, 12/98, 12.01/10.

### Diluent: (ACN:H₂O 70:30)

### Temp: 25° C.

Ret. time: 5.54 min

### Purity: 95.45%.

### General Synthetic Method for Examples 23-25

To a mixture of the Cpd 1 (52 mg, 0.062 mmol), HATU (95 mg, 0.25 mmol, 4 eq.) in dry NMP (1.5 mL), was added DIEA (0.1 mL). The resulting reaction mixture was stirred at room temperature for 30 minutes. To above reaction mixture, was added corresponding amine (0.25 mmol, 4 eq.). The reaction was continued to stir at r.t. over night. After completion of reaction as indicated by LCMS of reaction mixture, water (30 mL) was added. The aqueous mixture was extracted with EtOAc (30 mL×2). The combined organic extractions were washed with water (20 mL×2), brine, dried (Na₂SO₄) and concentrated. The residue was treated with water (5 mL). The product solidified and was collected by filtration. The solid product was further washed with water (3 mL), dried in vacuum thoroughly to afford the desired amide compound. The compound was usually pure as confirmed by LCMS and NMR analysis, or further purified by silica gel column or preparative TLC plate chromatography.

### Example 23

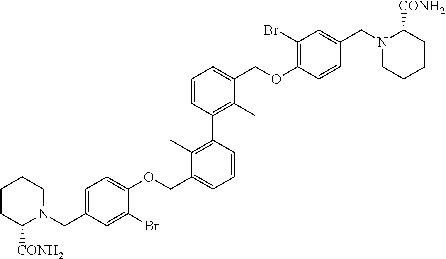 [SMILES: Cc1c(COc2ccc(CN3CCCC[C@H]3C(N)=O)cc2Br)cccc1-c1cccc(COc2ccc(CN3CCCC[C@H]3C(N)=O)cc2Br)c1C]

### (2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(piperidine-2-carboxamide) (Cpd 23)

This compound was prepared from Cpd 1 and ammonium chloride and obtained as a gray solid (39 mg, yield 78.3%). ES+, m/z 833.0 [M+1]; ¹H NMR (500 MHz, DMSO-d₆), δ 7.60 (brs, 2H), 7.54 (d, J=8.0 Hz, 2H) 7.32-7.28 (m, 6H), 7.22 (d, J=8.5 Hz, 2H), 7.10 (d, J=7.5 Hz, 2H), 6.99 (brs, 2H), 5.23 (s, 4H), 3.69 (d, J=13.5 Hz, 2H), 3.04 (d, J=13.5 Hz, 2H), 2.74-2.72 (m, 2H), 2.65-2.63 (m, 2H), 2.03 (s, 6H), 1.88-1.85 (m, 2H), 1.74-1.72 (m, 2H), 1.66-1.63 (m, 2H), 1.59-1.54 (m, 2H), 1.51-1.48 (m, 2H), 1.37-1.34 (m, 2H), 1.24-1.19 (m, 2H).

### Example 24

### (2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(N-(2-hydroxyethyl)piperidine-2-carboxamide) (Cpd 24)

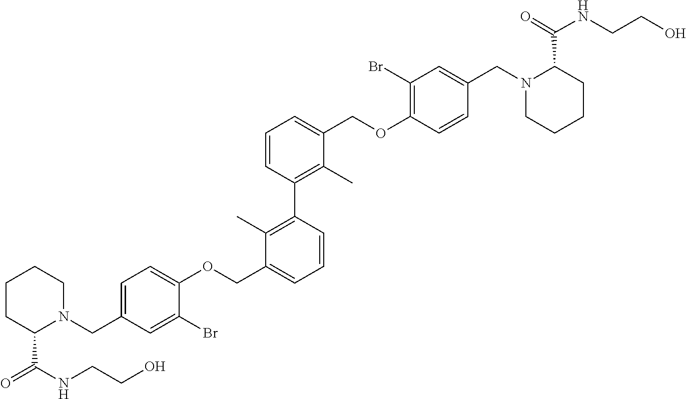 [SMILES: Cc1c(COc2ccc(CN3CCCC[C@H]3C(=O)NCCO)cc2Br)cccc1-c1cccc(COc2ccc(CN3CCCC[C@H]3C(=O)NCCO)cc2Br)c1C]

This compound was prepared from Cpd 1 and 2-aminoethanol and obtained as a pale yellow solid (56 mg, yield 98%). ES+, m/z 921.5 [M+1]; ¹H NMR (500 MHz, DMSO-d₆), δ 7.76 (t, J=5.5 Hz, 2H), 7.59 (s, 2H), 7.54 (d, J=7.0 Hz, 2H), 7.31 (d, J=7.5 Hz, 2H), 7.30 (t, J=7.5 Hz, 2H), 7.22 (d, J=8.5 Hz, 2H), 7.10 (d, J=8.0 Hz, 2H), 5.23 (s, 4H), 4.66 (t, J=5.5 Hz, 2H), 3.66 (d, J=13.5 Hz, 2H), 3.42 (q, J=5.5 Hz, 4H), 3.25-3.21 (m, 2H), 3.15-3.11 (m, 2H), 3.02 (d, J=13.0 Hz, 2H), 2.71-2.69 (m, 2H), 2.69-2.66 (m, 2H), 2.34 (m, 4H), 2.03 (s, 4H), 1.87 (t, J=5.5 Hz, 2H), 1.73-1.71 (m, 2H), 1.66-1.63 (m, 2H), 1.53-1.49 (m, 4H), 1.37-1.30 (m, 2H), 1.23-1.18 (m, 2H).

### Example 25

### (2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(N-(2-(dimethylamino)ethyl)piperidine-2-carboxamide) (Cpd 25)

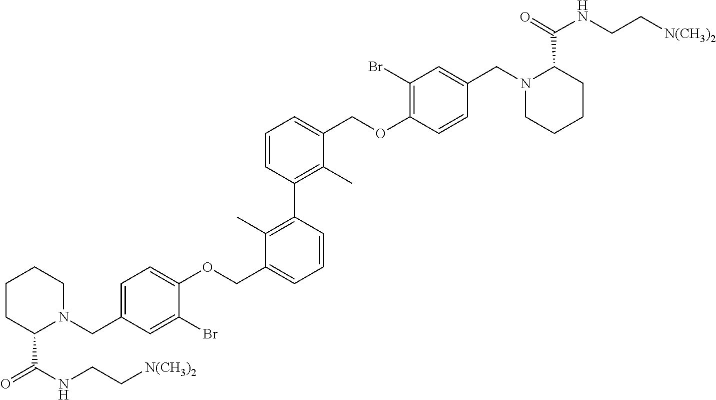 [SMILES: Cc1c(COc2ccc(CN3CCCC[C@H]3C(=O)NCCN(C)C)cc2Br)cccc1-c1cccc(COc2ccc(CN3CCCC[C@H]3C(=O)NCCN(C)C)cc2Br)c1C]

The compound was prepared from Cpd 1 and N¹,N¹-dimethylethane-1,2-diamine and obtained as a pale orange solid (48 mg, yield 82%). ES+, m/z 975.7 [M+1]; ¹H NMR (500 MHz, DMSO-d₆), δ 7.65 (brs, 2H), 7.61 (s, 2H), 7.25 (d, J=7.0 Hz, 2H), 7.31-7.29 (m, 4H), 7.22 (d, J=8.0 Hz, 2H), 7.10 (d, J=7.5 Hz, 2H), 5.24 (s, 4H), 3.72 (d, J=12.5 Hz, 2H), 3.09 (m, 2H), 2.99 (d, J=12.5 Hz, 2H), 2.71 (d, J=10.5 Hz, 2H), 2.64 (d, J=10.0 Hz, 2H), 2.34 (m, 4H), 2.13 (s, 12H), 2.02 (s, 6H), 1.86 (m, 2H), 1.74 (m, 2H), 1.64 (m, 2H), 1.50 (m, 4H), 1.35 (m, 2H), 1.23-1.15 (m, 4H).

### Example 26

### (2S,2′S)-1,1′-(((((2,2′-Dimethyl-[1,1′-biphenyl]-3,3′-diyl)bis(methylene))bis(oxy))bis(3-ethynyl-4,1-phenylene))bis(methylene))bis(piperidine-2-carboxylic acid) di-trifluoroacetic acid salt (Cpd 26)

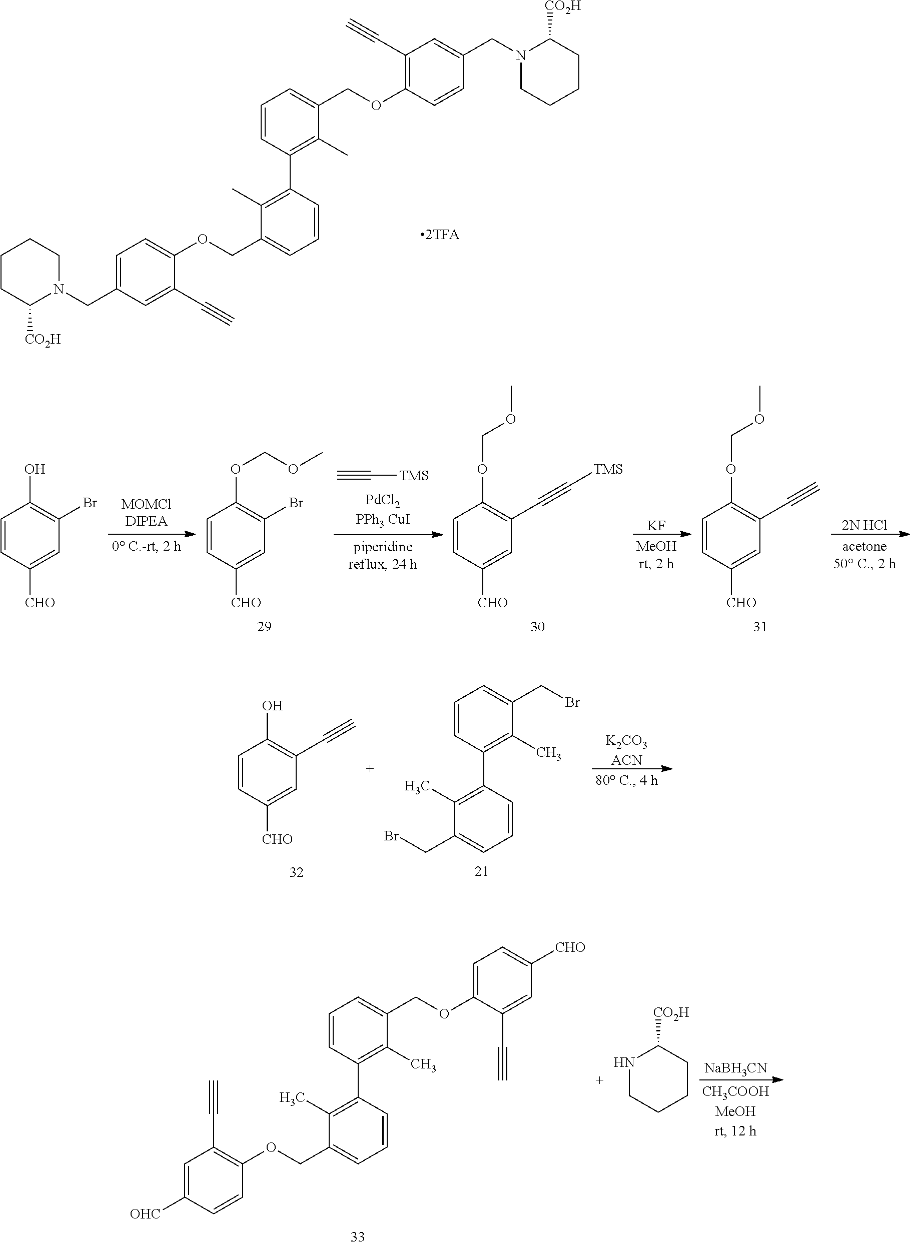

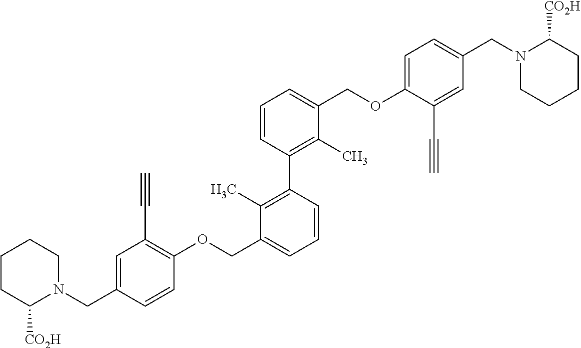 [SMILES: C#Cc1cc(CN2CCCC[C@H]2C(=O)O)ccc1OCc1cccc(-c2cccc(COc3ccc(CN4CCCC[C@H]4C(=O)O)cc3C#C)c2C)c1C]

### Step 1

### 3-Bromo-4-(methoxymethoxy)benzaldehyde (29)

To a solution of 3-bromo-4-hydroxybenzaldehyde (5 g, 24.875 mmol) in CH₂Cl₂ (50 mL) were added DIPEA (8.8 g, 49.75 mmol) and chloro(methoxy)methane (2.8 g, 37.31 mmol) at 0° C. and stirred the resulting solution at room temperature for 2 h. The reaction mixture was poured into ice-cold water and extracted with CH₂Cl₂ (2×100 mL). The combined organic layer was dried over Na₂SO₄ and concentrated to give the crude product, which was purified on a silica gel column, eluting with ethyl acetate in pet-ether (0 to 20%) to get 3-bromo-4-(methoxymethoxy)benzaldehyde (3.8 g, 63%) as an off-white solid, [C₉H₉BrO₃]; ¹H NMR (400 MHz, CDCl₃): δ 9.86 (s, 1H), 8.09 (s, 1H), 7.80-7.77 (dd, J=8.4, 2 Hz, 1H), 7.27 (d, J=9.5 Hz, 1H), 2.04 (s, 2H), 3.53 (s, 3H).

### Step 2

### 4-(Methoxymethoxy)-3-((trimethylsilyl)ethynyl)benzaldehyde (30)

A mixture of 3-bromo-4-(methoxymethoxy)benzaldehyde (3.8 g, 15.57 mmol), ethynyltrimethylsilane (9.2 mL, 62.29 mmol) and CuI (30 mg, 0.124 mmol) in piperidine (70 mL) was degassed with argon for 30 min. PdCl₂ (56 mg, 0.38 mmol) and PPh₃ (56 mg, 0.38 mmol) were added and again degassed with argon for 30 min and the reaction mixture was maintained at reflux for 24 h. Water (100 mL) was added to the reaction mixture to induce precipitation which was filtered to get the crude product. The crude product was purified by flash chromatography (silica; pet-ether/ethyl acetate 0/100 to 30/70) to afford to get 4-(methoxymethoxy)-3-((trimethylsilyl)ethynyl)benzaldehyde (2.8 g, 70%, LC/MS, 81%) as a solid ES+, m/z 263 [M+H]; [C₁₄H₁₈O₃ Si]; ¹H NMR (500 MHz, CDCl₃): δ 9.86 (s, 1H), 7.97 (s, 1H), 7.80-7.78 (dd, J=8.5.2 Hz, 1H), 7.21-7.20 (d, J=9 Hz, 1H), 5.32 (s, 2H), 3.53 (s, 3H), 0.27 (s, 9H).

### Step 3

### 3-Ethynyl-4-(methoxymethoxy)benzaldehyde (31)

To a solution of 4-(methoxymethoxy)-3-((trimethylsilyl)ethynyl)benzaldehyde (3.3 g, 12.59 mmol) in MeOH (30 mL) was added KF (1.46 g, 25.19 mmol) at room temperature and stirred for 5 h. The reaction mixture was concentrated under reduced pressure, the residue was partitioned between water (20 mL) and ethyl acetate (20 mL); the organic layer was separated and concentrated under reduced pressure to afford 3-ethynyl-4-(methoxymethoxy)benzaldehyde as a yellow solid (1.38 g, yield=57%), [C₁₁H₁₀O₃]; ¹H NMR (500 MHz, CDCl₃): δ 9.88 (s, 1H), 8.00-7.99 (m, 1H), 7.84-7.82 (dd, J=9.0, 9.0 Hz, 1H), 7.28 (s, 1H), 5.35 (s, 2H), 3.53 (s, 3H), 3.34 (s, 1H).

### Step 4

### 3-Ethynyl-4-hydroxybenzaldehyde (32)

To a solution of 3-ethynyl-4-(methoxymethoxy)benzaldehyde (1.38 g, 7.263 mmol) in MeOH (15 mL) was added 2N HCl (5 mL) and stirred at 50° C. for 2 h. The reaction mixture was concentrated under reduced pressure, the residue was partitioned between water (20 mL) and ethyl acetate (20 mL); the organic layer was separated and concentrated under reduced pressure to afford 3-ethynyl-4-hydroxybenzaldehyde as a colorless solid (1 g, LC/MS, 55%), ES+, m/z 147 [M+H]; [C₉H₆O₂]; ¹H NMR (400 MHz, CDCl₃): δ 9.86 (s, 1H), 7.94 (s, 1H), 7.83 (d, J=6.0 Hz, 1H), 7.08 (d, J=7.4 Hz, 1H), 6.37 (s, 1H), 3.55 (s, 1H).

### Step 5

### 4,4′-(((2,2′-Dimethyl-[1,1′-biphenyl]-3,3′-diyl)bis(methylene))bis(oxy))bis(3-ethynylbenzaldehyde) (33)

To a stirred solution of 3,3′-bis(bromomethyl)biphenyl (500 mg, 1.366 mmol) in ACN (30 mL) at room temperature were added 3-ethynyl-4-hydroxybenzaldehyde (797 mg, 5.46 mmol) and K₂CO₃ (754 g, 5.46 mmol) and reaction mixture was stirred at 80° C. for 4 h. The reaction mixture was cooled to room temperature, diluted with cold water (50 mL) and extracted with EtOAc (2×50 mL). The combined organic layer was washed with brine (2×50 mL), dried over anhydrous Na₂SO₄, filtered and concentrated under reduced pressure to afford 4,4′-(((2,2′-dimethyl-[1,1′-biphenyl]-3,3′-diyl)bis(methylene))bis(oxy))bis(3-ethynylbenzaldehyde) (0.7 g, LC/MS, 55%) as a light green solid; [C₃₄H₂₆O₄]; ¹H NMR (500 MHz, CDCl₃) δ 9.85 (s, 2H), 8.02 (m, 2H), 7.88 (dd, J=13.5 Hz, 8.5 Hz, 2H), 7.52 (d, J=9.5 Hz, 2H), 7.29 (d, J=9.5 Hz, 2H), 7.16-7.11 (m, 4H), 5.30-5.25 (m, 4H), 3.32 (s, 2H), 2.05 (s, 6H).

### Step 6

### (2S,2′S)-1,1′-(((((2,2′-Dimethyl-[1,1′-biphenyl]-3,3′-diyl)bis(methylene))bis(oxy))bis(3-ethynyl-4,1-phenylene))bis(methylene))bis(piperidine-2-carboxylic acid) di-trifluoroacetic acid salt

To a solution of 4,4′-(((2,2′-dimethyl-[1,1′-biphenyl]-3,3′-diyl)bis(methylene))bis(oxy))bis(3-ethynylbenzaldehyde) (700 mg, 1.405 mmol) in MeOH (20 mL) were added (S)-piperidine-2-carboxylic acid (910 mg, 7.02 mmol) and AcOH (0.5 mL) and stirred at 80° C. for 4 h. The reaction mixture was cooled to room temperature, prior to the addition of NaCNBH₃ (440 mg, 7.026 mmol) and stirred at 80° C. for 3 h. The reaction mass was poured into ice-cold water, basified with sat. NaHCO₃ and solid thus precipitated was filtered and dried under vacuum to get crude compound. The crude neutral product was purified by prep-HPLC under acidic conditions (Mobile phase: 0.1% TFA in H₂O: ACN; Column: Symmetry-C8 (300*19 MM), 7u; Gradient: (T % B): 0/30, 8/50, 8.1/30, 11/30; Flow Rate: 20 mL/min; Diluent: ACN+H₂O+THF+MeOH) to get (2S,2′S)-1,1′-(((((2,2′-dimethyl-[1,1′-biphenyl]-3,3′-diyl)bis(methylene))bis(oxy))bis(3-ethynyl-4, 1-phenylene))bis(methylene))bis(piperidine-2-carboxylic acid) as the di-trifluoroacetic acid salt (150 mg, yield: 20%, HPLC 97.14%) as a pale yellow solid. ES−, m/z 723.3 [M−H]; [C₄₆H₄₈N₂O₆]; δ 10.01 (bs, 2H), 7.54 (d, J=8.5 Hz, 4H), 7.47 (dd, J=10.5, 7.0 Hz, 2H), 7.35-7.29 (m, 4H), 7.11 (d, J=8.0 Hz, 2H), 5.27 (s, 4H), 4.38-4.33 (m, 4H), 4.08 (m, 2H), 3.89 (m, 2H) 3.25 (m, 2H), 2.91 (m, 2H), 2.15 (d, J=10.5 Hz, 2H), 2.03 (s, 6H), 1.72-1.67 (m, 8H), 1.51 (m, 2H). m.p.=175° C. (decomp.)

### HPLC Conditions:

Column: XBRIDE C18 (150 mm×4.6 mm, 3.5 μm)
Mobile phase: 10 mM Ammonium Acetate in H₂O, B: 100% Acetonitrile
Flow rate: 1.0 mL/min
T/% B (min): 0/5, 1.2/5, 3.0/55, 5.0/70, 7/95, 10/95, 12/100, 14.0/5, 16/5.

### Diluent: (ACN:H₂O)

### Temp: 25° C.

Ret. time: 5.20 min

### Purity: 97.14%.

### Prep-HPLC Conditions:

### Column: Symmetry-C8 (300*19 MM), 7u

Mobile phase: 0.1% TFA in H₂O: ACN
Flow rate: 25 mL/min
T/% B (min): 0/30, 8/50, 8.1/30, 11/30.

### Diluent: ACN+H₂O+THF+MeOH

### Temp: 25° C.

### Example 27

### (2S,2′S)-1,1′-(((((2,2′-Dimethyl-[1,1′-biphenyl]-3,3′-diyl)bis(methylene))bis(oxy))bis(3-ethyl-4,1-phenylene))bis(methylene))bis(piperidine-2-carboxylic acid) di-trifluoroacetic acid salt (Cpd 27)

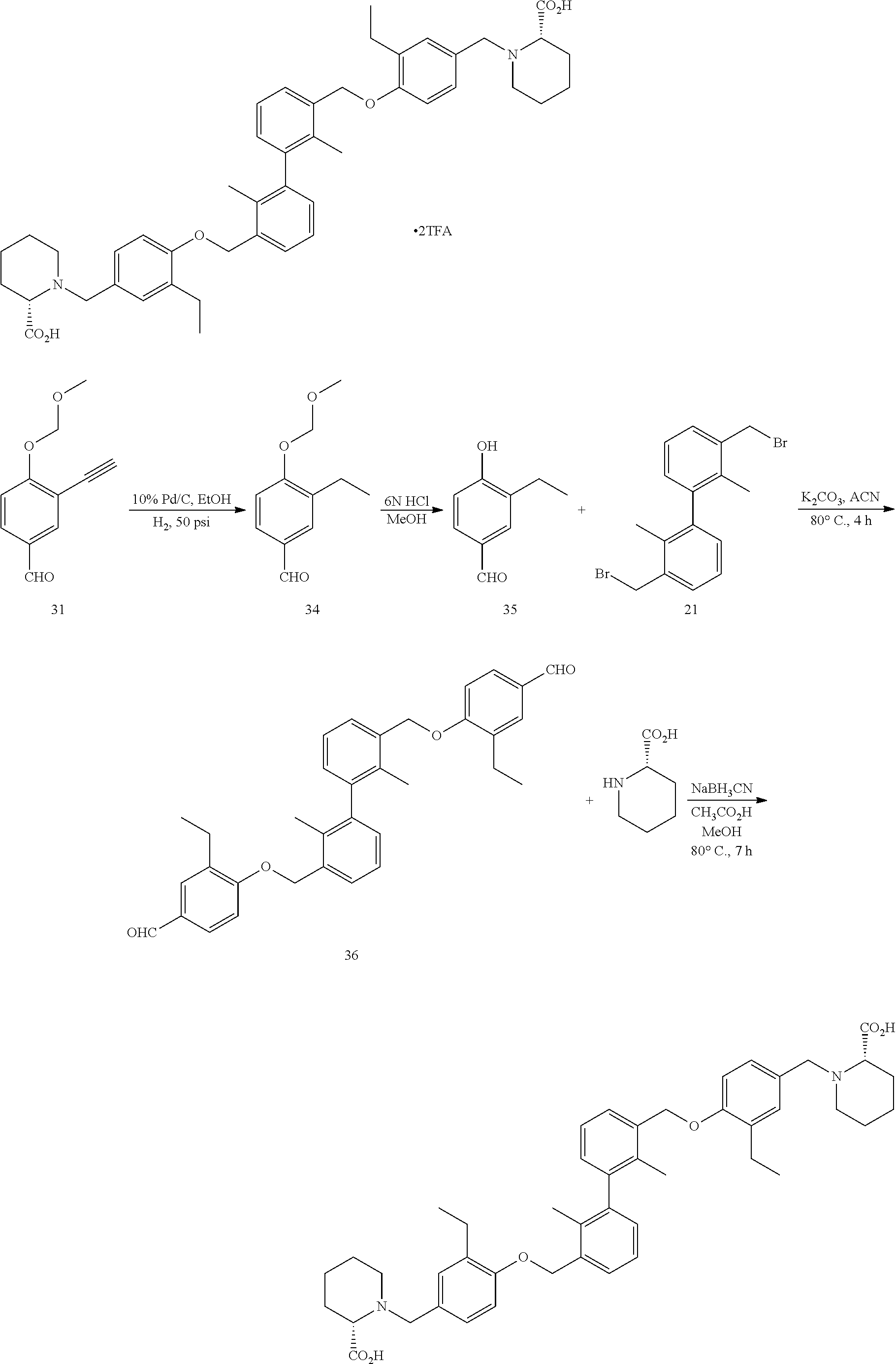

### Step 1

### 3-Ethyl-4-(methoxymethoxy)benzaldehyde (34)

To a solution of 3-ethynyl-4-(methoxymethoxy)benzaldehyde (1 g, 5.263 mmol) in ethanol (50 mL) was added 10% Pd/C (160 mg) in a Parr shaker. The reaction mixture was stirred at room temperature for 12 h under hydrogen (50 psi). The reaction mixture was filtered through celite pad and the filtrate was evaporated to give a crude product, which was in turn was purified by GRACE flash chromatography, eluting with pet-ether: EtOAc (80:20) to afford 3-ethyl-4-(methoxymethoxy)benzaldehyde (600 mg, 60%, LC/MS, 90%) as an off white solid. ES+, m/z 194.09 [M+H]; [C₁₁H₁₄O₃]; ¹H NMR (400 MHz, CDCl₃): δ 9.88 (s, 1H), 7.72-7.67 (m, 2H), 7.17 (d, J=10.5 Hz, 1H), 5.29 (s, 2H), 3.49 (s, 3H), 2.72 (q, J=9.5 Hz, 2H), 1.26-1.22 (t, J=7.6 Hz, 3H).

### Step 2

### 3-Ethyl-4-hydroxybenzaldehyde (35)

To a solution of 3-ethyl-4-(methoxymethoxy)benzaldehyde (600 mg, 3.07 mmol) in MeOH (10 mL) at room temperature, 6N HCl (3 mL) was added slowly. The reaction mixture was stirred at room temperature for 2 h. The reaction mixture was diluted with CH₂Cl₂ (100 mL), washed with water (20 mL×2), brine, dried over Na₂SO₄, and concentrated to provide the crude product, which was then purified on a silica gel column, eluting with ethyl acetate in pet ether (20:80) to get 3-ethyl-4-hydroxybenzaldehyde as a colorless liquid (440 mg, yield=96%), ES+, m/z 151 [M+H]; [C₉H₁₀O₂]; ¹H NMR (500 MHz, CDCl₃): δ 9.84 (s, 1H), 7.72 (d, J=1.5 Hz, 1H), 7.65 (dd, J=8.0, 2.0 Hz, 1H), 6.91 (d, J=8.0 Hz, 1H), 6.17 (bs, 1H), 2.71 (q, J=7.5 Hz, 2H), 1.29-1.22 (t, J=7.5 Hz, 3H).

### Step 3

### 4,4′-(((2,2′-Dimethyl-[1,1′-biphenyl]-3,3′-diyl)bis(methylene))bis(oxy))bis(3-ethylbenzaldehyde (36)

To a stirred solution of 3,3′-bis(bromomethyl)-2,2′-dimethyl-1,1′-biphenyl (500 mg, 1.363 mmol) in ACN (15 mL) at room temperature were added 3-ethyl-4-hydroxybenzaldehyde (602 mg, 4.133 mmol) and K₂CO₃ (750 mg, 5.455 mmol) and reaction mixture was stirred at 80° C. for 4 h. The reaction mixture was cooled to room temperature, diluted with cold water (30 mL) and extracted with EtOAc (2×50 mL). The combined organic layer was washed with brine (2×50 mL), dried over anhydrous Na₂SO₄, filtered and concentrated under reduced pressure to afford 4,4′-(((2,2′-dimethyl-[1,1′-biphenyl]-3,3′-diyl)bis(methylene))bis(oxy))bis(3-ethylbenzaldehyde) (540 mg, yield: 24%, LC/MS: 81%) as an off white solid. ES+, m/z 507 [M+H]; [C₃₄H₃₄O₄]; ¹H NMR (400 MHz, CDCl₃): δ 9.89 (s, 2H), 7.75-7.73 (m, 4H), 7.47 (d, J=9.0 Hz, 2H), 7.30 (t, J=9.5 Hz, 2H), 7.18 (d, J=8.5 Hz, 2H), 7.08 (d, J=11.0 Hz, 2H), 5.21 (s, 4H), 2.75 (q, J=9.0 Hz, 4H), 2.07 (s, 6H), 1.24 (t, J=9.5 Hz, 6H).

### Step 4

### (2S,2′S)-1,1′-(((((2,2′-Dimethyl-[1,1′-biphenyl]-3,3′-diyl)bis(methylene))bis(oxy))bis(3-ethyl-4,1-phenylene))bis(methylene))bis(piperidine-2-carboxylic acid) di-trifluoroacetic acid

To a solution of 4,4′-(((2,2′-dimethyl-[1,1′-biphenyl]-3,3′-diyl)bis(methylene))bis(oxy))bis(3-ethylbenzaldehyde) (540 mg, 1.066 mmol) in MeOH (20 mL) were added (S)-piperidine-2-carboxylic acid (700 mg, 5.33 mmol) and AcOH (0.5 mL) and stirred at 80° C. for 4 h. The reaction mixture was cooled to room temperature, prior to the addition of NaCNBH₃ (332 mg, 5.339 mmol) and stirred at 80° C. for 3 h. The reaction mass was poured into ice-cold water, basified with sat. NaHCO₃ and solid thus precipitated was filtered and dried under vacuum to get crude compound. The crude neutral product was purified by prep-HPLC under acidic conditions (Mobile phase: 0.1% TFA in H₂O:ACN; Column: INERTSIL ODS-C18 (19*120 MM), 5u; Gradient: (T % B): 0/35, 8/60, 9/60, 9.1/98, 12/98, 12.1/35, 15/35; Flow Rate: 20 mL/min; Diluent: ACN+H₂O+THF+MeOH) to get (2S,2′S)-1,1′-(((([1,1′-biphenyl]-3,3′-diylbis(methylene))bis(oxy))bis(3-bromo-4,1-phenylene))bis(methylene))bis(piperidine-2-carboxylic acid) as the di-trifluoroacetic acid salt (340 mg, yield: 43%, HPLC 97.68%) as a white solid. ES−, m/z 731.4 [M−H]; [C₄₆H₅₆N₂O₆]; ¹H NMR (500 MHz, DMSO-d₆) δ 7.50 (d, J=7.0 Hz, 2H), 7.33-7.26 (m, 6H), 7.21 (d, J=10.0 Hz, 2H), 7.11 (d, J=6.5 Hz, 2H), 5.23-5.17 (m, 4H), 4.36 (d, J=13.0 Hz, 2H), 4.15-4.09 (m, 2H), 3.91-3.81 (m, 2H), 3.61-3.39 (m, 2H), 3.39 (m, 2H), 2.95-2.85 (m, 2H), 2.63-2.59 (m, 4H), 2.14 (m, 2H), 2.02 (s, 6H), 1.72-1.66 (m, 8H), 1.51-1.49 (m, 2H), 1.15 (t, J=5.5 Hz, 6H). m.p.=131-135° C.

### HPLC Conditions:

Column: XBRIDE C18 (150 mm×4.6 mm, 3.5 μm)
Mobile phase: 10 mM Ammonium Acetate in H₂O, B: 100% Acetonitrile
Flow rate: 1.0 mL/min
T/% B (min): 0/5, 1.2/5, 3.0/55, 5.0/70, 7/95, 10/95, 12/100, 14.0/5, 16/5.

### Diluent: ACN:H₂O

### Temp.: 25° C.

Ret. time: 5.56 min

### Purity: 97.68%.

### PreP-HPLC Conditions:

### Column: INERTSIL ODS-C18 (19*120 MM), 5u

Mobile phase: 0.1% TFA in H₂O: ACN
Flow rate: 20 mL/min
T/% B (min): 0/35, 8/60, 9/60, 9.1/98, 12/98, 12.1/35, 15/35.

### Diluent: (ACN+H₂O+THF+MeOH)

### Temp.: 25° C.

### Example 28

### (2S,2′S)-1,1′-(((((2,2′-Dimethyl-[1,1′-biphenyl]-3,3′-diyl)bis(methylene))bis(oxy))bis(3-(methylthio)-4,1-phenylene))bis(methylene))bis(piperidine-2-carboxylic acid) di-trifluoroacetic acid salt (Cpd 28)

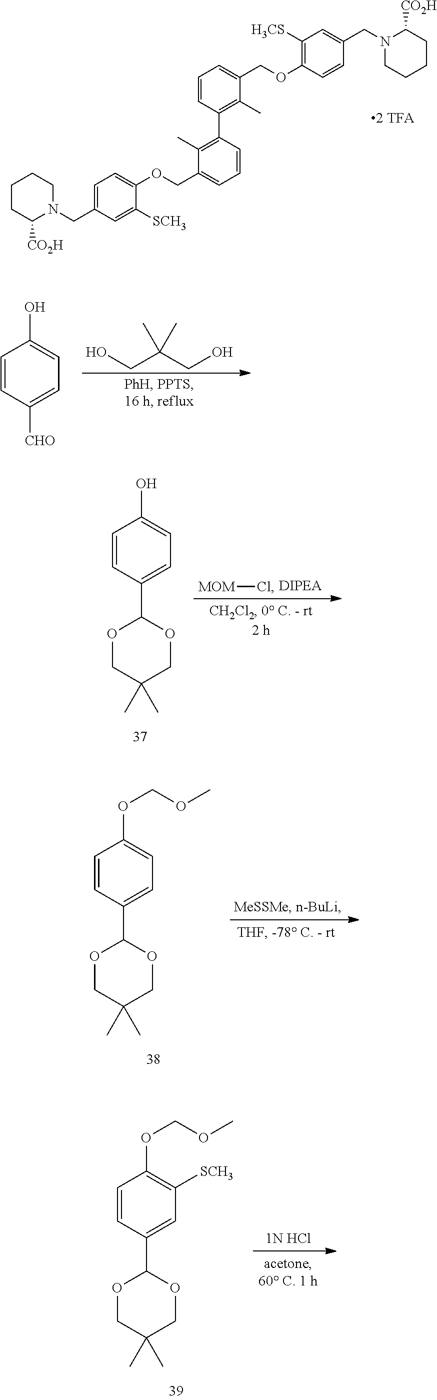

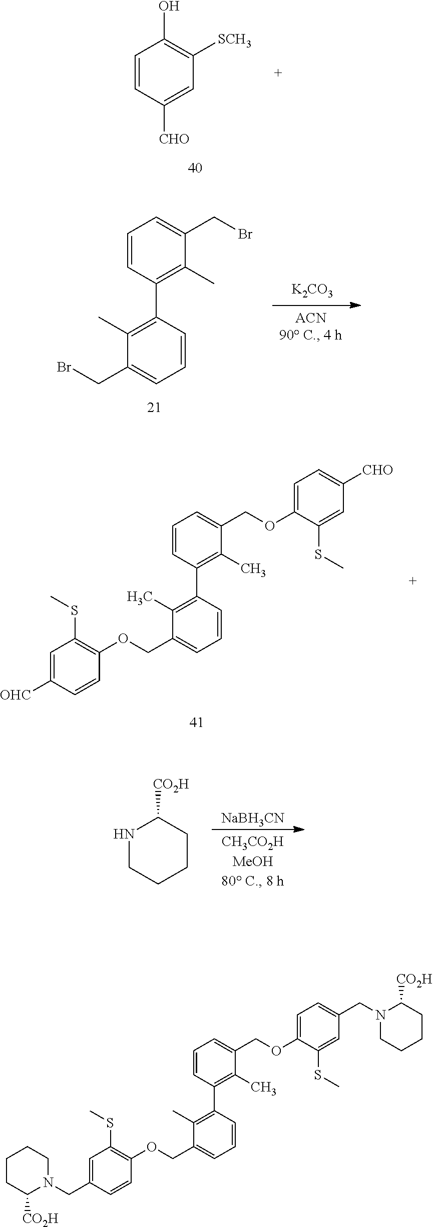

### Step 1

### 4-(5,5-Dimethyl-1,3-dioxan-2-yl)phenol (37)

To a stirred solution of 4-hydroxybenzaldehyde (5 g, 40.98 mmol) in benzene (50 mL) at room temperature were added neo-pentyl glycol (4.68 g, 45.08 mmol) and pyridinium p-toluenesulfonate (0.5 g, 2.04 mmol). The reaction mixture was stirred at reflux for 16 h under a Dean-Stark set-up. The reaction mass was distilled under reduced pressure. The resulting crude compound was dissolved in ethyl acetate (100 mL), the organic layers was washed with water (3×30 mL), dried over Na₂SO₄, concentrated under reduced pressure to get 4-(5,5-dimethyl-1,3-dioxan-2-yl)phenol (6 g, yield: 70.42%, LC/MS: 62%) as a brown solid. [C₁₂H₁₆O₃]; ¹H NMR (400 MHz, CDCl₃) δ 7.36 (d, J=2.0 Hz, 2H), 6.76 (d, J=2.0 Hz, 2H), 5.33 (s, 1H), 5.15 (s, 1H), 3.75 (d, J=11.2 Hz, 2H), 3.63 (d, J=10.4 Hz, 2H), 1.29 (s, 3H), 0.79 (s, 3H).

### Step 2

### 2-(4-(Methoxymethoxy)phenyl)-5,5-dimethyl-1,3-dioxane (38)

To a stirred solution of 4-(5,5-dimethyl-1,3-dioxan-2-yl)phenol (5 g, 24.03 mmol) in CH₂Cl₂ (50 mL) at room temperature was added DIPEA (15.5 g, 120.15 mmol) drop-wise. The reaction mass was cooled to 0° C. then added MOM Chloride (2.88 g, 36.05 mmol). The reaction mixture was allowed to attain room temperature and stirred for 2 h. The reaction mixture was quenched with water (30 mL) and extracted with CH₂Cl₂ (2×30 mL). The combined organic layer was dried over Na₂SO₄ and concentrated under reduced pressure to give the crude product, which was purified on a silica gel column (eluting with ethyl acetate in pet ether=7:93%) to get 2-(4-(methoxymethoxy)phenyl)-5,5-dimethyl-1,3-dioxane (2 g, yield: 33.3%, LC-MS: 81.71%) as a colourless liquid. [C₁₄H₂₀O₄]; ¹H NMR (500 MHz, CDCl₃) δ 7.43 (d, J=8.5 Hz, 2H), 7.03 (d, J=1.5 Hz, 2H), 5.34 (s, 1H), 5.16 (s, 2H), 3.75 (d, J=11.0 Hz, 2H), 3.63 (d, J=11.0 Hz, 2H), 3.45 (s, 3H), 1.28 (s, 3H), 0.79 (s, 3H).

### Step 3

### 2-(4-(Methoxymethoxy)-3-(methylthio)phenyl)-5,5-dimethyl-1,3-dioxane (39)

To a stirred solution of 2-(4-(methoxymethoxy)phenyl)-5,5-dimethyl-1,3-dioxane (1.5 g, 5.95 mmol) in THF (15 mL) at −78° C. was added n-BuLi (9 ml, 8.9 mmol, 1.5 eq). The reaction mixture was slowly allowed to attain room temperature and stirred for 20 minutes; the reaction mixture was again cooled to −78° C. and was added dimethyl disulfide (2 ml, 17.85 mmol). The reaction mixture was slowly allowed to attain room temperature and stirred for 3 h. The reaction mixture was quenched with water (20 ml) and extracted with ethyl acetate (2×25 mL). The combined organic layer was washed with brine, dried over Na₂SO₄, and concentrated under reduced pressure to get 2-(4-(methoxymethoxy)-3-(methylthio)phenyl)-5,5-dimethyl-1,3-dioxane (1.5 g, yield: 100% LC-MS: 91.59%) as a colourless liquid. [C₁₅H₂₂O₄S]; ¹H NMR (500 MHz, CDCl₃) δ 7.31 (d, J=2.0 Hz, 1H), 7.26-7.22 (m, 1H), 7.06 (d, J=8.0 Hz, 1H), 5.34 (s, 1H), 5.23 (s, 2H), 3.75 (dd, J=9.75 Hz, J=2.5 Hz, 2H), 3.63 (dd, J=11.0 Hz, J=0.5 Hz, 2H), 3.48 (s, 3H), 2.44 (s, 3H), 1.27 (s, 3H), 0.79 (s, 3H).

### Step 4

### 4-Hydroxy-3-(methylthio)benzaldehyde (40)

To a stirred solution of 2-(4-(methoxymethoxy)-3-(methylthio)phenyl)-5,5-dimethyl-1,3-dioxane (1.5 g, 5.03 mmol) in acetone (15 mL) at room temperature was added IM HCl (10 mL). The reaction mixture was maintained at 60° C. for 2 h. The reaction mixture was concentrated under reduced pressure. The crude compound was dissolved in EtOAc (30 mL), the organic layer was washed with water (2×15 mL) and dried over anhydrous Na₂SO₄, and concentrated under reduced pressure to get 4-hydroxy-3-(methylthio)benzaldehyde (1.14 g, yield: 100%, LC-MS: 81.15%) as an off white solid. [C₈H₈O₂S]; ¹H NMR (500 MHz, CDCl₃) δ 9.85 (s, 1H), 8.04 (d, J=2.0 Hz, 1H), 7.79 (dd, J=8.5 Hz, J=2.0 Hz, 1H), 7.26 (bs, 1H), 7.11 (d, J=8.5 Hz, 1H), 2.39 (s, 3H). The ¹H NMR data complies with the values reported in the literature. See Bohman, B. et al. Ang. Chem. Int. Ed. 2017, 56, 8455.

### Step 5

### 4,4′-(((2,2′-Dimethyl-[1,1′-biphenyl]-3,3′-diyl)bis(methylene))bis(oxy))bis(3-(methylthio)benzaldehyde) (41)

To a stirred solution of 3,3′-bis(bromomethyl)-2,2′-dimethyl-1,1′-biphenyl (1 g, 2.73 mmol) in ACN (10 mL) at room temperature were added 4-hydroxy-3-(methylthio)benzaldehyde (1.14 g, 6.83 mmol) and K₂CO₃ (1.5 g, 10.92 mmol) and reaction mixture was stirred at 90° C. for 4 h. The reaction mixture was cooled to room temperature, diluted with cold water (50 mL) and extracted with EtOAc (2×50 mL). The combined organic layer was washed with brine (2×50 mL), dried over anhydrous Na₂SO₄, filtered and concentrated under reduced pressure to afford 4,4′-(((2,2′-dimethyl-[1,1′-biphenyl]-3,3′-diyl)bis(methylene))bis(oxy))bis(3-(methylthio)benzaldehyde) (0.9 g, yield: 60.81%, LC/MS: 76.12%) as an off white solid. [C₃₂H₃₀O₄S₂]; ¹H NMR (500 MHz, CDCl₃) δ 9.88 (s, 2H), 7.69 (d, J=2.0 Hz, 2H), 7.65 (dd, J=8.0 Hz, J=2.0 Hz, 2H), 7.52 (d, J=7.0 Hz, 2H), 7.29 (t, J=6.0 Hz, 2H), 7.16 (d, J=6.5 Hz, 2H), 7.05 (d, J=8.5 Hz, 2H), 5.29-5.24 (m, 4H), 2.49 (s, 6H), 2.08 (s, 6H).

### Step 6

### (2S,2′S)-1,1′-(((((2,2′-dimethyl-[1,1′-biphenyl]-3,3′-diyl)bis(methylene))bis(oxy))bis(3-(methylthio)-4,1-phenylene))bis(methylene))bis(piperidine-2-carboxylic acid) di-trifluoroacetic acid salt

To a solution of 4,4′-(((2,2′-dimethyl-[1,1′-biphenyl]-3,3′-diyl)bis(methylene))bis(oxy))bis(3-(methylthio)benzaldehyde) (0.5 g, 0.92 mmol) in MeOH (20 mL) were added (S)-piperidine-2-carboxylic acid (0.595 g, 4.61 mmol) and AcOH (1 mL) and stirred at 80° C. for 4-5 h. The reaction mixture was cooled to room temperature, prior to the addition of NaCNBH₃ (0.286 g, 4.61 mmol) and stirred at 80° C. for 3 h. The reaction mass was poured into ice-cold water, basified with sat. NaHCO₃ and solid thus precipitated was filtered and dried under vacuum to get crude compound. The crude neutral product was purified by prep-HPLC under acidic conditions (Mobile phase: 0.05% Formic Acid In H₂O:ACN); Column: INERTSIL-ODS (250*20), 5u; Gradient: (T % B): 0/35, 8/60, 8.1/98, 11/98, 11.1/35, 14/35.; Flow Rate: 20 mL/min; Diluent: ACN+H₂O+THF+MeOH) to get (2S,2′S)-1,1′-(4,4′-(2,2′-dimethylbiphenyl-3,3′-diyl)bis(methylene)bis(oxy)bis(3-(methylthio)-4,1-phenylene))bis(methylene)dipiperidine-2-carboxylic acid as the di-trifluoroacetic acid salt (130 mg, yield: 18.57%, HPLC 98.38%) as a white solid. ES−, m/z 767.4 [M−H]; [C₄₄H₅₂N₂O₆S₂]; ¹H NMR (500 MHz, DMSO-d₆) δ 7.54 (d, J=7.0 Hz, 2H), 7.31 (t, J=7.75 Hz, 2H), 7.24 (bs, 6H), 7.11 (d, J=6.5 Hz, 2H), 5.28-5.23 (m, 4H), 4.41 (d, J=13.0 Hz, 2H), 4.15 (m, 2H), 3.87 (m, 2H), 3.38 (m, 2H), 2.92 (m, 2H), 2.42 (s, 6H), 2.16 (m, 2H), 2.01 (s, 6H), 1.74-1.51 (m, 10H). m.p.=134-138° C.

### HPLC Conditions:

Column: XBRIDE C18 (150 mm×4.6 mm, 3.5 μm)
Mobile phase: 10 mM Ammonium Acetate in H₂O, B: 100% Acetonitrile
Flow rate: 1.0 mL/min
T/% B (min): 0/5, 1.2/5, 3.0/55, 5.0/70, 7/95, 10/95, 12/100, 14.0/5, 16/5.

### Diluent: ACN:H₂O

### Temp.: 25° C.

Ret. time: 5.32 min

### Purity: 98.38%.

### Prep-HPLC Conditions:

### Column: INERTSIL-ODS (250*20), 5u

Mobile phase: 0.05% Formic Acid in H₂O: acetonitrile
Flow rate: 20 mL/min
T/% B (min): 0/35, 8/60, 8.1/98, 11/98, 11.1/35, 14/35.

### Diluent: ACN+H₂O+THF+MeOH

### Temp.: 25° C.

### Example 29

### (2S,2′S)-1,1′-(4,4′-(2,2′-dimethylbiphenyl-3,3′-diyl)bis(methylene)bis(oxy)bis(3-(methylthio)-4,1-phenylene))bis(methylene)dipiperidine-2-carboxylic acid di-trifluoroacetic acid salt (Cpd 29)

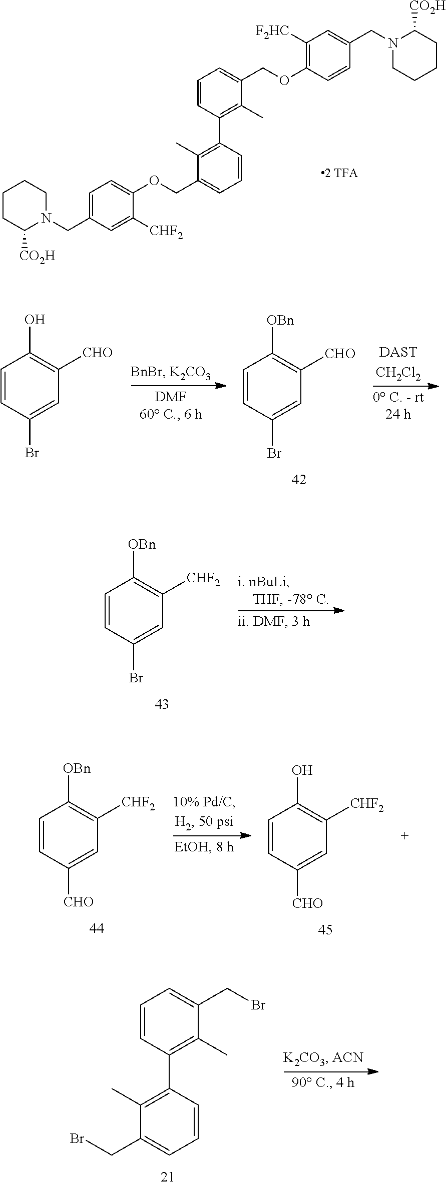

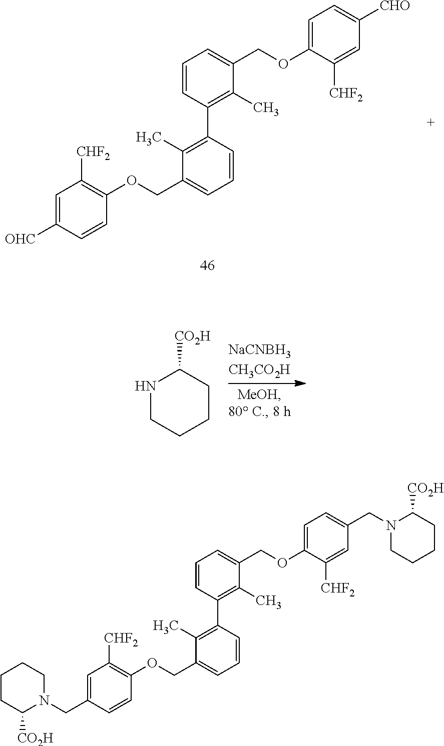 [SMILES: Cc1c(COc2ccc(C=O)cc2C(F)F)cccc1-c1cccc(COc2ccc(C=O)cc2C(F)F)c1C ; O=C(O)[C@@H]1CCCCN1 ; Cc1c(COc2ccc(CN3CCCC[C@H]3C(=O)O)cc2C(F)F)cccc1-c1cccc(COc2ccc(CN3CCCC[C@H]3C(=O)O)cc2C(F)F)c1C]

### Step 1

### 2-(Benzyloxy)-5-bromobenzaldehyde (42)

To a solution of 5-bromo-2-hydroxybenzaldehyde (6 g, 29.84 mmol) in DMF (50 mL) were added K₂CO₃ (8.2 g, 59.69 mmol) and benzyl bromide (5.61 g, 32.832 mmol) and the reaction mixture was stirred at 60° C. for 6 h. The reaction mixture was poured into ice-cold water and extracted with ethyl acetate (2×200 mL). The combined organic layer was dried over Na₂SO₄ and concentrated to give the crude product, which was purified on a silica gel column, eluting with ethyl acetate in pet ether (0 to 20%) to get 2-(benzyloxy)-5-bromobenzaldehyde (8.5 g, 98%) as a white solid. ES+, m/z 296 [M+H]; [C₁₄H₁₁BrO₂]; ¹H NMR (500 MHz, CDCl₃): δ 10.45 (s, 1H), 7.94 (d, J=2.5 Hz, 1H), 7.61-7.58 (m, 1H), 7.42-7.38 (m, 5H), 6.95 (d, J=8.5 Hz, 1H), 5.18 (s, 2H).

### Step 2

### 1-(Benzyloxy)-4-bromo-2-(difluoromethyl)benzene (43)

A solution of DAST (3.23 g, 20.10 mmol) in CH₂Cl₂ (30 mL) was added drop wise to a solution of 2-(benzyloxy)-5-bromobenzaldehyde (4.5 g, 15.46 mmol) in CH₂Cl₂ (20 mL) cooled to 0° C. Then after the reaction mixture was stirred at room temperature for 24 h. The reaction was quenched by drop-wise addition of saturated NH₄Cl solution and diluted with DCM (100 mL); the resulting organic layer was washed with water (50 mL), brine (50 mL), dried over Na₂SO₄, and concentrated to give a crude product, which was purified on a silica gel column chromatography (eluting with pet-ether: ethyl acetate=90:10) to afford 1-(benzyloxy)-4-bromo-2-(difluoromethyl)benzene as a white solid. (2.7 g, 55%, LC/MS, 88.5%) [C₁₄H₁₁BrF₂O]; ¹H NMR (400 MHz, CDCl₃) δ 7.69-7.68 (m, 1H), 7.50-7.47 (m, 1H), 7.39-7.33 (m, 5H), 6.94 (t, J=69 Hz, 1H), 6.85 (d, J=11.0 Hz, 1H), 5.11 (s, 2H).

### Step 3

### 4-(Benzyloxy)-3-(difluoromethyl)benzaldehyde (44)

To a cooled solution of 1-(benzyloxy)-4-bromo-2-(difluoromethyl)benzene (2.7 g, 8.681 mmol) in dry THF (50 mL) at −78° C. was added n-BuLi (6.5 mL, 10.41 mmol) drop-wise. The resulting solution was stirred at same temperature for 15 min prior to the drop-wise addition of DMF (1.3 mL, 42.28 mmol) at −78° C. for 30 min. The reaction mixture was stirred at same temperature for 2 h and quenched with saturated NH₄Cl solution (20 mL). The resulting solution was diluted with water, extracted into ethyl acetate. The organic layer was washed with water, brine, dried over Na₂SO₄, filtered concentrated under reduced pressure. The residue was purified by column chromatography (eluting with pet-ether:ethyl acetate=80:20) to afford 4-(benzyloxy)-3-(difluoromethyl)benzaldehyde (2.4 g, LC/MS, 62%) as an off white solid. ES+, m/z 263 [M+H]; [C₁₅H₁₂F₂O₂]; ¹H NMR (500 MHz, CDCl₃): δ 9.95 (s, 1H), 8.25 (s, 1H), 7.95-7.94 (m, 1H), 7.43-7.33 (m, 5H), 7.12 (d, J=8.5 Hz, 1H), 7.01 (t, J=33.0 Hz, 1H), 6.88 (d, J=7.5 Hz, 1H), 5.24 (s, 2H).

### Step 4

### 3-(Difluoromethyl)-4-hydroxybenzaldehyde (45)

To a solution of 4-(benzyloxy)-3-(difluoromethyl)benzaldehyde (2.4 g, 9.16 mmol) in ethanol (50 mL) was added 10% Pd/C (0.3 g) in a Parr shaker bottle. The reaction mixture was shaken at room temperature for 8 h under hydrogen (50 psi). The reaction mixture was filtered through celite pad and the filtrate was evaporated to give a crude product, which was purified by GRACE flash chromatography, (eluting with pet-ether:ethyl acetate (20:80) to afford 3-(difluoromethyl)-4-hydroxybenzaldehyde (900 mg, 57%, LC/MS; 93.6%) as a yellow solid. ES+, m/z 172.9 [M+H]; [C₈H₆F₂O₂]; ¹H NMR (500 MHz, DMSO-d₆); δ 11.45 (s, 1H), 9.86 (s, 1H), 8.0 (s, 1H), 7.92-7.89 (m, 1H), 7.11 (t, J=53.0 Hz, 1H).

### Step 5

### 4,4′-(2,2′-Dimethylbiphenyl-3,3′-diyl)bis(methylene)bis(oxy)bis(3-(difluoromethyl) benzaldehyde) (46)

To a stirred solution of 3,3′-bis(bromomethyl)-2,2′-dimethyl-1,1′-biphenyl (450 mg, 1.22 mmol) in ACN (10 mL) at room temperature were added 3-(difluoromethyl)-4-hydroxybenzaldehyde (690 mg, 4.032 mmol) and K₂CO₃ (674 mg, 4.88 mmol) and reaction mixture was stirred at 90° C. for 4 h. The reaction mixture was cooled to room temperature, diluted with cold water (50 mL) and extracted with EtOAc (2×50 mL). The combined organic layer was washed with brine (2×50 mL), dried over anhydrous Na₂SO₄, filtered and concentrated under reduced pressure to afford 4,4′-(2,2′-dimethylbiphenyl-3,3′-diyl)bis(methylene)bis(oxy)bis(3-(difluoromethyl) benzaldehyde) (0.6 g, yield: 44%, LC/MS; 85%) as a white solid. ES+, m/z 551.2 [M+H]; [C₃₂H₂₆F₄O₂]; ¹H NMR (400 MHz, CDCl₃): δ 9.95 (s, 2H), 8.14 (s, 2H), 8.03-8.00 (m, 2H), 7.43 (d, J=6.8 Hz, 2H), 7.30 (t, J=7.6 Hz, 2H), 7.22-7.18 (m, 4H), 6.97 (t, J=69 Hz, 2H), 5.22 (s, 4H), 2.05 (s, 6H).

### Step 6

### (2S,2′S)-1,1′-(((((2,2′-Dimethyl-[1,1′-biphenyl]-3,3′-diyl)bis(methylene))bis(oxy))bis(3-(difluoromethyl)-4,1-phenylene))bis(methylene))bis(piperidine-2-carboxylic acid) di-trifluoroacetic acid salt

To a solution of 4,4′-(2,2′-dimethylbiphenyl-3,3′-diyl)bis(methylene)bis(oxy)bis(3-(difluoromethyl)benzaldehyde) (600 mg, 1.090 mmol) in MeOH (20 mL) were added (S)-piperidine-2-carboxylic acid (704 mg, 5.454 mmol) and AcOH (0.5 mL) and stirred at 80° C. for 5 h. The reaction mixture was cooled to room temperature, prior to the addition of NaCNBH₃ (338 mg, 5.454 mmol) and stirred at 80° C. for 3 h. The reaction mass was poured into ice-cold water, basified with sat. NaHCO₃ and solid thus precipitated was filtered and dried under vacuum to get crude compound. The crude neutral product was purified by prep-HPLC under acidic conditions (Mobile phase: 0.1% TFA in H₂O:ACN; Column: INERTSIL ODS-C18 (19*120 MM), 5u; Gradient: (T % B): 0/23, 8/50, 11/50, 11.1/98, 13/98, 13.1/23, 16/23; Flow Rate: 20 mL/min; Diluent: ACN+H₂O+THF+MeOH) to get (2S,2′S)-1,1′-(((((2,2′-dimethyl-[1,1′-biphenyl]-3,3′-diyl)bis(methylene))bis(oxy))bis(3-(difluoromethyl)-4,1-phenylene)) bis(methylene)) bis(piperidine-2-carboxylic acid) as the di-trifluoroacetic acid salt (200 mg, yield: 23%, HPLC 97.7%) as an off white solid. ES+, m/z 775.3 [M−H]; [C₄₄H₄₈F₄N₂O₆] ¹H NMR (500 MHz, DMSO-d₆): δ 9.91 (s, 1H), 7.65 (s, 2H), 7.61 (d, J=8.0 Hz, 2H), 7.52 (d, J=7.0 Hz, 2H), 7.44 (d, J=8.5 Hz, 2H), 7.31 (t, J=7.5 Hz, 2H), 7.15 (t, J=55.0 Hz, 2H), 7.12 (d, J=7.0 Hz, 2H), 5.30 (s, 4H), 4.44 (d, J=13.0 Hz, 2H), 4.13 (m, 2H), 3.92 (m, 2H), 3.23 (m, 2H), 2.92 (m, 2H), 2.14 (m, 2H), 2.01 (s, 6H), 1.72-1.68 (m, 8H), 1.52 (m, 2H). m.p=142-146° C.

### HPLC Conditions:

Column: XBRIDE C18 (150 mm×4.6 mm, 3.5 μm)
Mobile phase: 10 mM Ammonium Acetate in H₂O, B: 100% Acetonitrile
Flow rate: 1.0 mL/min
T/% B (min): 0/5, 1.2/5, 3.0/55, 5.0/70, 7/95, 10/95, 12/100, 14.0/5, 16/5.

### Diluent: ACN:H₂O

### Temp.: 25° C.

Ret. time: 5.32 min

### Purity: 97.2%.

### PreP-HPLC Conditions:

### Column: INERTSIL ODS-C18 (19*120 MM), 5u

Mobile phase: 0.1% TFA in H₂O: ACN
Flow rate: 25 mL/min
T/% B (min): 0/23, 8/50, 11/50, 11.1/98, 13/98, 13.1/23, 16/23.

### Diluent: ACN+H₂O+THF+MeOH

### Temp.: 25° C.

### Example 30

### 2,2′,2″,2′″-((((((2,2′-Dimethyl-[1,1′-biphenyl]-3,3′-diyl)bis(methylene))bis(oxy))bis(3-bromo-4,1-phenylene))bis(methylene))bis(azanetriyl))tetraacetic acid di-trifluoroacetic acid salt (Cpd 30)

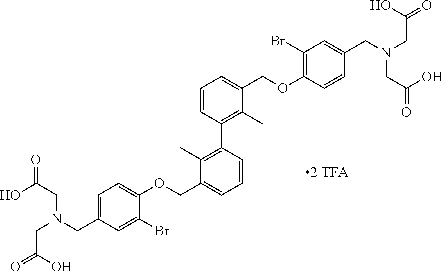 [SMILES: Cc1c(COc2ccc(CN(CC(=O)O)CC(=O)O)cc2Br)cccc1-c1cccc(COc2ccc(CN(CC(=O)O)CC(=O)O)cc2Br)c1C]

To a solution of 4,4′-(2,2′-dimethylbiphenyl-3,3′-diyl)bis(methylene)bis(oxy)bis(3-bromobenzaldehyde) 5 (300 mg, 0.495 mmol) in MeOH (20 mL) were added 2,2′-azanediyldiacetic acid (329 mg, 2.47 mmol) and cat. AcOH (3-4 drops) and stirred at 80° C. for 4-5 h. The reaction mixture was cooled to room temperature, prior to the addition of NaCNBH₃ (155 mg, 2.47 mmol). The reaction mixture was stirred at 80° C. for 4 h. The reaction mixture was poured into ice cold water and solid thus precipitated was filtered and dried under vacuum to get crude compound. The crude neutral product was purified by prep-HPLC under acidic conditions (Mobile phase: 0.1% trifluoroacetic acid in H₂O:ACN Column: KROMOSIL-C18 (150*25 MM), 10u GRADIENT: (T % B): 0/20, 8/53, 9/53, 9.1/98, 10/98, 10.1/20, 12/20 Flow Rate: 25 mL/min Diluent: ACN+H₂O+THF+MEOH) to give 2,2′,2″,2′″-((((((2,2′-dimethyl-[1,1′-biphenyl]-3,3′-diyl)bis(methylene))bis(oxy))bis(3-bromo-4,1-phenylene))bis(methylene))bis(azanetriyl)) tetraacetic acid as the di-trifluoroactic acid salt (31 mg, yield: 12.7%, HPLC 98.36%) as a white solid. ES−, m/z 838.6 [M−H]; [C₃₈H₃₈Br₂N₂O₁₀]; ¹H NMR (500 MHz, DMSO-d₆) δ 12.28 (bs, 2H), 7.60 (d, J=1.5 Hz, 2H), 7.54 (d, J=7.5 Hz, 2H), 7.32-7.29 (m, 4H), 7.25 (d, J=8.5 Hz, 2H), 7.10 (d, J=7.0 Hz, 2H), 5.23 (s, 4H), 3.75 (s, 4H), 3.39 (s, 8H), 2.03 (s, 6H). m.p.=223-227° C.

### HPLC Conditions:

Column: Acquity UPLC BEH C18 (100 mm×2.1 mm, 1.7 μm)
Mobile phase: 0.025% TFA in ACN
Flow rate: 0.4 mL/min
T/% B (min): 0/10, 1.5/10, 8/98, 12/98, 12.01/10

### Diluent: (ACN-WATER 70:30)

### Temp.: Ambient

Ret. time: 5.60 min

### Purity: 98.36%.

### Prep-HPLC Conditions:

Mobile phase: 0.1% TFA in H₂O:ACN

### Column: KROMOSIL-C18 (150*25 MM), 10u

### GRADIENT: (T % B): 0/20, 8/53, 9/53, 9.1/98, 10/98, 10.1/20, 12/20

Flow Rate: 25 mL/min

### Diluent: ACN+H₂O+THF+MeOH

### Example 31

### 2,2′,2″,2′″-((((((2,2′-Dimethyl-[1,1′-biphenyl]-3,3′-diyl)bis(methylene))bis(oxy))bis(3-bromo-4,1-phenylene))bis(methylene))bis(azanetriyl))tetrakis(ethan-1-ol) di-trifluoroacetic acid salt (Cpd 31)

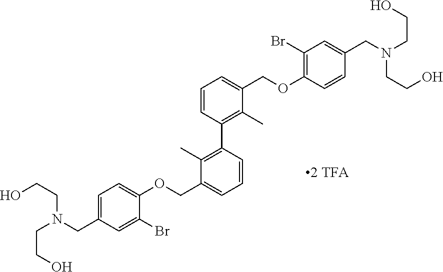 [SMILES: Cc1c(COc2ccc(CN(CCO)CCO)cc2Br)cccc1-c1cccc(COc2ccc(CN(CCO)CCO)cc2Br)c1C]

To a solution of 4,4′-(((2,2′-dimethyl-[1,1′-biphenyl]-3,3′-diyl)bis(methylene))bis(oxy))bis(3-bromobenzaldehyde) 5 (250 mg, 0.41 mmol) in MeOH (20 mL) were added 2,2′-azanediylbis(ethan-1-ol) (216.5 mg, 2.06 mmol) and AcOH (3-4 drops) and stirred at 80° C. for 4-5 h. The reaction mixture was cooled to room temperature, prior to the addition of NaCNBH₃ (129.53 mg, 2.06 mmol). The reaction mixture was stirred at 80° C. for 4 h. The reaction mixture was poured into ice cold water and solid thus precipitated was filtered and dried under vacuum to get crude compound. The crude neutral product was purified by prep-HPLC under acidic conditions (Mobile phase: 0.1% TFA in H₂O:ACN; Column: YMC-TRIART C18 (150*25 MM), 10u; Gradient (% B): 0/40, 10/40, 10.1/98, 12/98, 12.1/40, 14/40; Flow Rate: 25 mL/min; Diluent: ACN+MeOH+H₂O) to give 2,2′,2″,2′″-((((((2,2′-dimethyl-[1,1′-biphenyl]-3,3′-diyl)bis(methylene))bis(oxy))bis(3-bromo-4,1-phenylene))bis(methylene))bis(azanetriyl)) tetrakis(ethan-1-ol) (110 mg, as the di-TFA salt, yield: 34%, UPLC 98.05%) as a white solid. ES+, m/z 785.2 [M+H]; [C₃₈H₄₆Br₂N₂O₆]; ¹H NMR (500 MHz, DMSO-d₆) δ 9.35 (s, 2H), 7.87 (s, 2H), 7.56-7.55 (m, 4H), 7.40 (d, J=7.0 Hz, 2H), 7.32 (t, J=8.0 Hz, 2H), 7.12 (d, J=7.0 Hz, 2H), 5.33-5.29 (m, 6H), 4.38 (m, 4H), 3.77 (m, 8H), 3.32-3.15 (m, 8H), 2.04 (s, 6H). m.p.=118-122° C.

### UPLC Method Conditions:

Column: Acquity UPLC BEH C18 (100 mm×2.1 mm, 1.7 μm)
Mobile phase-A: 0.025% TFA in ACN; B: 0.025% TFA in Water

### Gradient Time % A: 0/10, 1.5/10, 8/98, 12/98, 12.01/10

Flow rate: 0.4 mL/min

### Column Temp.: Ambient

Diluent: (ACN:water 70:30)
Retention time: 5.43 min

### Purity: 97.95%.

### HPLC Method Conditions

Mobile phase: 0.1% formic acid in H₂O:ACN
Column: YMC-TRIART C18 (150*25 mm), 10u

### GRADIENT: (T % B): 0/40, 10/40, 10.1/98, 12/98, 12.1/40, 14/40

Flow Rate: 25 mL/min

### Example 32

### 2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(propane-1,3-diol) di-trifluoroacetic acid salt (Cpd 32)

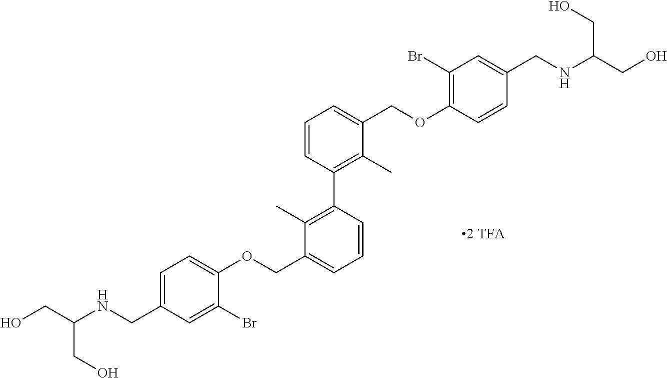 [SMILES: Cc1c(COc2ccc(CNC(CO)CO)cc2Br)cccc1-c1cccc(COc2ccc(CNC(CO)CO)cc2Br)c1C]

To a solution of 4,4′-(((2,2′-dimethyl-[1,1′-biphenyl]-3,3′-diyl)bis(methylene))bis(oxy))bis(3-bromobenzaldehyde) 5 (250 mg, 0.41 mmol) in MeOH (20 mL) were added 2-aminopropane-1,3-diol (187.6 mg, 2.06 mmol) and AcOH (0.5 mL) and stirred at 80° C. for 5 h. The reaction mixture was cooled to room temperature, prior to the addition of NaCNBH₃ (129.53 mg, 2.06 mmol). The reaction mixture was stirred at 80° C. for 4 h. The reaction mixture was poured into ice cold water and solid thus precipitated was filtered and dried under vacuum to get crude compound. The crude neutral product was purified by prep-HPLC under acidic conditions (Mobile phase: 0.1% TFA in H₂O: MeOH; Column: SYMMETRY-C8(300*19 MM), 5u; Gradient (% B): 0/55, 8/75, 9/75, 9.1/98, 11/98, 11.1/55, 14/55; Flow Rate: 25 mL/min; Diluent: ACN+MeOH+H₂O+THF) to get 2,2′-((((((2,2′-dimethyl-[1,1′-biphenyl]-3,3′-diyl)bis(methylene))bis(oxy))bis(3-bromo-4,1-phenylene))bis(methylene))bis(azanediyl))bis(propane-1,3-diol) (180 mg, as the di-TFA salt, yield: 12.7%, UPLC 98.62%) as a white solid. ES+, m/z 757.4 [M+H]; [C₃₆H₄₂Br₂N₂O₆]; ¹H NMR (500 MHz, DMSO-d₆) δ 8.70 (bs, 4H), 7.82 (d, J=2.0 Hz, 2H), 7.55-7.50 (m, 4H), 7.37 (d, J=8.5 Hz, 2H), 7.31 (t, J=8.0 Hz, 2H), 7.11 (d, J=7.5 Hz, 2H), 5.28 (bs, 8H), 4.17 (m, 4H), 3.68-3.64 (m, 8H), 3.06 (m, 2H), 2.03 (s, 6H). m.p.=148-152° C.

### UPLC Method Conditions:

Column: Acquity UPLC BEH C18 (100 mm×2.1, 1.7 μm)

### Mobilephase-A: 0.025% TFA in ACN; B: 0.025% TFA in Water

### Gradient Time % A: 0/10, 1.5/10, 8/98, 12/98, 12.01/10

Flow rate: 0.4 mL/min

### Column Temp.: Ambient

### Diluent: (ACN-WATER 70:30)

Retention time: 5.32 min

### Purity: 98.6%.

HPLC method Conditions:
Mobile phase: 0.1% TFA in H₂O:MeOH

### Column: SYMMETRY-C8(300*19 MM), 5u

### Gradient: (T % B): 0/55, 8/75, 9/75, 9.1/98, 11/98, 11.1/55, 14/55

Flow Rate: 25 mL/min

### Diluent: ACN+H₂O+THF

### Example 33

### 2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-methylpropane-1,3-diol) di-trifluoroacetic acid salt (Cpd 33)

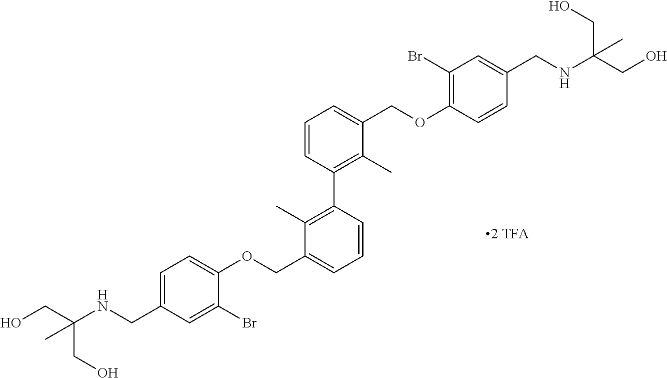 [SMILES: Cc1c(COc2ccc(CNC(C)(CO)CO)cc2Br)cccc1-c1cccc(COc2ccc(CNC(C)(CO)CO)cc2Br)c1C]

To a solution of 4,4′-(((2,2′-dimethyl-[1,1′-biphenyl]-3,3′-diyl)bis(methylene))bis(oxy))bis(3-bromobenzaldehyde) 5 (250 mg, 0.41 mmol) in MeOH (25 mL) were added 2-amino-2-methylpropane-1,3-diol (215.8 mg, 2.05 mmol) and cat. AcOH (3-4 drops) and stirred at 80° C. for 4-5 h. The reaction mixture was cooled to room temperature, prior to the addition of NaCNBH₃ (259.2 mg, 4.125 mmol). The reaction mixture was stirred at 80° C. for 4 h. The reaction mixture was poured in to ice cold water and solid thus precipitated was filtered and dried under vacuum to get crude compound. The crude neutral product was purified by prep-HPLC two times under acidic conditions (Mobile phase: 0.1% TFA in H₂O:ACN; Column: Kromasil C18 (150*25 MM), 10u; Gradient (% B): 0/20, 8/40, 10/40, 10.1/98, 13/98, 13.1/20, 16/20; Flow Rate: 25 mL/min; Diluent: ACN+MeOH+H₂O+1% TFA) to obtain 2,2′-((((((2,2′-dimethyl-[1,1′-biphenyl]-3,3′-diyl)bis(methylene))bis(oxy))bis(3-bromo-4,1-phenylene))bis(methylene))bis(azanediyl))bis(2-methylpropane-1,3-diol) (120 mg, as the di-TFA salt, yield: 12.7%, HPLC 97.95%) as a white solid. ES+, m/z 785.4 [M+H]; [C₃₈H₄₆Br₂N₂O₆]; ¹H NMR (500 MHz, DMSO-d₆) δ 8.45 (bs, 4H),7.78 (d, J=2.0 Hz, 2H), 7.54 (d, J=7.0 Hz, 2H), 7.48 (d, J=2.0 Hz, 2H), 7.47 (d, J=2.0 Hz, 2H), 7.31 (t, J=7.5 Hz, 2H), 7.10 (d, J=7.5 Hz, 2H), 5.45 (t, J=4.5 Hz, 4H), 5.29 (s, 4H), 4.08-4.07 (m, 4H), 3.60-3.53 (m, 8H), 2.03 (s, 6H), 1.21 (s, 6H). m.p.=134-138° C.

### UPLC Method Conditions:

Column: Acquity UPLC BEH C18 (100 mm×2.1 mm, 1.7 μm)
Mobile phase-A: 0.025% TFA in ACN; B: 0.025% TFA in Water

### Gradient Time % A: 0/10, 1.5/10, 8/98, 12/98, 12.01/10

Flow rate: 0.4 mL/min

### Column Temp.: Ambient

### Diluent: (ACN-WATER 70:30)

Retention time: 5.43 min

### Purity: 97.95%

### Prep-HPLC Method Conditions:

Mobile phase: 0.1% TFA ACID in H₂O:acetonitrile
Column: Kromasil C18 (150*25 mm), 10u

### Gradient (T % B): 0/20, 8/40, 10/40, 10.1/98, 13/98, 13.1/20, 16/20

Flow Rate: 25 mL/min

### Diluent: ACN+H₂O+THF

### Example 34

### (2S,2′S)-1,1′-(((((2,2′-dimethyl-[1,1′-biphenyl]-3,3′-diyl)bis(methylene))bis(oxy))bis(3-cyano-4,1-phenylene))bis(methylene))bis(piperidine-2-carboxylic acid) di-trifluoroacetic acid salt (Cpd 34)

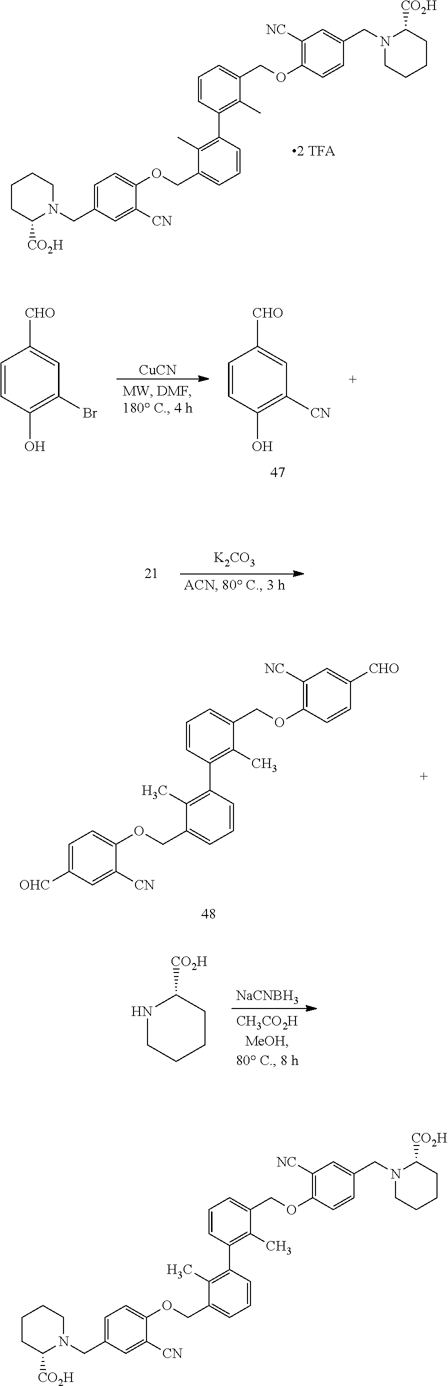

### Step 1

### 5-Formyl-2-hydroxybenzonitrile (47)

To a stirred solution of 3-bromo-4-hydroxybenzaldehyde (5 g, 24.87 mmol) in DMF (50 mL) at room temperature was added CuCN (3.3 g, 37.31 mmol). The reaction mass was heated to 180° C. under microwave for 4 h. The reaction mass was filtered through celite pad, the filtrate was concentrated under reduced pressure to get crude compound. The crude compound was purified by silica gel column chromatography (100-200) using ethyl acetate in pet ether (30:70) and obtained 5-formyl-2-hydroxybenzonitrile (0.25 g, yield: 6.84% LC-MS: 89.45%) as a brown solid. [C₈H₅NO₂] ¹H NMR (400 MHz, DMSO-d₆); δ 12.15 (bs, 1H), 9.82 (s, 1H), 8.23 (d, J=2.4 Hz, 1H), 8.02-7.99 (m, 1H), 7.17 (d, J=8.4 Hz, 1H).

### Step 2

### 6,6′-(((2,2′-Dimethyl-[1,1′-biphenyl]-3,3′-diyl)bis(methylene))bis(oxy))bis(3-formylbenzonitrile) (48)

To a stirred solution of 3,3′-bis(bromomethyl)-2,2′-dimethyl-1,1′-biphenyl (21) (250 mg, 0.683 mmol) in ACN (10 mL) at room temperature were added K₂CO₃ (377 mg, 2.73 mmol) and 5-formyl-2-hydroxybenzonitrile (251 mg, 0.707 mmol) and stirred at 80° C. for 3 h. The reaction mixture was concentrated to remove most of the solvent. The residue was poured into ice-water and the resulting precipitate was collected by filtration and dried in vacuum to get 6,6′-(2,2′-dimethylbiphenyl-3,3′-diyl)bis(methylene)bis(oxy)bis(3-formylbenzonitrile) (270 mg, yield: 79%, LC/MS: 80.4%) an off-white solid; [C₃₂H₂₄N₂O₄] ¹H NMR (400 MHz, CDCl₃) δ 9.91 (s, 2H), 8.14-8.08 (m, 4H), 7.48 (d, J=7.2 Hz, 2H), 7.30 (t, J=7.6 Hz, 2H), 7.24 (s, 2H), 7.19 (d, J=6.4 Hz, 2H), 5.34 (s, 4H), 2.08 (s, 6H).

### Step 3

### (2S,2′S)-1,1′-(((((2,2′-Dimethyl-[1,1′-biphenyl]-3,3′-diyl)bis(methylene))bis(oxy))bis(3-cyano-4,1-phenylene))bis(methylene))bis(piperidine-2-carboxylic acid) di-trifluoroacetic acid salt

To a solution of 6,6′-(((2,2′-dimethyl-[1,1′-biphenyl]-3,3′-diyl)bis(methylene))bis(oxy))bis(3-formylbenzonitrile) (350 mg, 0.70 mmol) in MeOH (15 mL) were added (S)-piperidine-2-carboxylic acid (452 mg, 3.5 mmol) and acetic acid (0.5 mL) and the reaction mixture was stirred at 80° C. for 2 h. The reaction mixture was cooled to room temperature, prior to the addition of NaCNBH₃ (220 mg, 3.50 mmol) and then the reaction mixture was stirred at 80° C. for 6 h. The reaction mixture was poured into ice cold water, and solid thus precipitated was filtered and dried under vacuum. The crude neutral compound was purified by Prep-HPLC under acidic conditions to get (2S,2′S)-1,1′-(((((2,2′-dimethyl-[1,1′-biphenyl]-3,3′-diyl)bis(methylene))bis(oxy))bis(3-cyano-4,1-phenylene))bis(methylene))bis(piperidine-2-carboxylic acid); 200 mg (as the di-TFA Salt), yield: 39%, LC/MS ES−, m/z 725.3 [M−H]; 98.16%, HPLC: 98.72%) as an off white solid. [C₄₄H₄₆N₄O₆]; ¹H NMR (500 MHz, DMSO-d₆) δ 9.87 (bs, 1H), 7.81 (s, 2H), 7.75 (d, J=8.5 Hz, 2H), 7.54 (t, J=7.5 Hz, 4H), 7.33 (t, J=7.5 Hz, 2H), 7.14 (d, J=7.5 Hz, 2H), 5.39 (s, 4H), 4.34 (m, 2H), 4.07 (m, 2H), 3.87 (m, 2H), 3.43-3.16 (m, 2H), 2.86-2.85 (m, 2H), 2.11-2.07 (m, 2H), 2.04 (s, 6H), 1.69-1.65 (m, 8H), 1.51-1.50 (m, 2H). m.p.=196-200° C.

### UPLC Method Conditions:

Column: Acquity UPLC BEH C18 (100 mm×2.1 mm, 1.7 μm)
Mobile phase: 0.025% TFA in ACN, Mobile phase-B: 0.025% TFA in Water
Flow rate: 0.4 mL/min
T/% B (min): 0/10, 1.5/10, 8/98, 12/98, 12.01/10

### Diluent: ACN:H₂O:MeOH

### Temp.: 25° C.

Ret. time: 5.19 min

### Purity: 98.72%.

### Prep-HPLC Conditions:

### Column: INERTSIL-ODS (250*20), 5u

Mobile phase: Mobile phase: 0.1% TFA in H₂O:ACN
Flow rate: 20 mL/min
T/% B (min): 0/20, 8/50, 10.4/50, 10.5/98, 13/98, 13.1/20, 16/20

### Diluent: ACN+H₂O+THF+MeOH

### Temp.: 25° C.

### Example 35

### (2S,2′S)-1,1′-(((((2,2′-Dimethyl-[1,1′-biphenyl]-3,3′-diyl)bis(methylene))bis(oxy))bis(5-chloro-2-((5-cyanopyridin-3-yl)methoxy)-4,1-phenylene))bis(methylene))bis(piperidine-2-carboxylic acid) di-trifluoroacetic acid salt (Cpd 35)

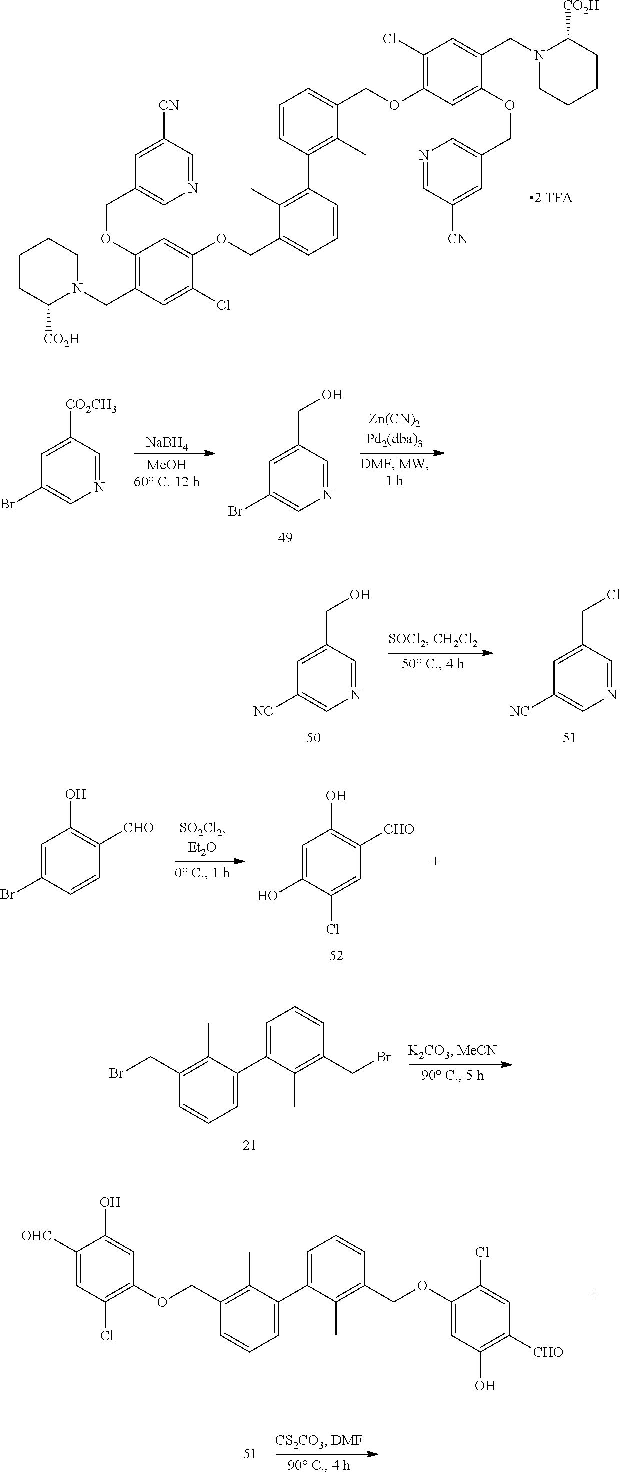

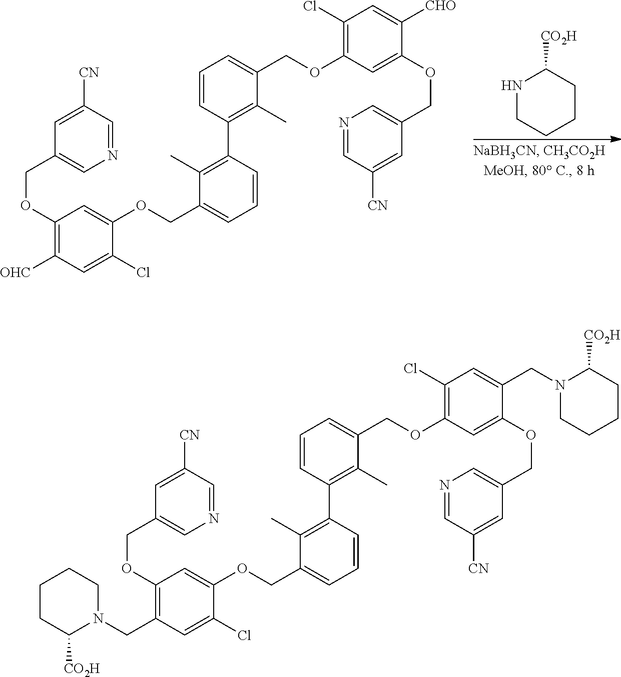 [SMILES: Cc1c(COc2cc(OCc3cncc(C#N)c3)c(C=O)cc2Cl)cccc1-c1cccc(COc2cc(OCc3cncc(C#N)c3)c(C=O)cc2Cl)c1C ; O=C(O)[C@@H]1CCCCN1 ; Cc1c(COc2cc(OCc3cncc(C#N)c3)c(CN3CCCC[C@H]3C(=O)O)cc2Cl)cccc1-c1cccc(COc2cc(OCc3cncc(C#N)c3)c(CN3CCCC[C@H]3C(=O)O)cc2Cl)c1C]

### Step 1

### (5-Bromopyridin-3-yl)methanol (49)

To a stirred solution of methyl 5-bromonicotinate (10 g, 43.47 mmol) in methanol (100 mL) NaBH₄ (6.5 g, 260.0 mmol) was added portion wise at 0° C. The reaction mixture was allowed warm to room temperature and stirred at 50° C. for 16 h. The solvents were removed by rotary evaporation. The residue was diluted with ice-cold water (100 mL) and the aqueous layer was extracted with EtOAc (3×150 mL); the combined organic layer was washed with brine (2×150 mL), dried over anhydrous Na₂SO₄, filtered and concentrated under reduced pressure to afford (5-bromopyridin-3-yl)methanol (6.5 g, yield: 74%, LC/MS: 99%) as an off-white solid. NMR (CDCl₃, 400 MHz): δ 8.58 (d, J=2.0 Hz, 1H), 8.48 (d, J=2.0 Hz, 1H), 7.90 (t, J=2.0 Hz, 1H), 4.73 (s, 2H), 2.47 (bs, 1H).

### Step 2

### 5-(Hydroxymethyl)nicotinonitrile (50)

To a stirred solution of (5-bromopyridin-3-yl)methanol (3.1 g, 16.57 mmol) in DMF (50 mL) at room temperature were added Zn(CN)₂ (2 g, 16.57 mmol), dppf (460 mg, 0.828 mmol) and Pd₂(dba)₃ (307 mg, 0.335 mmol). The reaction mass was heated to 170° C. under microwave for 4 h. The reaction mass was filtered through celite pad, the filtrate was concentrated under reduced pressure to get crude compound. The crude compound was purified by silica gel column chromatography (100-200) using CH₂Cl₂ in MeOH (90:10) as eluent and obtained 5-(hydroxymethyl)nicotinonitrile (800 mg, yield: 35% LC/MS: 88%) as a brown solid. NMR (CDCl₃, 400 MHz): δ 8.81 (dd, J=9.6 Hz, 6.0 Hz, 2H), 8.03-8.02 (m, 1H), 4.83 (s, 2H), 2.11 (bs, 1H).

### Step 3

### 5-(Chloromethyl)nicotinonitrile (51)

A solution of 5-(hydroxymethyl)nicotinonitrile (1 g, 7.462 mmol) in dry CH₂Cl₂ (15 mL) was treated with thionyl chloride (1.7 g, 14.92 mmol) and stirred at 50° C. for 3 h. The reaction mixture was concentrated under reduced pressure to afford the 5-(chloromethyl)nicotinonitrile HCl (900 mg) as a white solid. This compound was used immediately for next step.

### Step 4

### 5-Chloro-2,4-dihydroxybenzaldehyde (52)

To a solution of 2,4-dihydroxybenzaldehyde (20 g, 144.92 mmol) in diethyl ether (500 mL) was added drop wise sulfuryl chloride (14 mL, 171.2 mmol) at 0° C. under argon and stirred at room temperature for 30 min. The reaction mixture was poured into ice-water, the organic layer was separated and washed with water, brine, dried over Na₂SO₄, filtered, and concentrated to give the crude product, which was purified on a silica gel column (pet. ether:ethyl acetate=90:10) to get 5-chloro-2,4-dihydroxybenzaldehyde as an off white solid. (6.7 g, 26%, LC/MS; 97%). ES−, m/z 170.9 [M−H]; [C₇H₅ClO₃]; NMR (DMSO-d₆, 400 MHz): δ 11.39 (1H, s), 10.87 (1H, s), 9.98 (1H, s), 7.0 (1H, s), 6.58 (1H, s).

### Step 5

### 4,4′-(((2,2′-Dimethyl-[1,1′-biphenyl]-3,3′-diyl)bis(methylene))bis(oxy))bis(5-chloro-2-hydroxybenzaldehyde) (53)

To a stirred solution of 3,3′-bis(bromomethyl)-2,2′-dimethyl-1,1′-biphenyl (1 g, 2.732 mmol) in ACN (20 mL) at room temperature were added K₂CO₃ (1.5 g, 10.928 mmol) and 5-chloro-2,4-dihydroxybenzaldehyde (1.4 g, 8.196 mmol) and stirred at 80° C. for 3 h. The reaction mixture was concentrated to remove most of the solvent. The residue was poured into ice-water and the resulting precipitate was collected by filtration and dried in vacuum to get 4,4′-(((2,2′-dimethyl-[1,1′-biphenyl]-3,3′-diyl)bis(methylene))bis(oxy))bis(5-chloro-2-hydroxybenzaldehyde) (950 g, yield: 63%); [C₃₀H₂₄Cl₂O₆]; ¹H NMR (400 MHz, CDCl₃) δ 11.17 (s, 2H), 10.04 (s, 2H), 7.71 (s, 2H), 7.52 (d, J=7.2 Hz, 2H), 7.33-7.31 (m, 2H), 7.13 (d, J=7.6 Hz, 2H), 6.87 (s, 2H), 5.33 (s, 4H), 2.01 (s, 6H).

### Step 6

### 5,5′-((((((2,2′-Dimethyl-[1,1′-biphenyl]-3,3′-diyl)bis(methylene))bis(oxy))bis(4-chloro-6-formyl-3,1-phenylene))bis(oxy))bis(methylene))dinicotinonitrile) (54)

To a stirred solution of 4,4′-(((2,2′-dimethyl-[1,1′-biphenyl]-3,3′-diyl)bis(methylene))bis(oxy))bis(5-chloro-2-hydroxybenzaldehyde) (600 mg, 1.090 mmol) in DMF (10 mL) at room temperature were added Cs₂CO₃ (2.13 g, 6.545 mmol) and 5-(chloromethyl)nicotinonitrile (829 mg, 4.39 mmol) and stirred at 90° C. for 3 h. The reaction mixture was concentrated to remove most of the solvent. The residue was poured into ice-water and the resulting precipitate was collected by filtration and dried in vacuum to get 5,5′-((((((2,2′-dimethyl-[1,1′-biphenyl]-3,3′-diyl)bis(methylene))bis(oxy))bis(4-chloro-6-formyl-3,1-phenylene))bis(oxy)) bis(methylene))dinicotinonitrile (700 mg, yield: 82%, LC/MS; 74%) as a brown solid. ¹H NMR (400 MHz, DMSO-d₆) δ 10.28 (s, 2H), 8.91 (s, 2H), 8.09 (s, 2H), 7.92 (s, 2H), 7.47 (d, J=8.0 Hz, 2H), 7.33-7.29 (m, 2H), 7.19 (d, J=6.8 Hz, 2H), 6.66 (s, 2H), 5.25-5.24 (m, 4H), 2.07 (s, 6H).

### Step 7

### (2S,2′S)-1,1′-(((((2,2′-Dimethyl-[1,1′-biphenyl]-3,3′-diyl)bis(methylene))bis(oxy))bis(5-chloro-2-((5-cyanopyridin-3-yl)methoxy)-4, 1-phenylene))bis(methylene))bis(piperidine-2-carboxylic acid) di-trifluoroacetic acid salt

To a solution of 5,5′-((((((2,2′-dimethyl-[1,1′-biphenyl]-3,3′-diyl)bis(methylene))bis(oxy))bis(4-chloro-6-formyl-3,1-phenylene))bis(oxy))bis(methylene))dinicotinonitrile (600 mg, 0.767 mmol) in MeOH (10 mL) were added (S)-piperidine-2-carboxylic acid (500 mg, 3.836 mmol) and acetic acid (0.5 mL) and the reaction mixture was stirred at 80° C. for 4-5 h. The reaction mixture was cooled to room temperature, prior to the addition of NaCNBH₃ (240 mg, 3.83 mmol) and then the reaction mixture was stirred at 80° C. for 3 h. The reaction mixture was poured into ice cold water, and solid thus precipitated was filtered and dried under vacuum. The crude neutral product was purified by prep-HPLC under acidic conditions (Mobile phase: 0.1% TFA in H₂O: ACN; Column: SUNFIRE-C18 (150*30), 5u; Gradient: (T % B): 0/30, 11/44, 11.1/98, 13/98, 13.1/30, 13/30; Flow Rate: 25 mL/min; Diluent: ACN+H₂O+THF+1% TFA) to get (2S,2′S)-1,1′-(((((2,2′-dimethyl-[1,1′-biphenyl]-3,3′-diyl)bis(methylene))bis(oxy))bis(5-chloro-2-((5-cyanopyridin-3-yl)methoxy)-4,1-phenylene))bis(methylene))bis(piperidine-2-carboxylic acid) di-trifluoroacetic acid salt (200 mg, yield: 26%, HPLC-98.34%) as a white solid. ES−, m/z 1007.2 [M−H]; [C₈₆H₅₄Cl₂N₆O₈]; ¹H NMR (500 MHz, DMSO-d₆) δ 9.69 (bs, 2H), 9.05 (dd, J=17.0 Hz, 14.0 Hz, 4H), 8.47 (s, 2H), 7.52-7.50 (m, 4H), 7.31 (t, J=5.5 Hz, 2H), 7.22 (s, 2H), 7.14 (d, J=7.5 Hz, 2H), 5.42-5.31 (m, 8H), 4.32 (d, J=12.5 Hz, 2H), 4.24 (m, 2H), 4.0 (m, 2H), 3.50 (m, 4H), 2.89 (m, 2H), 2.14 (m, 2H), 2.04 (s, 6H), 1.69-1.68 (m, 6H), 1.48 (m, 2H). m.p.=144° C. (decomp.).

### HPLC Conditions:

Column: XBRIDE C18 (150 mm×4.6 mm, 3.5 μm)
Mobile phase: 10 mM Ammonium Acetate in Water, B: 100% Acetonitrile
Flow rate: 1.0 mL/min
T/% B (min): 0/5, 1.2/5, 3.0/55, 5.0/70, 7/95, 10/95, 12/100, 14.0/5, 16/5.

### Diluent: (ACN:H₂O)

### Temp.: 25° C.

Ret. time: 5.56 min

### Purity: 98.34%.

### Prep-HPLC Conditions:

### Column: SYMMETRY-C18 (300*19), 7u

Mobile phase: Mobile phase: 0.1% TFA in H₂O:ACN
Flow rate: 20 mL/min
T/% B (min): 0/35, 8/55, 9/55, 9.1/98, 10/98, 10.1/35, 13/35

### Diluent: ACN+H₂O+THF+MeOH

### Temp.: 25° C.

### Example 36

### 2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))dimalonic acid (Cpd 36)

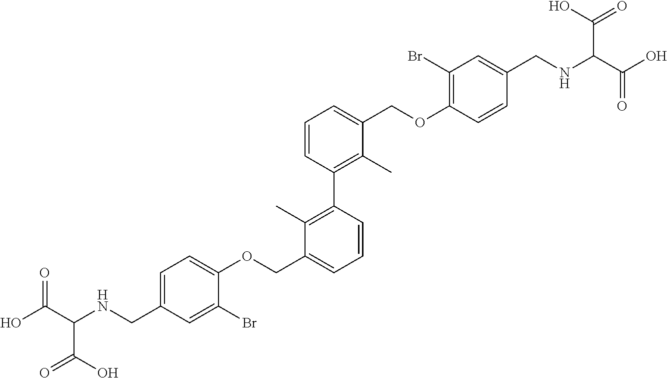 [SMILES: Cc1c(COc2ccc(CNC(C(=O)O)C(=O)O)cc2Br)cccc1-c1cccc(COc2ccc(CNC(C(=O)O)C(=O)O)cc2Br)c1C]

Compound 36 can be prepared according to the procedure described in Example 30 with 4,4′-(2,2′-dimethylbiphenyl-3,3′-diyl)bis(methylene)bis(oxy)bis(3-bromobenzaldehyde) 5 and 2-amino malonic acid, or from ((((2,2′-Dimethyl-[1,1′-biphenyl]-3,3′-diyl)bis(methylene))bis(oxy))bis(3-bromo-4,1-phenylene))dimethanamine (Cpd 37) and a diester of 2-bromomalonic acid followed by hydrolysis of the esters.

### Example 37

### ((((2,2′-Dimethyl-[1,1′-biphenyl]-3,3′-diyl)bis(methylene))bis(oxy))bis(3-bromo-4,1-phenylene))dimethanamine (Cpd 37)

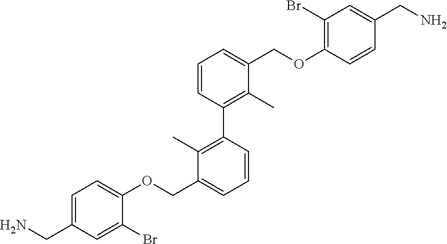 [SMILES: Cc1c(COc2ccc(CN)cc2Br)cccc1-c1cccc(COc2ccc(CN)cc2Br)c1C]

Compound 37 or an appropriate di-acid salt can be prepared in two steps from 4,4′-(2,2′-dimethylbiphenyl-3,3′-diyl)bis(methylene)bis(oxy)bis(3-bromobenzaldehyde) 5 and O-methylhydroxylamine following by reduction of the intermediate bis-dioximine with diborane.

### Example 38

### 2,2′-(((((2,2′-Dimethyl-[1,1′-biphenyl]-3,3′-diyl)bis(methylene))bis(oxy))bis(3-bromo-4,1-phenylene))bis(methylene))bis(azanediyl))bis(2-oxoacetic acid) (Cpd 38)

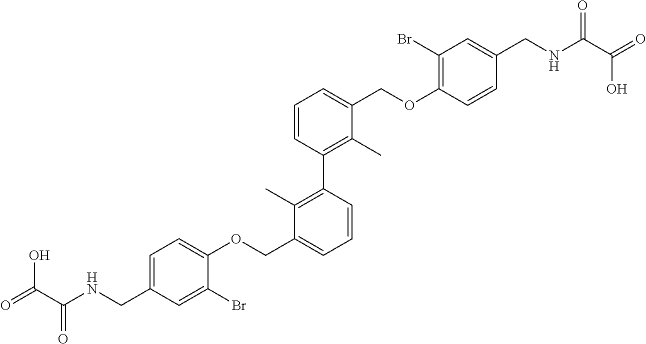 [SMILES: Cc1c(COc2ccc(CNC(=O)C(=O)O)cc2Br)cccc1-c1cccc(COc2ccc(CNC(=O)C(=O)O)cc2Br)c1C]

Compound 38 can be prepared from ((((2,2′-Dimethyl-[1,1′-biphenyl]-3,3′-diyl)bis(methylene))bis(oxy))bis(3-bromo-4,1-phenylene))dimethanamine (Cpd 37) and methyl 2-chloro-2-oxoacetate followed by hydrolysis of the bis-dimethyl ester with aqueous lithium hydroxide.

### Example 39

### 3,3′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-oxopropanoic acid) (Cpd 39)

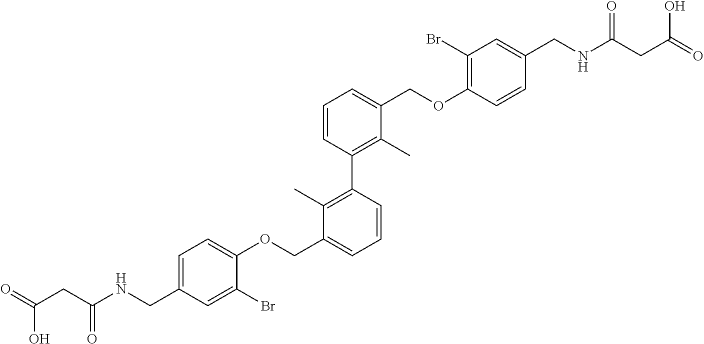 [SMILES: Cc1c(COc2ccc(CNC(=O)CC(=O)O)cc2Br)cccc1-c1cccc(COc2ccc(CNC(=O)CC(=O)O)cc2Br)c1C]

Compound 39 can be prepared from ((((2,2′-Dimethyl-[1,1′-biphenyl]-3,3′-diyl)bis(methylene))bis(oxy))bis(3-bromo-4,1-phenylene))dimethanamine (Cpd 37) and methyl 3-chloro-3-oxopropanoate followed by hydrolysis of the bis-dimethyl ester with aqueous lithium hydroxide.

### Example 40

### N,N′-(((((2,2′-Dimethyl-[1,1′-biphenyl]-3,3′-diyl)bis(methylene))bis(oxy))bis(3-bromo-4,1-phenylene))bis(methylene))dimethanesulfonamide (Cpd 40)

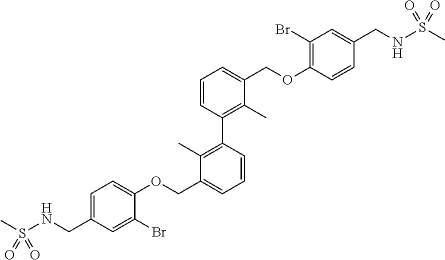

Compound 40 can be prepared from ((((2,2′-Dimethyl-[1,1′-biphenyl]-3,3′-diyl)bis(methylene))bis(oxy))bis(3-bromo-4,1-phenylene))dimethanamine (Cpd 37) and methanesulfonyl chloride.

### Example 41

### 2,2′-(((3-Bromo-4-((3′-((2-bromo-4-((hydrosulfonylamino)methyl)phenoxy)methyl)-2,2′-dimethyl-[1,1′-biphenyl]-3-yl)methoxy)benzyl)amino)sulfonyl)diacetic acid (Cpd 41)

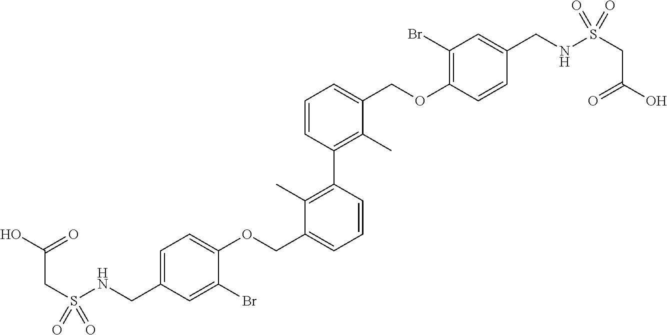 [SMILES: Cc1c(COc2ccc(CNS(=O)(=O)CC(=O)O)cc2Br)cccc1-c1cccc(COc2ccc(CNS(=O)(=O)CC(=O)O)cc2Br)c1C]

Compound 41 can be prepared from ((((2,2′-Dimethyl-[1,1′-biphenyl]-3,3′-diyl)bis(methylene))bis(oxy))bis(3-bromo-4,1-phenylene))dimethanamine (Cpd 37) and methyl 2-(chlorosulfonyl)acetate followed by hydrolysis of the bis-dimethyl ester with aqueous lithium hydroxide.

### Example 42

### (2S,2′S)-1,1′-(((((2,2′-Bis(trifluoromethyl)-[1,1′-biphenyl]-3,3′-diyl)bis(methylene))bis(oxy))bis(3-bromo-4,1-phenylene))bis(methylene))bis(piperidine-2-carboxylic acid) (Cpd 42)

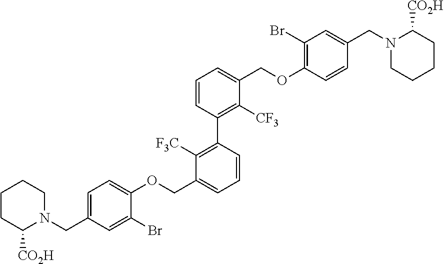 [SMILES: O=C(O)[C@@H]1CCCCN1Cc1ccc(OCc2cccc(-c3cccc(COc4ccc(CN5CCCC[C@H]5C(=O)O)cc4Br)c3C(F)(F)F)c2C(F)(F)F)c(Br)c1]

Compound 42 can be prepared according to the procedure described in Example 1 with 4,4′-(((2,2′-bis(trifluoromethyl)-[1,1′-biphenyl]-3,3′-diyl)bis(methylene))bis(oxy))bis(3-bromobenzaldehyde) and (S)-piperidine-2-carboxylic acid.

### Example 43

### (2S,2′S)-1,1′-(((((2,2′-Dicyano-[1,1′-biphenyl]-3,3′-diyl)bis(methylene))bis(oxy))bis(3-bromo-4,1-phenylene))bis(methylene))bis(piperidine-2-carboxylic acid) (Cpd 43)

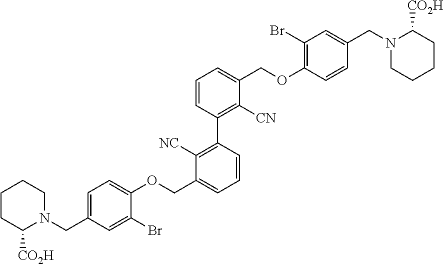 [SMILES: N#Cc1c(COc2ccc(CN3CCCC[C@H]3C(=O)O)cc2Br)cccc1-c1cccc(COc2ccc(CN3CCCC[C@H]3C(=O)O)cc2Br)c1C#N]

Compound 43 can be prepared according to the procedure described in Example 1 with 3,3′-bis((2-bromo-4-formylphenoxy)methyl)-[1,1′-biphenyl]-2,2′-dicarbonitrile and (S)-piperidine-2-carboxylic acid.

### Example 44

### (2S,2′S)-1,1′-(((((2,2′-Dimethyl-[1,1′-biphenyl]-3,3′-diyl)bis(methylene))bis(oxy))bis(2-((5-cyanopyridin-3-yl)methoxy)-5-fluoro-4,1-phenylene))bis(methylene))bis(piperidine-2-carboxylic acid) di-trifluoroacetic acid salt (Cpd 44)

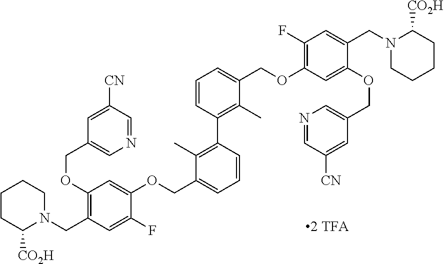 [SMILES: Cc1c(COc2cc(OCc3cncc(C#N)c3)c(CN3CCCC[C@H]3C(=O)O)cc2F)cccc1-c1cccc(COc2cc(OCc3cncc(C#N)c3)c(CN3CCCC[C@H]3C(=O)O)cc2F)c1C]

Compound 44 can be prepared according to the procedure described in Example 35 replacing 5-chloro-2,4-dihydroxybenzaldehyde with 5-fluoro-2,4-dihydroxybenzaldehyde.

### Example 45

### 2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-methylmalonic acid) (Cpd 45)

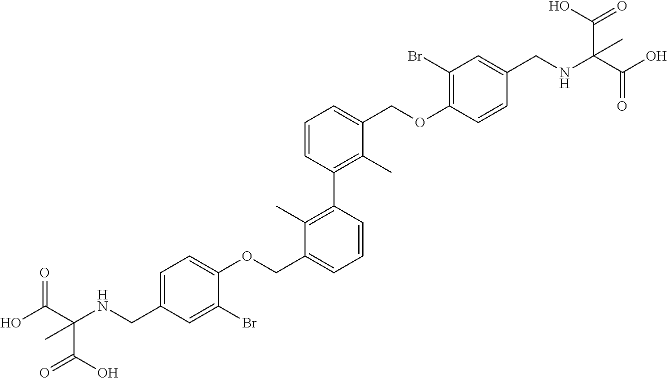 [SMILES: Cc1c(COc2ccc(CNC(C)(C(=O)O)C(=O)O)cc2Br)cccc1-c1cccc(COc2ccc(CNC(C)(C(=O)O)C(=O)O)cc2Br)c1C]

Compound 45 can be prepared according to the procedure described in Example 30 with 2-amino-2-methylmalonic acid, or from ((((2,2′-Dimethyl-[1,1′-biphenyl]-3,3′-diyl)bis(methylene))bis(oxy))bis(3-bromo-4,1-phenylene))dimethanamine (Cpd 37) and a diester of 2-bromo-2-methylmalonic acid followed by hydrolysis of the esters.

### Example 46

### 2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-hydroxy-2-(hydroxymethyl)propanoic acid) (Cpd 46)

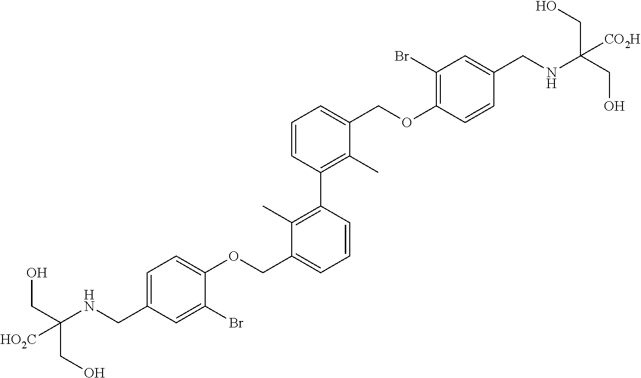 [SMILES: Cc1c(COc2ccc(CNC(CO)(CO)C(=O)O)cc2Br)cccc1-c1cccc(COc2ccc(CNC(CO)(CO)C(=O)O)cc2Br)c1C]

Compound 46 or an appropriate di-acid salt can be prepared according to the procedure described in Example 17 from 4,4′-(((2,2′-dimethyl-[1,1′-biphenyl]-3,3′-diyl)bis(methylene))bis(oxy))bis(3-bromobenzaldehyde) 5 and 2-amino-3-hydroxy-2-(hydroxymethyl)propanoic acid.

### Example 47

### 1,1′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(cyclopropane-1-carboxylic acid) di-trifluoroacetic acid (Cpd 47)

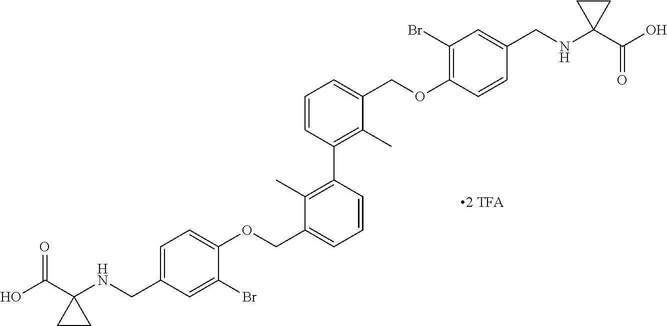 [SMILES: Cc1c(COc2ccc(CNC3(C(=O)O)CC3)cc2Br)cccc1-c1cccc(COc2ccc(CNC3(C(=O)O)CC3)cc2Br)c1C]

Compound 47 can be prepared according to the procedure described in Example 14 with 4,4′-(2,2′-dimethylbiphenyl-3,3′-diyl)bis(methylene)bis(oxy)bis(3-bromobenzaldehyde) 5 and 1-aminocyclopropane-1-carboxylic acid.

### Example 48

### 1,1′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(cyclobutane-1-carboxylic acid) di-trifluoroacetic acid (Cpd 48)

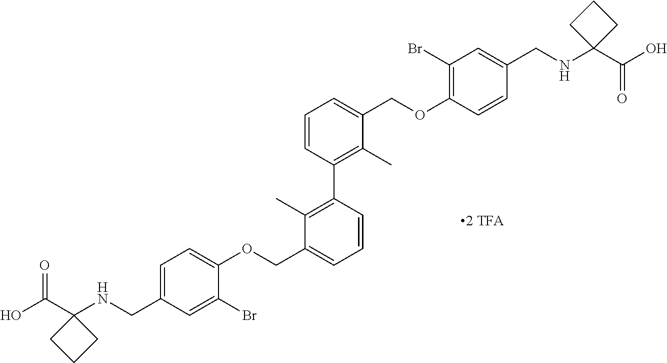 [SMILES: Cc1c(COc2ccc(CNC3(C(=O)O)CCC3)cc2Br)cccc1-c1cccc(COc2ccc(CNC3(C(=O)O)CCC3)cc2Br)c1C]

Compound 48 can be prepared according to the procedure described in Example 14 with 4,4′-(2,2′-dimethylbiphenyl-3,3′-diyl)bis(methylene)bis(oxy)bis(3-bromobenzaldehyde) 5 and 1-aminocyclobutane-1-carboxylic acid.

### Example 49

### 1,1′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(cyclopentane-1-carboxylic acid) di-trifluoroacetic acid (Cpd 49)

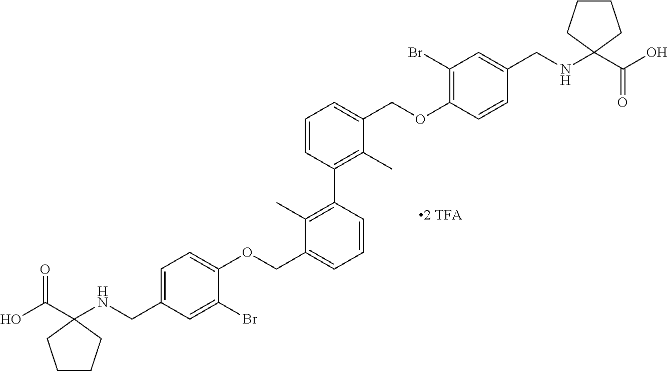 [SMILES: Cc1c(COc2ccc(CNC3(C(=O)O)CCCC3)cc2Br)cccc1-c1cccc(COc2ccc(CNC3(C(=O)O)CCCC3)cc2Br)c1C]

Compound 49 can be prepared according to the procedure described in Example 14 with 4,4′-(2,2′-dimethylbiphenyl-3,3′-diyl)bis(methylene)bis(oxy)bis(3-bromobenzaldehyde) 5 and 1-aminocyclopentane-1-carboxylic acid.

### Example 50

### (2S,2′S)-1,1′-(((((2,2′-Dichloro-[1,1′-biphenyl]-3,3′-diyl)bis(methylene))bis(oxy))bis(3-(trifluoromethyl)-4,1-phenylene))bis(methylene))bis(piperidine-2-carboxylic acid) (Cpd 50)

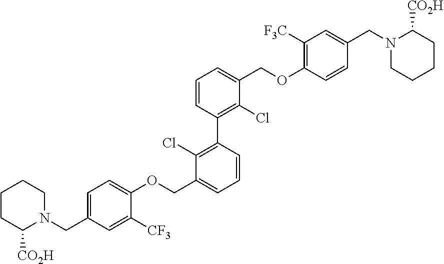 [SMILES: O=C(O)[C@@H]1CCCCN1Cc1ccc(OCc2cccc(-c3cccc(COc4ccc(CN5CCCC[C@H]5C(=O)O)cc4C(F)(F)F)c3Cl)c2Cl)c(C(F)(F)F)c1]

Compound 50 can be prepared according to the procedure described in Example 1 with 4,4′-(((2,2′-dichloro-[1,1′-biphenyl]-3,3′-diyl)bis(methylene))bis(oxy))bis(3-(trifluoromethyl)benzaldehyde) and (S)-piperidine-2-carboxylic acid.

### Example 51

### 2,2′-((((((2,2′-Dichloro-[1,1′-biphenyl]-3,3′-diyl)bis(methylene))bis(oxy))bis(3-(trifluoromethyl)-4,1-phenylene))bis(methylene))bis(azanediyl))bis(2-methylpropanoic acid) di-trifluoroacetic acid salt (Cpd 51)

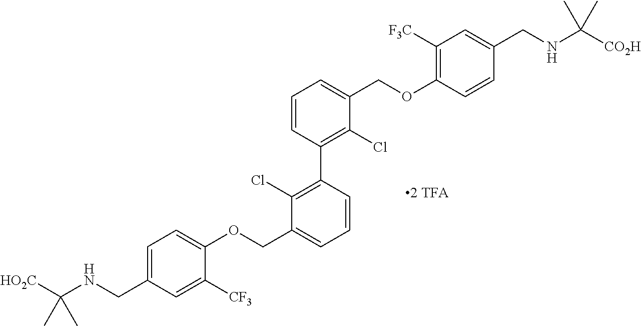 [SMILES: CC(C)(NCc1ccc(OCc2cccc(-c3cccc(COc4ccc(CNC(C)(C)C(=O)O)cc4C(F)(F)F)c3Cl)c2Cl)c(C(F)(F)F)c1)C(=O)O]

Compound 51 can be prepared according to the procedure described in Example 17 with 4,4′-(((2,2′-dichloro-[1,1′-biphenyl]-3,3′-diyl)bis(methylene))bis(oxy))bis(3-(trifluoromethyl)benzaldehyde) and 2-amino-2-methylpropanoic acid.

### Example 52

### (2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(N-(2-(dimethylamino)ethyl)-N-methylpiperidine-2-carboxamide) (Cpd 52)

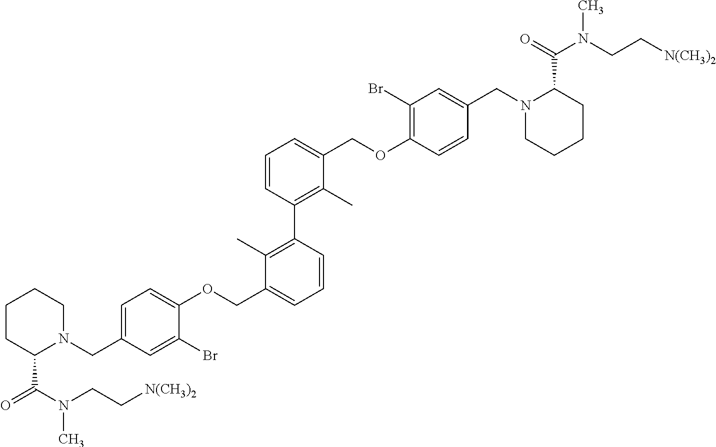 [SMILES: Cc1c(COc2ccc(CN3CCCC[C@H]3C(=O)N(C)CCN(C)C)cc2Br)cccc1-c1cccc(COc2ccc(CN3CCCC[C@H]3C(=O)N(C)CCN(C)C)cc2Br)c1C]

Compound 52 can be prepared according to the procedure described in Example 25 with from Cpd 1 and N¹,N¹,N²-trimethylethane-1,2-diamine according to the methods described for Examples 23-25.

### Example 53

### (2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(N-hydroxypiperidine-2-carboxamide) (Cpd 53)

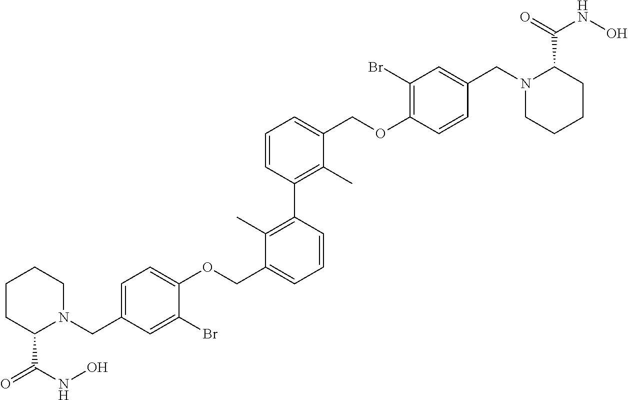 [SMILES: Cc1c(COc2ccc(CN3CCCC[C@H]3C(=O)NO)cc2Br)cccc1-c1cccc(COc2ccc(CN3CCCC[C@H]3C(=O)NO)cc2Br)c1C]

Compound 53 can be prepared according to the procedure described in Example 23 with from Cpd 1 and hydroxylamine according to the methods described for Examples 23-25.

### Example 54

### (2S,2′S)-1,1′-(((((2,2′-dimethyl-[1,1′-biphenyl]-3,3′-diyl)bis(methylene))bis(oxy))bis(3-bromo-4,1-phenylene))bis(methylene))bis(N-methoxypiperidine-2-carboxamide) (Cpd 54)

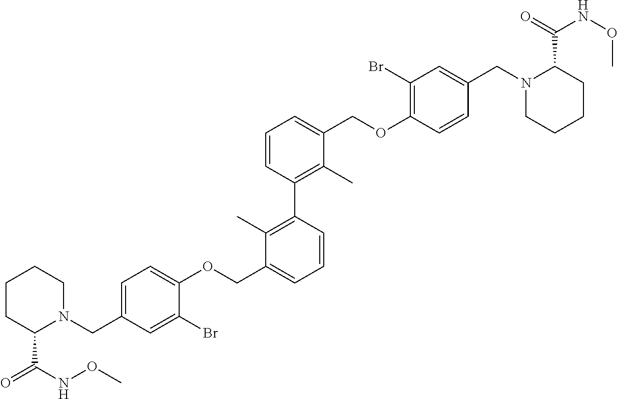 [SMILES: CONC(=O)[C@@H]1CCCCN1Cc1ccc(OCc2cccc(-c3cccc(COc4ccc(CN5CCCC[C@H]5C(=O)NOC)cc4Br)c3C)c2C)c(Br)c1]

Compound 54 can be prepared according to the procedure described in Example 23 with from Cpd 1 and O-methylhydroxylamine according to the methods described for Examples 23-25.

### Example 55

### 1,1′-((2S,2′S)-(((((2,2′-Dimethyl-[1,1′-biphenyl]-3,3′-diyl)bis(methylene))bis(oxy))bis(3-bromo-4,1-phenylene))bis(methylene))bis(piperidine-1,2-diyl))bis(N-(methylsulfonyl)formamide) (Cpd 55)

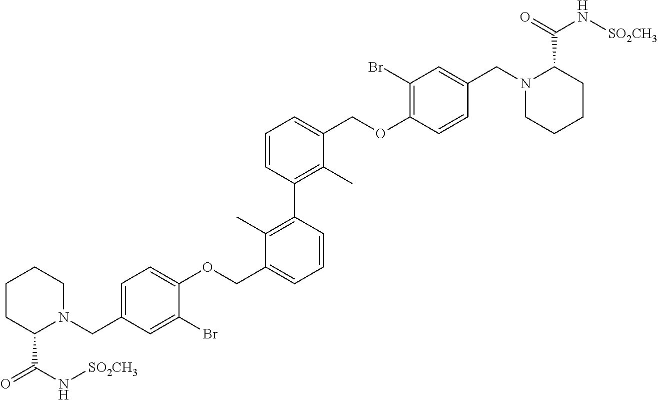 [SMILES: Cc1c(COc2ccc(CN3CCCC[C@H]3C(=O)NS(C)(=O)=O)cc2Br)cccc1-c1cccc(COc2ccc(CN3CCCC[C@H]3C(=O)NS(C)(=O)=O)cc2Br)c1C]

Compound 55 can be prepared according to the procedure described in Example 23 with from Cpd 1 and methanesulfonamide according to the methods described for Examples 23-25.

### Example 56

### 2,2′-((((((2,2′-dimethyl-[1,1′-biphenyl]-3,3′-diyl)bis(methylene))bis(oxy))bis(3-(difluoromethyl)-4,1-phenylene))bis(methylene))bis(azanediyl))bis(2-methylpropane-1,3-diol) di-trifluoroacetic acid salt (Cpd 56)

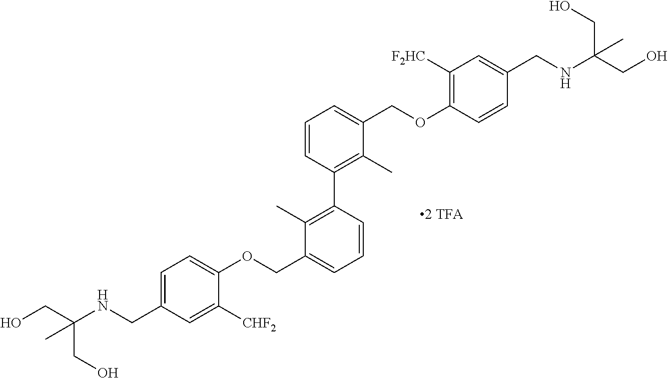 [SMILES: Cc1c(COc2ccc(CNC(C)(CO)CO)cc2C(F)F)cccc1-c1cccc(COc2ccc(CNC(C)(CO)CO)cc2C(F)F)c1C]

Compound 56 can be prepared according to the procedure described in Example 33 from 4,4′-(2,2′-dimethylbiphenyl-3,3′-diyl)bis(methylene)bis(oxy)bis(3-(difluoromethyl) benzaldehyde) 46 and 2-amino-2-methylpropane-1,3-diol.

### Example 57

### 2,2′-((((((2,2′-dimethyl-[1,1′-biphenyl]-3,3′-diyl)bis(methylene))bis(oxy))bis(3-(difluoromethyl)-4,1-phenylene))bis(methylene))bis(methylazanediyl))bis(2-methylpropane-1,3-diol) di-trifluoroacetic acid salt (Cpd 57)

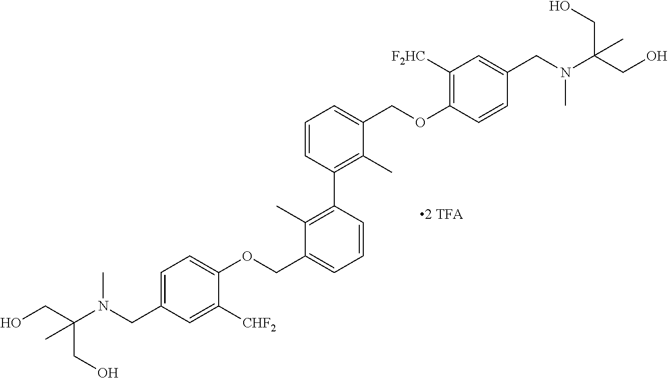 [SMILES: Cc1c(COc2ccc(CN(C)C(C)(CO)CO)cc2C(F)F)cccc1-c1cccc(COc2ccc(CN(C)C(C)(CO)CO)cc2C(F)F)c1C]

Compound 57 can be prepared according to the procedure described in Example 33 from 4,4′-(2,2′-dimethylbiphenyl-3,3′-diyl)bis(methylene)bis(oxy)bis(3-(difluoromethyl) benzaldehyde) 46 and 2-methyl-2-(methylamino)propane-1,3-diol.

### Example 58

### 2,2′-((((((2,2′-dichloro-[1,1′-biphenyl]-3,3′-diyl)bis(methylene))bis(oxy))bis(3-bromo-4,1-phenylene))bis(methylene))bis(azanediyl))bis(2-methylpropane-1,3-diol) di-trifluoroacetic acid salt (Cpd 58)

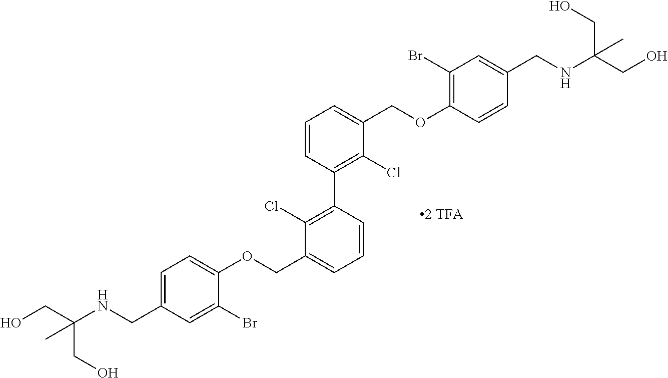 [SMILES: CC(CO)(CO)NCc1ccc(OCc2cccc(-c3cccc(COc4ccc(CNC(C)(CO)CO)cc4Br)c3Cl)c2Cl)c(Br)c1]

Compound 58 can be prepared according to the procedure described in Example 33 from 4,4′-(((2,2′-dichloro-[1,1′-biphenyl]-3,3′-diyl)bis(methylene))bis(oxy))bis(3-bromobenzaldehyde) 16 and 2-amino-2-methylpropane-1,3-diol.

### Example 59

### 2,2′-((((((2,2′-dichloro-[1,1′-biphenyl]-3,3′-diyl)bis(methylene))bis(oxy))bis(3-(difluoromethyl)-4,1-phenylene))bis(methylene))bis(azanediyl))bis(2-methylpropane-1,3-diol) di-trifluoroacetic acid salt (Cpd 59)

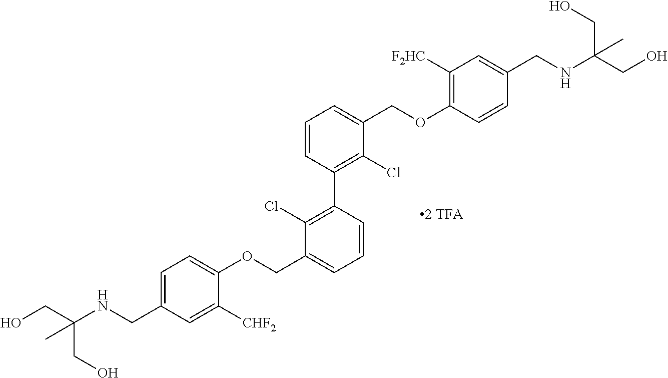 [SMILES: CC(CO)(CO)NCc1ccc(OCc2cccc(-c3cccc(COc4ccc(CNC(C)(CO)CO)cc4C(F)F)c3Cl)c2Cl)c(C(F)F)c1]

Compound 59 can be prepared according to the procedure described in Example 33 from 4,4′-(((2,2′-dichloro-[1,1′-biphenyl]-3,3′-diyl)bis(methylene))bis(oxy))bis(3-(difluoromethyl)benzaldehyde) and 2-amino-2-methylpropane-1,3-diol.

### Example 60

### 2,2′-((((((2,2′-dimethyl-[1,1′-biphenyl]-3,3′-diyl)bis(methylene))bis(oxy))bis(3-(difluoromethyl)-4,1-phenylene))bis(methylene))bis(azanediyl))bis(2-(hydroxymethyl)propane-1,3-diol) di-trifluoroacetic acid salt (Cpd 60)

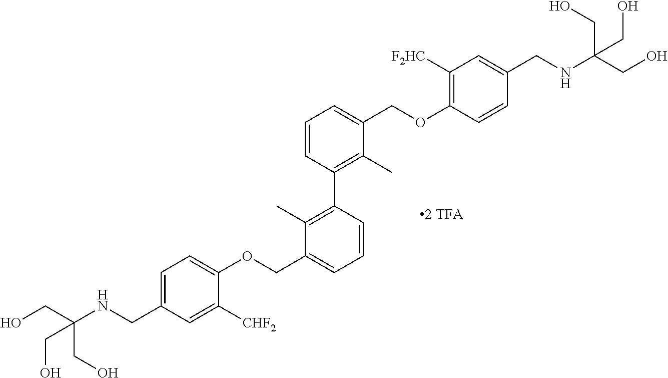 [SMILES: Cc1c(COc2ccc(CNC(CO)(CO)CO)cc2C(F)F)cccc1-c1cccc(COc2ccc(CNC(CO)(CO)CO)cc2C(F)F)c1C]

Compound 60 can be prepared according to the procedure described in Example 33 from 4,4′-(2,2′-dimethylbiphenyl-3,3′-diyl)bis(methylene)bis(oxy)bis(3-(difluoromethyl) benzaldehyde) 46 and 2-amino-2-(hydroxymethyl)propane-1,3-diol.

### Example 61

### (2S,2′S)-1,1′-(((((2,2′-dichloro-[1,1′-biphenyl]-3,3′-diyl)bis(methylene))bis(oxy))bis(3-(difluoromethyl)-4,1-phenylene))bis(methylene))bis(piperidine-2-carboxylic acid) (Cpd 61)

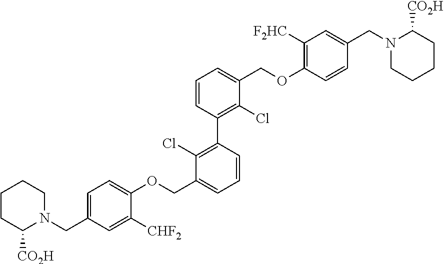 [SMILES: O=C(O)[C@@H]1CCCCN1Cc1ccc(OCc2cccc(-c3cccc(COc4ccc(CN5CCCC[C@H]5C(=O)O)cc4C(F)F)c3Cl)c2Cl)c(C(F)F)c1]

Compound 61 can be prepared according to the procedure described in Example 1 from 4,4′-(((2,2′-dichloro-[1,1′-biphenyl]-3,3′-diyl)bis(methylene))bis(oxy))bis(3-(difluoromethyl)benzaldehyde) and (S)-piperidine-2-carboxylic acid.

### Example 62

### (2S,2′S)-1,1′-(((((2,2′-dichloro-[1,1′-biphenyl]-3,3′-diyl)bis(methylene))bis(oxy))bis(2-((5-cyanopyridin-3-yl)methoxy)-5-(difluoromethyl)-4,1-phenylene))bis(methylene))bis(piperidine-2-carboxylic acid) di-trifluoroacetic acid salt (Cpd 62)

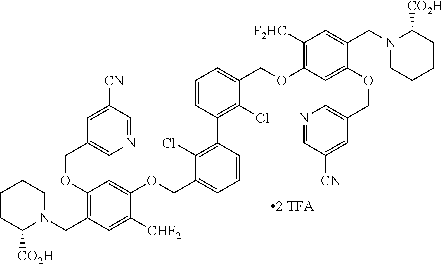 [SMILES: N#Cc1cncc(COc2cc(OCc3cccc(-c4cccc(COc5cc(OCc6cncc(C#N)c6)c(CN6CCCC[C@H]6C(=O)O)cc5C(F)F)c4Cl)c3Cl)c(C(F)F)cc2CN2CCCC[C@H]2C(=O)O)c1]

Compound 62 can be prepared according to the procedure described in Example 35 replacing 3,3′-bis(bromomethyl)-2,2′-dimethyl-1,1′-biphenyl with 3,3′-bis(bromomethyl)-2,2′-dichloro-1,1′-biphenyl and 5-chloro-2,4-dihydroxybenzaldehyde with 5-(difluoromethyl)-2,4-dihydroxybenzaldehyde.

### PD-L1/PD-1 Interaction Assay

The ability of compounds to block PD-L1/PD-1 complex formation was evaluated using biochemical interaction assay based on the ELISA platform.

EIA/RIA high binding 96-well plates (Costar) were coated with human PD-1-Fc protein diluted in PBS to 0.5 μg/ml overnight at 4° C. The PD-1-Fc coated plates were washed in PBST and blocked in blocking buffer (2% BSA in PBST). Serially diluted compounds were pre-incubated with PD-L1-Fc-biotin (0.1 μg/ml final) for 2 hours on a shaker at room temperature. The PD-1-Fc coated plates were washed with PBST and solutions containing PD-L1 and compounds were added to the plates. The PD-1-Fc coated plates were incubated with PD-L1 and compound solution for 2 hours on a shaker at room temperature. After the incubation unbound PD-L1 was washed away with PBST and plates were incubated with the blocking buffer for 10 minutes. The bound PD-L1-Fc-biotin was detected with streptavidin-HRP and TMB substrate. The plates were read at OD450 nm in a plate reader (i3 from Molecular Devices) and percentages of inhibition of PD-L1/PD-1 complex formation were calculated.

PD-1-Fc protein was made at Polaris and PD-L1-Fc was purchased from BPS Bioscience. The IC₅₀ values measured in the assay and shown in Table 1 were in the following ranges: A=0.0001-0.010 μM; B=0.011-0.10 μM; C=0.11-1.00 μM; D=1.01-30.0 μM; E>30.0 μM or no activity observed at concentrations >30.0 μM.

TABLE 1

| Example # | PD-1/PD-L1 Interaction Assay IC₅₀ |
|---|---|
| 2 | B |
| 1 | A |
| 3 | B |
| 4 | E |
| 5 | A |
| 6 | B |
| 7 | A |
| 8 | D |
| 9 | A |
| 10 | C |
| 11 | B |
| 12 | B |
| 13 | A |
| 14 | A |
| 15 | A |
| 16 | B |
| 18 | A |
| 17 | A |
| 19 | C |
| 22 | B |
| 20 | D |
| 21 | D |
| 26 | B |
| 27 | C |
| 29 | A |
| 28 | C |
| 30 | C |
| 31 | B |
| 32 | A |
| 33 | A |
| 25 | D |
| 24 | C |
| 23 | D |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled.

1. A compound according to Formula (I): 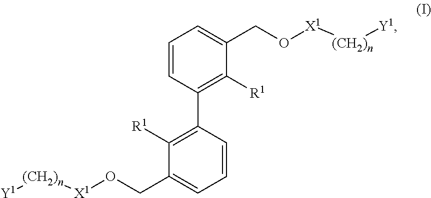
wherein
X¹ is aryl or heteroaryl,
Y¹ is NH(R²), N(R²)(R³) or heterocyclyl,
R¹ is H, CN, halogen, (C₁-C₈)alkyl or (C₁-C₈)haloalkyl,
R² and R³ are independently H, (C₁-C₈)alkyl, C(O)alkyl, C(O)CO₂H, or (SO₂)alkyl, or R² and R³ combine together with the nitrogen atom to form a heterocyclyl,
R⁴ is H, OH or (C₁-C₈)alkyl, and
n is an integer 0, 1 or 2,
wherein any alkyl, aryl, heterocyclyl or heteroaryl is optionally substituted with 1, 2 or 3 groups selected from OH, CN, NO₂, halogen, (C₁-C₃)alkyl, (C₁-C₃)haloalkyl, (C₃-C₆)cycloalkyl, alkenyl, alkynyl, O—(C₁-C₃)alkyl, O—(C₁-C₃)alkyl, O—(C₁-C₃)haloalkyl, O-(alkylene)aryl, O-(alkylene)heteroaryl, S—(C₁-C₈)alkyl, S—(C₁-C₈)haloalkyl, (SO₂)alkyl, C(O)OR⁴, C(O)NR⁴R⁴, C(O)NR⁴OR⁴, C(O)NR⁴(alkylene)OH, C(O)NR⁴(alkylene)N(R⁴)₂, C(O)NR⁴(SO₂)R⁴, NR⁴R⁴, NR⁴(CO)OR⁴ or NH(CO)R⁴, wherein the two R⁴'s of C(O)NR⁴R⁴, NR⁴R⁴ or C(O)NR⁴OR⁴ can optionally combine together with the nitrogen atom to form a heterocyclyl,
or a stereoisomer, a tautomer or a pharmaceutically acceptable salt thereof.

2. The compound of claim 1, or a stereoisomer, a tautomer or a pharmaceutically acceptable salt thereof, wherein X¹ is aryl.

3. The compound of claim 1, or a stereoisomer, a tautomer or a pharmaceutically acceptable salt thereof, wherein X¹ is aryl optionally substituted by halogen, (C₁-C₃)alkyl or (C₁-C₃)haloalkyl.

4. The compound of claim 1, or a stereoisomer, a tautomer or a pharmaceutically acceptable salt thereof, wherein Y¹ is NH(R²) or N(R²)(R³).

5. The compound of claim 1, or a stereoisomer, a tautomer or a pharmaceutically acceptable salt thereof, wherein R¹ is (C₁-C₈)alkyl or halogen.

6. The compound of claim 5, or a stereoisomer, a tautomer or a pharmaceutically acceptable salt thereof, wherein R¹ is CH₃.

7. The compound of claim 1, or a stereoisomer, a tautomer or a pharmaceutically acceptable salt thereof, wherein R² and R³ are independently H, (C₁-C₈)alkyl or R² and R³ combine together with the nitrogen atom to form a heterocyclyl.

8. The compound of claim 7, or a stereoisomer, a tautomer or a pharmaceutically acceptable salt thereof, wherein R² and R³ are independently H or (C₁-C₈)alkyl, wherein (C₁-C₈)alkyl is optionally substituted by OH or C(O)OH.

9. The compound of claim 1, or a stereoisomer, a tautomer or a pharmaceutically acceptable salt thereof, wherein R² and R³ combine together with the nitrogen atom to form a heterocyclyl, optionally substituted by C(O)OH.

10. The compound of claim 1, or a stereoisomer, a tautomer or a pharmaceutically acceptable salt thereof, wherein “n” is 1.

11. A compound of claim 1, or a stereoisomer, a tautomer or a pharmaceutically acceptable salt thereof, selected from
(2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(piperidine-2-carboxylic acid),
(2R,2′R)-1,1′-(((((2,2′-Dimethyl-[1,1′-biphenyl]-3,3′-diyl)bis(methylene))bis(oxy))bis(3-bromo-4,1-phenylene))bis(methylene))bis(piperidine-2-carboxylic acid),
N,N′-(((((((2,2′-Dimethyl-[1,1′-biphenyl]-3,3′-diyl)bis(methylene))bis(oxy))bis(2-methoxypyridine-6,3-diyl))bis(methylene))bis(azanediyl))bis(ethane-2, 1-diyl))diacetamide,
Dimethyl 1,1′-(((((2,2′-dimethyl-[1,1′-biphenyl]-3,3′-diyl)bis(methylene))bis(oxy))bis(3-bromo-4,1-phenylene))bis(methylene))(2S,2′S)-bis(piperidine-2-carboxylate),
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(ethan-1-ol)
N,N′-(((((((2,2′-Dimethyl-[1,1′-biphenyl]-3,3′-diyl)bis(methylene))bis(oxy))bis(3-bromo-4,1-phenylene))bis(methylene))bis(azanediyl))bis(ethane-2,1-diyl))diacetamide,
(2S,2'S,4S,4′S)-1,1′-(((((2,2′-Dimethyl-[1,1′-biphenyl]-3,3′-diyl)bis(methylene))bis(oxy))bis(3-bromo-4,1-phenylene))bis(methylene))bis(4-hydroxypyrrolidine-2-carboxylic acid),
(2S,2′S)-1,1′-(((([1,1′-Biphenyl]-3,3′-diylbis(methylene))bis(oxy))bis(3-bromo-4,1-phenylene))bis(methylene))bis(piperidine-2-carboxylic acid),
(2S,2′S)-1,1′-(((((2,2′-Dichloro-[1,1′-biphenyl]-3,3′-diyl)bis(methylene))bis(oxy))bis(3-bromo-4,1-phenylene))bis(methylene))bis(piperidine-2-carboxylic acid),
(2S,2′S)-1,1′-(((((2,2′-Difluoro-[1,1′-biphenyl]-3,3′-diyl)bis(methylene))bis(oxy))bis(3-bromo-4,1-phenylene))bis(methylene))bis(piperidine-2-carboxylic acid),
(2S,2′S)-1,1′-(((((2,2′-Dimethyl-[1,1′-biphenyl]-3,3′-diyl)bis(methylene))bis(oxy))bis(3-methyl-4,1-phenylene))bis(methylene))bis(piperidine-2-carboxylic acid),
(2S,2′S)-1,1′-(((((2,2′-Dimethyl-[1,1′-biphenyl]-3,3′-diyl)bis(methylene))bis(oxy))bis(3-chloro-4,1-phenylene))bis(methylene))bis(piperidine-2-carboxylic acid),
(2S,2′S)-1,1′-(((((2,2′-Dimethyl-[1,1′-biphenyl]-3,3′-diyl)bis(methylene))bis(oxy))bis(3-(trifluoromethyl)-4,1-phenylene))bis(methylene))bis(piperidine-2-carboxylic acid),
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-methylpropanoic acid) di-trifluoroacetic acid salt,
(2S,2′S)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))dipropionic acid di-trifluoroacetic acid salt,
(2R,2′R)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))dipropionic acid ditrifluoroacetic acid salt,
(2R,2′R)-2,2′-((((((2,2′-Dimethyl-[1,1-biphenyl]-3,3′-diyl)bis(methylene))bis(oxy))bis(3-bromo-4,1-phenylene))bis(methylene))bis(azanediyl))bis(3-hydroxypropanoic acid) ditrifluoroacetic acid salt,
(2R,2′R,3S,3′S)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-hydroxybutanoic acid) di-trifluoroacetic acid salt,
(2S,2′S)-1,1′-(((((2,2′-Dimethyl-[1,1′-biphenyl]-3,3′-diyl)bis(methylene))bis(oxy))bis(3-fluoro-4,1-phenylene))bis(methylene))bis(piperidine-2-carboxylic acid),
(2S,2′S)-1,1′-(((((2,2′-Dimethyl-[1,1′-biphenyl]-3,3′-diyl)bis(methylene))bis(oxy))bis(3-methoxy-4,1-phenylene))bis(methylene))bis(piperidine-2-carboxylic acid),
(2S,2′S)-1,1′-(((((2,2′-Dimethyl-[1,1′-biphenyl]-3,3′-diyl)bis(methylene))bis(oxy))bis(3-hydroxy-4,1-phenylene))bis(methylene))bis(piperidine-2-carboxylic acid),
2,2′-((((((2,2′-Dimethyl-[1,1′-biphenyl]-3,3′-diyl)bis(methylene))bis(oxy))bis(2,6-dimethoxy-4,1-phenylene))bis(methylene))bis(azanediyl))bis(ethan-1-ol),
(2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(piperidine-2-carboxamide),
(2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(N-(2-hydroxyethyl)piperidine-2-carboxamide),
(2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(N-(2-(dimethylamino)ethyl)piperidine-2-carboxamide),
(2S,2′S)-1,1′-(((((2,2′-Dimethyl-[1,1′-biphenyl]-3,3′-diyl)bis(methylene))bis(oxy))bis(3-ethynyl-4,1-phenylene))bis(methylene))bis(piperidine-2-carboxylic acid) di-trifluoroacetic acid,
(2S,2′S)-1,1′-(((((2,2′-Dimethyl-[1,1′-biphenyl]-3,3′-diyl)bis(methylene))bis(oxy))bis(3-ethyl-4,1-phenylene))bis(methylene))bis(piperidine-2-carboxylic acid) di-trifluoroacetic acid,
(2S,2′S)-1,1′-(((((2,2′-Dimethyl-[1,1′-biphenyl]-3,3′-diyl)bis(methylene))bis(oxy))bis(3-(methylthio)-4,1-phenylene))bis(methylene))bis(piperidine-2-carboxylic acid) di-trifluoroacetic acid,
(2S,2′S)-1,1′-(((((2,2′-Dimethyl-[1,1′-biphenyl]-3,3′-diyl)bis(methylene))bis(oxy))bis(3-(difluoromethyl)-4,1-phenylene))bis(methylene))bis(piperidine-2-carboxylic acid) di-trifluoroacetic acid salt,
2,2′,2″,2′″-((((((2,2′-Dimethyl-[1,1′-biphenyl]-3,3′-diyl)bis(methylene))bis(oxy))bis(3-bromo-4,1-phenylene))bis(methylene))bis(azanetriyl))tetraacetic acid di-trifluoroacetic acid salt,
2,2′,2″,2′″-((((((2,2′-Dimethyl-[1,1′-biphenyl]-3,3′-diyl)bis(methylene))bis(oxy))bis(3-bromo-4,1-phenylene))bis(methylene))bis(azanetriyl))tetrakis(ethan-1-ol) di-trifluoroacetic acid salt,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(propane-1,3-diol) di-trifluoroacetic acid salt,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-methylpropane-1,3-diol) di-trifluoroacetic acid salt,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))dimalonic acid,
((((2,2′-Dimethyl-[1,1′-biphenyl]-3,3′-diyl)bis(methylene))bis(oxy))bis(3-bromo-4,1-phenylene))dimethanamine,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-oxoacetic acid),
3,3′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-oxopropanoic acid),
N,N′-(((((2,2′-Dimethyl-[1,1′-biphenyl]-3,3′-diyl)bis(methylene))bis(oxy))bis(3-bromo-4,1-phenylene))bis(methylene))dimethanesulfonamide,
2,2′-(((3-Bromo-4-((3′-((2-bromo-4-((hydrosulfonylamino)methyl)phenoxy)methyl)-2,2′-dimethyl-[1,1′-biphenyl]-3-yl)methoxy)benzyl)amino)sulfonyl)diacetic acid,
(2S,2′S)-1,1′-(((((2,2′-Bis(trifluoromethyl)-[1,1′-biphenyl]-3,3′-diyl)bis(methylene))bis(oxy))bis(3-bromo-4,1-phenylene))bis(methylene))bis(piperidine-2-carboxylic acid),
(2S,2′S)-1,1′-(((((2,2′-Dicyano-[1,1′-biphenyl]-3,3′-diyl)bis(methylene))bis(oxy))bis(3-bromo-4,1-phenylene))bis(methylene))bis(piperidine-2-carboxylic acid),
(2S,2′S)-1,1′-(((((2,2′-dimethyl-[1,1′-biphenyl]-3,3′-diyl)bis(methylene))bis(oxy))bis(3-cyano-4,1-phenylene))bis(methylene))bis(piperidine-2-carboxylic acid),
(2S,2′S)-1,1′-(((((2,2′-Dimethyl-[1,1′-biphenyl]-3,3′-diyl)bis(methylene))bis(oxy))bis(2-((5-cyanopyridin-3-yl)methoxy)-5-fluoro-4, 1-phenylene))bis(methylene))bis(piperidine-2-carboxylic acid),
(2S,2′S)-1,1′-(((((2,2′-Dimethyl-[1,1′-biphenyl]-3,3′-diyl)bis(methylene))bis(oxy))bis(5-chloro-2-((5-cyanopyridin-3-yl)methoxy)-4, 1-phenylene))bis(methylene))bis(piperidine-2-carboxylic acid),
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-methylmalonic acid),
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-hydroxy-2-(hydroxymethyl)propanoic acid),
1,1′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(cyclopropane-1-carboxylic acid) di-trifluoroacetic acid,
1,1′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(cyclobutane-1-carboxylic acid) di-trifluoroacetic acid,
1,1′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(cyclopentane-1-carboxylic acid) di-trifluoroacetic acid,
(2S,2′S)-1,1′-(((((2,2′-Dichloro-[1,1′-biphenyl]-3,3′-diyl)bis(methylene))bis(oxy))bis(3-(trifluoromethyl)-4,1-phenylene))bis(methylene))bis(piperidine-2-carboxylic acid),
2,2′-((((((2,2′-Dichloro-[1,1′-biphenyl]-3,3′-diyl)bis(methylene))bis(oxy))bis(3-(trifluoromethyl)-4,1-phenylene))bis(methylene))bis(azanediyl))bis(2-methylpropanoic acid) di-trifluoroacetic acid salt,
(2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(N-(2-(dimethylamino)ethyl)-N-methylpiperidine-2-carboxamide),
(2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(N-hydroxypiperidine-2-carboxamide),
1,1′-((2S,2′S)-(((((2,2′-Dimethyl-[1,1′-biphenyl]-3,3′-diyl)bis(methylene))bis(oxy))bis(3-bromo-4,1-phenylene))bis(methylene))bis(piperidine-1,2-diyl))bis(N-(methylsulfonyl)formamide),
(2S,2′S)-1,1′-(((((2,2′-dimethyl-[1,1′-biphenyl]-3,3′-diyl)bis(methylene))bis(oxy))bis(3-cyano-4,1-phenylene))bis(methylene))bis(piperidine-2-carboxylic acid) di-trifluoroacetic acid salt,
(2S,2′S)-1,1′-(((((2,2′-Dimethyl-[1,1′-biphenyl]-3,3′-diyl)bis(methylene))bis(oxy))bis(5-chloro-2-((5-cyanopyridin-3-yl)methoxy)-4, 1-phenylene))bis(methylene))bis(piperidine-2-carboxylic acid) di-trifluoroacetic acid salt,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))dimalonic acid,
((((2,2′-Dimethyl-[1,1′-biphenyl]-3,3′-diyl)bis(methylene))bis(oxy))bis(3-bromo-4,1-phenylene))dimethanamine,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-oxoacetic acid),
3,3′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-oxopropanoic acid),
N,N′-(((((2,2′-Dimethyl-[1,1′-biphenyl]-3,3′-diyl)bis(methylene))bis(oxy))bis(3-bromo-4,1-phenylene))bis(methylene))dimethanesulfonamide,
2,2′-(((3-Bromo-4-((3′-((2-bromo-4-((hydrosulfonylamino)methyl)phenoxy)methyl)-2,2′-dimethyl-[1,1′-biphenyl]-3-yl)methoxy)benzyl)amino)sulfonyl)diacetic acid (Cpd 41), (2S,2′S)-1,1′-(((((2,2′-Bis(trifluoromethyl)-[1,1′-biphenyl]-3,3′-diyl)bis(methylene))bis(oxy))bis(3-bromo-4,1-phenylene))bis(methylene))bis(piperidine-2-carboxylic acid),
(2S,2′S)-1,1′-(((((2,2′-Dicyano-[1,1′-biphenyl]-3,3′-diyl)bis(methylene))bis(oxy))bis(3-bromo-4,1-phenylene))bis(methylene))bis(piperidine-2-carboxylic acid),
(2S,2′S)-1,1′-(((((2,2′-Dimethyl-[1,1′-biphenyl]-3,3′-diyl)bis(methylene))bis(oxy))bis(2-((5-cyanopyridin-3-yl)methoxy)-5-fluoro-4, 1-phenylene))bis(methylene))bis(piperidine-2-carboxylic acid) di-trifluoroacetic acid salt,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-methylmalonic acid),
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-hydroxy-2-(hydroxymethyl)propanoic acid),
1,1′-((((((2,2′-Dimethyl-[1,1-biphenyl]-3,3′-diyl)bis(methylene))bis(oxy))bis(3-bromo-4,1-phenylene))bis(methylene))bis(azanediyl))bis(cyclopropane-1-carboxylic acid) di-trifluoroacetic acid,
1,1′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(cyclobutane-1-carboxylic acid) di-trifluoroacetic acid,
1,1′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(cyclopentane-1-carboxylic acid) di-trifluoroacetic acid,
(2S,2′S)-1,1′-(((((2,2′-Dichloro-[1,1′-biphenyl]-3,3′-diyl)bis(methylene))bis(oxy))bis(3-(trifluoromethyl)-4,1-phenylene))bis(methylene))bis(piperidine-2-carboxylic acid),
2,2′-((((((2,2′-Dichloro-[1,1′-biphenyl]-3,3′-diyl)bis(methylene))bis(oxy))bis(3-(trifluoromethyl)-4,1-phenylene))bis(methylene))bis(azanediyl))bis(2-methylpropanoic acid) di-trifluoroacetic acid salt,
(2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(N-(2-(dimethylamino)ethyl)-N-methylpiperidine-2-carboxamide),
(2S,2′S)-1,1′-(((((2,2′-Dimethyl-[1,1′-biphenyl]-3,3′-diyl)bis(methylene))bis(oxy))bis(3-bromo-4,1-phenylene))bis(methylene))bis(N-hydroxypiperidine-2-carboxamide),
(2S,2′S)-1,1′-(((((2,2′-dimethyl-[1,1′-biphenyl]-3,3′-diyl)bis(methylene))bis(oxy))bis(3-bromo-4,1-phenylene))bis(methylene))bis(N-methoxypiperidine-2-carboxamide),
1,1′-((2S,2′S)-(((((2,2′-Dimethyl-[1,1′-biphenyl]-3,3′-diyl)bis(methylene))bis(oxy))bis(3-bromo-4,1-phenylene))bis(methylene))bis(piperidine-1,2-diyl))bis(N-(methylsulfonyl)formamide),
2,2′-((((((2,2′-dimethyl-[1,1′-biphenyl]-3,3′-diyl)bis(methylene))bis(oxy))bis(3-(difluoromethyl)-4,1-phenylene))bis(methylene))bis(azanediyl))bis(2-methylpropane-1,3-diol) di-trifluoroacetic acid salt,
2,2′-((((((2,2′-dimethyl-[1,1′-biphenyl]-3,3′-diyl)bis(methylene))bis(oxy))bis(3-(difluoromethyl)-4,1-phenylene))bis(methylene))bis(methylazanediyl))bis(2-methylpropane-1,3-diol) di-trifluoroacetic acid salt,
2,2′-((((((2,2′-dichloro-[1,1-biphenyl]-3,3′-diyl)bis(methylene))bis(oxy))bis(3-bromo-4,1-phenylene))bis(methylene))bis(azanediyl))bis(2-methylpropane-1,3-diol) di-trifluoroacetic acid salt,
2,2′-((((((2,2′-dichloro-[1,1′-biphenyl]-3,3′-diyl)bis(methylene))bis(oxy))bis(3-(difluoromethyl)-4,1-phenylene))bis(methylene))bis(azanediyl))bis(2-methylpropane-1,3-diol) di-trifluoroacetic acid salt,
2,2′-((((((2,2′-dimethyl-[1,1′-biphenyl]-3,3′-diyl)bis(methylene))bis(oxy))bis(3-(difluoromethyl)-4,1-phenylene))bis(methylene))bis(azanediyl))bis(2-(hydroxymethyl)propane-1,3-diol) di-trifluoroacetic acid salt,
(2S,2′S)-1,1′-(((((2,2′-dichloro-[1,1′-biphenyl]-3,3′-diyl)bis(methylene))bis(oxy))bis(3-(difluoromethyl)-4, 1-phenylene))bis(methylene))bis(piperidine-2-carboxylic acid), and
(2S,2′S)-1,1′-(((((2,2′-dichloro-[1,1′-biphenyl]-3,3′-diyl)bis(methylene))bis(oxy))bis(2-((5-cyanopyridin-3-yl)methoxy)-5-(difluoromethyl)-4, 1-phenylene))bis(methylene))bis(piperidine-2-carboxylic acid) di-trifluoroacetic acid salt.

12. The compound of claim 11 selected from
(2S,2′S)-1,1′-(((((2,2′-Dichloro-[1,1′-biphenyl]-3,3′-diyl)bis(methylene))bis(oxy))bis(3-bromo-4,1-phenylene))bis(methylene))bis(piperidine-2-carboxylic acid),
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(propane-1,3-diol) di-trifluoroacetic acid salt,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-methylpropanoic acid) di-trifluoroacetic acid salt,
(2R,2′R)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-hydroxypropanoic acid) di-trifluoroacetic acid salt,
(2S,2′S)-1,1′-(((((2,2′-dimethyl-[1,1′-biphenyl]-3,3′-diyl)bis(methylene))bis(oxy))bis(3-bromo-4,1-phenylene))bis(methylene))bis(piperidine-2-carboxylic acid),
(2S,2′S)-1,1′-(4,4′-(2,2′-dimethylbiphenyl-3,3′-diyl)bis(methylene)bis(oxy)bis(3-(methylthio)-4,1-phenylene))bis(methylene)dipiperidine-2-carboxylic acid di-trifluoroacetic acid salt,
(2S,2′S)-1,1′-(((((2,2′-Dimethyl-[1,1′-biphenyl]-3,3′-diyl)bis(methylene))bis(oxy))bis(3-(trifluoromethyl)-4,1-phenylene))bis(methylene))bis(piperidine-2-carboxylic acid),
(2S,2'S,4S,4′S)-1,1′-(((((2,2′-Dimethyl-[1,1′-biphenyl]-3,3′-diyl)bis(methylene))bis(oxy))bis(3-bromo-4,1-phenylene))bis(methylene))bis(4-hydroxypyrrolidine-2-carboxylic acid),
(2S,2′S)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))dipropionic acid di-trifluoroacetic acid salt,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-methylpropane-1,3-diol) di-trifluoroacetic acid salt,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(ethan-1-ol) di-trifluoroacetic acid salt, and
(2R,2′R,3S,3′S)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-hydroxybutanoic acid) di-trifluoroacetic acid salt.

13. The compound of claim 1, wherein the pharmaceutically acceptable salt of the Formula (I) compound is an organic or inorganic acid salt selected from the group consisting of acetate, trifluoroacetate, mesylate, sulfate, citrate, oxalate, hydrochloride, dihydrochloride, isothionate, lactate, laurate, maleate, fumarate, arginine, lysine, meglumine, sodium, and potassium.

14. A pharmaceutical composition comprising (i) a therapeutically effective amount of at least one compound or a stereoisomer, tautomer or pharmaceutically acceptable salt thereof according to claim 1; (ii) in combination with a pharmaceutically acceptable carrier, diluent or excipient.

15. A method of modulating PD-L1 or PD-1 activity, comprising contacting a cell with an effective amount of a compound of Formula I or a stereoisomer, tautomer or pharmaceutically acceptable salt thereof to increase immune cell activity or for reducing down-modulation of immune cells.

16. The method of claim 15 wherein the compound of Formula I is selected from
(2S,2′S)-1,1′-(((((2,2′-Dichloro-[1,1′-biphenyl]-3,3′-diyl)bis(methylene))bis(oxy))bis(3-bromo-4,1-phenylene))bis(methylene))bis(piperidine-2-carboxylic acid),
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(propane-1,3-diol) di-trifluoroacetic acid salt,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-methylpropanoic acid) di-trifluoroacetic acid salt,
(2R,2′R)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-hydroxypropanoic acid) di-trifluoroacetic acid salt,
(2S,2′S)-1,1′-(((((2,2′-dimethyl-[1,1′-biphenyl]-3,3′-diyl)bis(methylene))bis(oxy))bis(3-bromo-4,1-phenylene))bis(methylene))bis(piperidine-2-carboxylic acid),
(2S,2′S)-1,1′-(4,4′-(2,2′-dimethylbiphenyl-3,3′-diyl)bis(methylene)bis(oxy)bis(3-(methylthio)-4,1-phenylene))bis(methylene)dipiperidine-2-carboxylic acid di-trifluoroacetic acid salt,
(2S,2′S)-1,1′-(((((2,2′-Dimethyl-[1,1′-biphenyl]-3,3′-diyl)bis(methylene))bis(oxy))bis(3-(trifluoromethyl)-4,1-phenylene))bis(methylene))bis(piperidine-2-carboxylic acid),
(2S,2'S,4S,4′S)-1,1′-(((((2,2′-Dimethyl-[1,1′-biphenyl]-3,3′-diyl)bis(methylene))bis(oxy))bis(3-bromo-4,1-phenylene))bis(methylene))bis(4-hydroxypyrrolidine-2-carboxylic acid),
(2S,2′S)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))dipropionic acid di-trifluoroacetic acid salt,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-methylpropane-1,3-diol) di-trifluoroacetic acid salt,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(ethan-1-ol) di-trifluoroacetic acid salt, and
(2R,2′R,3S,3′S)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-hydroxybutanoic acid) di-trifluoroacetic acid salt.

17. A method for treating a PD-1/PD-L1 dependent condition in a mammal in need thereof comprising administering to the mammal a therapeutically effective amount of at least one compound according to Formula (I) or a stereoisomer, tautomer or pharmaceutically acceptable salt thereof.

18. The method of claim 17 wherein the PD-1/PD-L1 dependent condition is colon cancer, gastric cancer, esophageal cancer, head and neck tumors, thyroid cancer, lung cancer, leukemia, Hodgkin's lymphoma, non-Hodgkin's lymphoma, hairy cell lymphoma, Burkitt's lymphoma, B-cell lymphoma, acute T-cell leukemia, T-cell lymphoma, pancreatic cancer, melanoma, multiple melanoma, bladder cancer, brain cancer, CNS cancer, renal cancer, prostate cancer, ovarian cancer or breast cancer.

19. The method of claim 17 wherein the compound of Formula I is selected from
(2S,2′S)-1,1′-(((((2,2′-Dichloro-[1,1′-biphenyl]-3,3′-diyl)bis(methylene))bis(oxy))bis(3-bromo-4,1-phenylene))bis(methylene))bis(piperidine-2-carboxylic acid),
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(propane-1,3-diol) di-trifluoroacetic acid salt,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-methylpropanoic acid) di-trifluoroacetic acid salt,
(2R,2′R)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-hydroxypropanoic acid) di-trifluoroacetic acid salt,
(2S,2′S)-1,1′-(((((2,2′-dimethyl-[1,1′-biphenyl]-3,3′-diyl)bis(methylene))bis(oxy))bis(3-bromo-4,1-phenylene))bis(methylene))bis(piperidine-2-carboxylic acid),
(2S,2′S)-1,1′-(4,4′-(2,2′-dimethylbiphenyl-3,3′-diyl)bis(methylene)bis(oxy)bis(3-(methylthio)-4,1-phenylene))bis(methylene)dipiperidine-2-carboxylic acid di-trifluoroacetic acid salt,
(2S,2′S)-1,1′-(((((2,2′-Dimethyl-[1,1′-biphenyl]-3,3′-diyl)bis(methylene))bis(oxy))bis(3-(trifluoromethyl)-4,1-phenylene))bis(methylene))bis(piperidine-2-carboxylic acid),
(2S,2'S,4S,4′S)-1,1′-(((((2,2′-Dimethyl-[1,1′-biphenyl]-3,3′-diyl)bis(methylene))bis(oxy))bis(3-bromo-4,1-phenylene))bis(methylene))bis(4-hydroxypyrrolidine-2-carboxylic acid),
(2S,2′S)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))dipropionic acid di-trifluoroacetic acid salt,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(2-methylpropane-1,3-diol) di-trifluoroacetic acid salt,
2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(ethan-1-ol) di-trifluoroacetic acid salt, and
(2R,2′R,3S,3′S)-2,2′-((((((2,2′-Dimethyl-[1,1′-biphenyl]-3,3′-diyl)bis(methylene))bis(oxy))bis(3-bromo-4,1-phenylene))bis(methylene))bis(azanediyl))bis(3-hydroxybutanoic acid) di-trifluoroacetic acid salt.

20. The method of claim 18, wherein the PD-1/PD-L1 dependent condition is colon cancer, gastric cancer, thyroid cancer, lung cancer, leukemia, pancreatic cancer, melanoma, multiple melanoma, brain cancer, CNS cancer, renal cancer, prostate cancer, ovarian cancer or breast cancer.
